(19) United States
(12) Patent Application Publication
(10) Pub. No.: US 2026/0274798 A1
(43) Pub. Date: Sep. 17, 2026

(54) PENTAMIDINE ANALOGUE POLYMORPHS, FORMULATIONS, AND METHODS THEREOF

(71) Applicant: AURANSA INC., Palo Alto, CA (US)

(72) Inventors: Gregory R. LUEDTKE, Salinas, CA (US); Andrew A. PROTTER, Palo Alto, CA (US); Pek Yee LUM, Palo Alto, CA (US); Anup Ramkrishna BARDE, Balewadi (IN); Shreenivas Digamber GUNDALE, Pune (IN); Dattatraya Bhausaheb JADHAV, Ahmednagar (IN)

(73) Assignee: AURANSA INC., Palo Alto, CA (US)

(21) Appl. No.: 19/473,456

(22) PCT Filed: Apr. 7, 2024
(86) PCT No.: PCT/US2024/023513

§ 371 Date: Oct. 7, 2025

Related U.S. Application Data
(60) Provisional application No. 63/494,880, filed on Apr. 7, 2023.

(51) Int. Cl. C07D 213/81; A61K 9/48; A61K 31/455

(52) U.S. Cl. CPC C07D 213/81; A61K 9/485; A61K 9/4858; A61K 9/4866; A61K 31/455

(57) ABSTRACT

The present invention relates to crystalline forms of 5-((5-(4-carbamimidoylphenoxy)pentyl)oxy)picolinimidamide (Compound A) or salts or solvates thereof. Also provided are methods of preparing Compound (A) and a pharmaceutical salt thereof, as well as pharmaceutical compositions comprising the crystalline forms, and their methods and/or uses for treatment of cancer.

### FIELD OF THE INVENTION

The present invention relates to a pentamidine analogue or salt, solvates or forms thereof, corresponding pharmaceutical compositions, methods of making and of treating cancer.

### BACKGROUND

Crystalline forms have advantageous properties such as ease of isolation, enhanced purity, and greater physical and chemical stability compared to analogous amorphous forms. These attributes can be particularly important for pharmaceutical agents where large-scale production, reproducibility, and compound purity are required. Crystalline forms of compounds may be uniquely advantageous as the corresponding amorphous forms are often unsuitable for formulating, such as encapsulation or tableting.

An analogue of pentamidine was described as Compound 1 (hereinafter referred to as “Compound A”) in PCT publication WO 2020/132636 A1, the disclosure of which is incorporated herein by reference in its entirety.

There remains a need in the art to develop effective pharmaceutical vehicles, such as pharmaceutical compositions, to deliver therapeutic agents to treat and prevent cancer.

The present invention addresses these needs by providing pharmaceutical compositions, or pharmaceutically acceptable salt or solvate forms thereof of Compound A.

### BRIEF SUMMARY

Provided herein are crystalline forms of 5-((5-(4-carbamimidoylphenoxy)pentyl)oxy)picolinimidamide (Compound A) or salts or solvates thereof, as well as methods of preparing Compound A, pharmaceutical compositions, methods and/or uses for treatment of cancer.

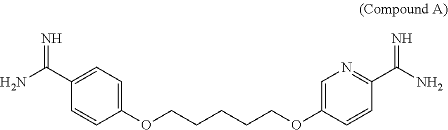 [SMILES: N=C(N)c1ccc(OCCCCCOc2ccc(C(=N)N)nc2)cc1]

In general, the present invention relates to Compound A or salts thereof, or solvates or forms of any of the foregoing, corresponding pharmaceutical compositions, methods and/or uses for treatment of cancer.

In one aspect, provided herein are methods of preparing Compound A, or a salt thereof, or a solvate of the foregoing.

In another aspect, the present invention also provides a crystalline form of Compound A, or a pharmaceutically acceptable salt thereof, or a solvate of the foregoing. The pharmaceutically acceptable salts of Compound A provided herein include acetate salt and phosphate salt.

The present invention further provides pharmaceutical compositions comprising crystalline Form 1 of the diacetate salt of Compound A or solvate thereof, and a pharmaceutically acceptable excipient, characterized by an X-ray powder diffraction (XRPD) pattern substantially as set forth in FIG. 1.

The present invention further provides pharmaceutical compositions comprising crystalline Form 2 of the diacetate salt of Compound A or solvate thereof, and a pharmaceutically acceptable excipient, characterized by an X-ray powder diffraction (XRPD) pattern substantially as set forth in FIG. 14.

The present invention further provides pharmaceutical compositions comprising crystalline Form 3 of the diacetate salt of Compound A or solvate thereof, and a pharmaceutically acceptable excipient, characterized by an X-ray powder diffraction (XRPD) pattern substantially as set forth in FIG. 15.

The present invention further provides pharmaceutical compositions comprising crystalline Form 4 of the diacetate salt of Compound A or solvate thereof, and a pharmaceutically acceptable excipient, characterized by an X-ray powder diffraction (XRPD) pattern substantially as set forth in FIG. 26.

The present invention further provides pharmaceutical compositions comprising crystalline Form 5 of the diacetate salt of Compound A or solvate thereof, and a pharmaceutically acceptable excipient, characterized by an X-ray powder diffraction (XRPD) pattern substantially as set forth in FIG. 27.

The present invention further provides pharmaceutical compositions comprising crystalline Form 6 of the diacetate salt of Compound A or solvate thereof, and a pharmaceutically acceptable excipient, characterized by an X-ray powder diffraction (XRPD) pattern substantially as set forth in FIG. 32.

The present invention further provides pharmaceutical compositions comprising crystalline Form 7 of the diacetate salt of Compound A or solvate thereof, and a pharmaceutically acceptable excipient, characterized by an X-ray powder diffraction (XRPD) pattern substantially as set forth in FIG. 37.

The present invention further provides pharmaceutical compositions comprising crystalline Form 8 of the diacetate salt of Compound A or solvate thereof, and a pharmaceutically acceptable excipient, characterized by an X-ray powder diffraction (XRPD) pattern substantially as set forth in FIG. 42.

The present invention further provides pharmaceutical compositions comprising crystalline Form 9 of the diacetate salt of Compound A or solvate thereof, and a pharmaceutically acceptable excipient, characterized by an X-ray powder diffraction (XRPD) pattern substantially as set forth in FIG. 53.

The present invention further provides pharmaceutical compositions comprising crystalline Form 11 of the diacetate salt of Compound A or solvate thereof, and a pharmaceutically acceptable excipient, characterized by an X-ray powder diffraction (XRPD) pattern substantially as set forth in FIG. 64.

The present invention further provides pharmaceutical compositions comprising crystalline Form 12 of the phosphate salt of Compound A or solvate thereof, and a pharmaceutically acceptable excipient, characterized by an X-ray powder diffraction (XRPD) pattern substantially as set forth in FIG. 65.

The present invention further provides pharmaceutical compositions comprising crystalline Form 13 of the diacetate salt of Compound A or solvate thereof, and a pharmaceutically acceptable excipient, characterized by an X-ray powder diffraction (XRPD) pattern substantially as set forth in FIG. 66.

Also provided herein is a composition comprising an acetate salt form of Compound A, or a solvate thereof, in an amount of from about 0.1% to about 60% (w/w) of the composition; and one or more pharmaceutically acceptable excipients.

Further provided herein is a composition comprising a crystalline form of Compound A, or a pharmaceutically acceptable salt thereof, or a solvate of the foregoing, in an amount of from about 0.1% to about 60% (w/w) of the composition; and one or more pharmaceutically acceptable excipients.

Provided herein is a method including forming a mixture comprising a silicified microcrystalline cellulose, lactose monohydrate, and colloidal silicon dioxide; adding an acetate salt form of Compound A, or a solvate thereof and blending the mixture; adding magnesium stearate and blending the mixture; and filling capsule shells with the blend.

Further provided are capsules formed by the methods described herein.

The present invention provides a method of treating cancer, which comprises administering to the subject a therapeutically effective amount of a composition of the present invention to a subject or patient in need thereof as described herein.

The present invention provides use of the compositions of the present invention in the manufacture of a medicament for treating cancer.

### BRIEF DESCRIPTION OF THE DRAWINGS

FIG. 1 shows an X-ray powder diffraction (XRPD) pattern of crystalline Form 1 of the diacetate salt of Compound A.

FIG. 2 shows a thermogravimetric analysis/differential scanning calorimetry (TG/DSC) thermogram of crystalline Form 1 of the diacetate salt of Compound A.

FIG. 3 shows a DSC first heat thermogram of crystalline Form 1 of the diacetate salt of Compound A.

FIG. 4 shows a DSC cool thermogram of crystalline Form 1 of the diacetate salt of Compound A.

FIG. 5 shows a DSC second heat thermogram of crystalline Form 1 of the diacetate salt of Compound A.

FIG. 6 shows a DSC straight heat thermogram from 20° C. to 275° C. of crystalline Form 1 of the diacetate salt of Compound A.

FIG. 7 shows a dynamic vapor sorption (DVS) isotherm curve of crystalline Form 1 of the diacetate salt of Compound A taken at 25° C.

FIG. 8 shows a DVS kinetic plot of crystalline Form 1 of the diacetate salt of Compound A taken at 25° C.

FIG. 9 shows polarized light microscopy (PLM) images of crystalline Form 1 of The diacetate salt of Compound A.

FIG. 10 shows a Fourier transform infrared (FT-IR) spectrum of crystalline Form 1 of the diacetate salt of Compound A.

FIG. 11 shows a ¹H nuclear magnetic resonance (¹H NMR) spectrum of crystalline Form 1 of the diacetate salt of Compound A.

FIG. 12 shows a diode array detector (DAD) spectrum of crystalline Form 1 of the diacetate salt of Compound A.

FIG. 13 shows a liquid chromatography mass spectrometry (LC-MS) spectrum of crystalline Form 1 of the diacetate salt of Compound A.

FIG. 14 shows an XRPD pattern of crystalline Form 2 of the diacetate salt of Compound A.

FIG. 15 shows an XRPD pattern of crystalline Form 3 of the diacetate salt of Compound A.

FIG. 16 shows a ¹H NMR spectrum of crystalline Form 3 of the diacetate salt of Compound A.

FIG. 17 shows a TG/DSC thermogram of crystalline Form 3 of the diacetate salt of Compound A.

FIG. 18 shows a DSC first heat thermogram of crystalline Form 3 of the diacetate salt of Compound A.

FIG. 19 shows a DSC cool thermogram of crystalline Form 3 of the diacetate salt of Compound A.

FIG. 20 shows a DSC second heat thermogram of crystalline Form 3 of the diacetate salt of Compound A.

FIG. 21 shows PLM images of crystalline Form 3 of the diacetate salt of Compound A.

FIG. 22 shows a FT-IR spectrum of crystalline Form 3 of the diacetate salt of Compound A.

FIG. 23 shows a DVS isotherm curve of crystalline Form 3 of the diacetate salt of Compound A taken at 25° C.

FIG. 24 shows a DVS kinetic plot of crystalline Form 3 of the diacetate salt of Compound A taken at 25° C.

FIG. 25 shows a DSC straight heat thermogram from 20° C. to 150° C. of crystalline Form 3 of the diacetate salt of Compound A.

FIG. 26 shows an XRPD pattern of crystalline Form 4 of the diacetate salt of Compound A.

FIG. 27 shows an XRPD pattern of crystalline Form 5 of the diacetate salt of Compound A.

FIG. 28 shows a ¹H NMR spectrum of crystalline Form 5 of the diacetate salt of Compound A.

FIG. 29 shows a TG/DSC thermogram of crystalline Form 5 of the diacetate salt of Compound A.

FIG. 30 shows PLM images of crystalline Form 5 of the diacetate salt of Compound A.

FIG. 31 shows a FT-IR spectrum of crystalline Form 5 of the diacetate salt of Compound A.

FIG. 32 shows an XRPD pattern of crystalline Form 6 of the diacetate salt of Compound A.

FIG. 33 shows a ¹H NMR spectrum of crystalline Form 6 of the diacetate salt of Compound A.

FIG. 34 shows a TG/DSC thermogram of crystalline Form 6 of the diacetate salt of Compound A.

FIG. 35 shows PLM images of crystalline Form 6 of the diacetate salt of Compound A.

FIG. 36 shows a FT-IR spectrum of crystalline Form 6 of The diacetate salt of Compound A.

FIG. 37 shows an XRPD pattern of crystalline Form 7 of the diacetate salt of Compound A.

FIG. 38 shows a ¹H NMR spectrum of crystalline Form 7 of the diacetate salt of Compound A.

FIG. 39 shows a TG/DSC thermogram of crystalline Form 7 of the diacetate salt of Compound A.

FIG. 40 shows PLM images of crystalline Form 7 of the diacetate salt of Compound A.

FIG. 41 shows a FT-IR spectrum of crystalline Form 7 of the diacetate salt of Compound A.

FIG. 42 shows an XRPD pattern of crystalline Form 8 of the diacetate salt of Compound A.

FIG. 43 shows a ¹H NMR spectrum of crystalline Form 8 of the diacetate salt of Compound A.

FIG. 44 shows a TG/DSC thermogram of crystalline Form 8 of the diacetate salt of Compound A.

FIG. 45 shows a DSC first heat thermogram of crystalline Form 8 of the diacetate salt of Compound A.

FIG. 46 shows a DSC cool thermogram of crystalline Form 8 of the diacetate salt of Compound A.

FIG. 47 shows a DSC second heat thermogram of crystalline Form 8 of the diacetate salt of Compound A.

FIG. 48 shows PLM images of crystalline Form 8 of the diacetate salt of Compound A.

FIG. 49 shows a FT-IR spectrum of crystalline Form 8 of the diacetate salt of Compound A.

FIG. 50 shows a DVS isotherm curve of crystalline Form 8 of the diacetate salt of Compound A taken at 25° C.

FIG. 51 shows a DVS kinetic plot of crystalline Form 8 of the diacetate salt of Compound A taken at 25° C.

FIG. 52 shows a DSC straight heat thermogram from 20° C. to 150° C. of crystalline Form 8 of the diacetate salt of Compound A.

FIG. 53 shows an XRPD pattern of crystalline Form 9 of the diacetate salt of Compound A.

FIG. 54 shows a ¹H NMR spectrum of crystalline Form 9 of the diacetate salt of Compound A.

FIG. 55 shows a TG/DSC thermogram of crystalline Form 9 of the diacetate salt of Compound A.

FIG. 56 shows a DSC first heat thermogram of crystalline Form 9 of the diacetate salt of Compound A.

FIG. 57 shows a DSC cool thermogram of crystalline Form 9 of the diacetate salt of Compound A.

FIG. 58 shows a DSC second heat thermogram of crystalline Form 9 of the diacetate salt of Compound A.

FIG. 59 shows PLM images of crystalline Form 9 of the diacetate salt of Compound A.

FIG. 60 shows a FT-IR spectrum of crystalline Form 9 of the diacetate salt of Compound A.

FIG. 61 shows a DVS isotherm curve of crystalline Form 9 of the diacetate salt of Compound A taken at 25° C.

FIG. 62 shows a DVS kinetic plot of crystalline Form 9 of the diacetate salt of Compound A taken at 25° C.

FIG. 63 shows a DSC straight heat thermogram from 20° C. to 100° C. of crystalline Form 9 of the diacetate salt of Compound A.

FIG. 64 shows an XRPD pattern of crystalline Form 11 of The diacetate salt of Compound A.

FIG. 65 shows an XRPD pattern of crystalline Form 12 of the phosphate salt of Compound A.

FIG. 66 shows an XRPD pattern of crystalline Form 13 of the diacetate salt of Compound A.

FIG. 67 shows a phase diagram for several forms of salts of Compound A.

### DETAILED DESCRIPTION

### I. General

The present invention relates to 5-((5-(4-carbamimidoylphenoxy)pentyl)oxy)picolinimidamide (Compound A), salts or solvates thereof, or crystalline forms of any of the foregoing, methods of preparing Compound A, or salts or solvates thereof, or crystalline forms of any of the foregoing, and pharmaceutical compositions comprising Compound A, or salts or solvates thereof, or crystalline forms of any of the foregoing.

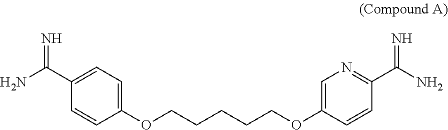 [SMILES: N=C(N)c1ccc(OCCCCCOc2ccc(C(=N)N)nc2)cc1]

### II. Definitions

“A,” “an,” or “a(n)”, is an indefinite article when used in reference to a group of substituents or “substituent group” herein, mean at least one.

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3. In some embodiments, reference to about a value or parameter includes a description of that value or parameter per se. For example, reference to about 20 molar equivalents includes and describes 20 molar equivalents per se.

“Administering” refers to administration of the composition of the present invention to a subject.

“Composition” or “Pharmaceutical Composition” as used herein is intended to encompass an invention or product comprising the specified active product ingredient (API), which may include pharmaceutically acceptable excipients, carriers or diluents as described herein, such as in specified amounts defined throughout the originally filed disclosure, which results from combination of specific components, such as specified ingredients in the specified amounts as described herein.

“Granulated mixture” refers to a mixture of two or more agents made by mixing the two or more agents and granulating them together in a particulate form. Such a mixture provides particulate material that is composed of two or more agents. For example, in the present invention, the compositions may include, but are not limited to granulated mixtures of an acetate salt form of the Compound A or solvate thereof and absorption or permeation enhancer, such as sodium caprate. Such a granulated mixture is formed into a particle or tablet forms, which contain an acetate salt form of Compound A or solvate thereof and sodium caprate. In some embodiments, the compositions may include granulated mixtures comprising sodium caprate.

“Disintegrant” refers to a pharmaceutical excipient that is incorporated into a composition to promote their disintegration when they come into contact with a liquid. For example, a disintegrant is a pharmaceutically acceptable agent, used in preparation of tablets, which causes tablets to disintegrate and release medicinal substances on contact with moisture. Examples of disintegrants include, without limitation, crosslinked polymers, including crosslinked polyvinylpyrrolidone (crospovidone), crosslinked sodium carboxymethyl cellulose (croscarmellose sodium), and modified starch sodium starch glycolate and the like. Representative disintegrants for use in the present invention, may include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums (like gellan), low-substituted hydroxypropyl cellulose, or mixtures thereof and the like. In some aspects, disintegrants for use in the present invention, may include, but are not limited to croscarmellose sodium. Additional representative disintegrants for use in the present invention, may include, but are not limited to microcrystalline cellulose, croscarmellose sodium, alginic acid, sodium alginate, crospovidone, cellulose, agar and related gums, sodium starch glycolate, corn starch, potato starch, sodium starch glycolate. Veegum HV, methylcellulose, agar, bentonite, carboxymethylcellulose, alginic acid, guar gum combinations thereof, and the like. Representative disintegrants for use in the present invention, include, but are not limited to, starches, clays, celluloses, alginates and gums and crosslinked starches, celluloses and polymers, combinations thereof and the like.

“Disposed over” refers to the placement of one phase or coating on top of another phase or coating. Such placement can conform to the shape of the underlying phase or coating such that the layering of phases and coatings do not leave substantial gaps there between.

“Enteric coating” refers to any of the commonly applied polymeric coatings employed for delayed release of active ingredients. As conventionally understood in the art, an enteric coating generally is a polymer barrier applied to oral medication that prevents its dissolution or disintegration in the gastric environment. This helps by either protecting drugs from the acidity of the stomach, the stomach from the detrimental effects of the drug, or to release the drug after the stomach (usually in the upper tract of the intestine). Some drugs are unstable at the pH of gastric acid and need to be protected from degradation. An enteric coating is also an effective method to obtain drug targeting (such as gastro-resistant drugs). Such delayed release is typically pH dependent and allows for release of the active ingredient further in the intestinal tract where the pH differs from that in the stomach. In general, suitable materials used for enteric coatings may include, but is not limited to fatty acids, waxes, shellac, plastics, and plant fibers, where such enteric materials, may include, but is not limited to cellulose acetate phthalate, polyvinylalcohol phthalate, shellac, zein, hydroxypropylmethyl cellulose phthalate, cellulose acetate trimaleate, film resins, etc and the like. Additional examples of enteric coating for use in the present invention, may include, without limitation, those based on esters of aleurtic acid, cellulose acetate phthalate (CAP), poly(methacrylic acid-co-methyl methacrylate), poly(vinyl acetate phthalate) (PVAP), cellulose acetate trimellitate (CAT), hydroxypropyl methylcellulose phthalate (HPMCP) and the like. Other suitable materials used for enteric coatings may also include, but is not limited to poly(methacrylic acid ethyl acrylate) (L100D-55), combinations of methyl acrylate, methyl methacrylate, hydroxypropyl methylcellulose (HPMC), methacrylic acid (FS30D), hydroxypropyl methylcellulose acetate succinate (HPMC-AS), and Type L HPMC-AS. In some embodiments, the enteric coating is disposed over the subcoating.

“Glidant” refers to a substance that is added to a powder to improve its flowability and/or lubricity. Examples of glidants, may include, but is not limited to, magnesium stearate, fumed silica, starch, talc and the like.

“Silica” refers to a pharmaceutical excipient that can be employed as flow agent (anti-caking), adsorbent and desiccant in solid product forms. It can also be used to increase the mechanical stability and the disintegration rate of the compositions. The silica can be fumed, i.e., referring to its production through a pyrogenic process to generate fine particles of silica. Particles of fumed silica can vary in size such as from 5 nm to 100 nm, or from 5 to 50 nm. The particles can be non-porous and have a surface area from 50-1,000 m²/g or from 50-600 m²/g. Examples of silicas include Aerosil 200, having a specific surface area of about 200 m²/g. The silica can be hydrophilic. Examples of suitable silica materials include, but are not limited to SiO₂, colloidal silicon dioxide, aerosol, colloidal silica, fumed silica, silicon dioxide fumed, colloidal anhydrous silica, colloidal silicon dioxide, and the like.

“Lubricant” refers to a substance added to a formulation to reduce friction. Compounds that serve as lubricants can also have properties as glidants. Examples of lubricants may include, but are not limited to, talc, silica, and fats such as vegetable stearin, magnesium stearate or stearic acid and the like.

“Microcrystalline cellulose,” or “MCC,” refers to a pharmaceutical grade of cellulose manufactured from a refined wood pulp. The MCC can be unmodified or chemically modified, such as silicified microcrystalline cellulose (SMCC). MCC can serve the function of a bulking agent and aid in tablet formation due to its favorable compressibility characteristics.

“Patient” or “subject” refers to a living organism, which includes, but is not limited to a human subject suffering from or prone to a disease or condition that can be treated by administration of a pharmaceutical composition as provided herein. Further non-limiting examples may include, but is not limited to humans, other mammals, bovines, rats, mice, dogs, monkeys, goat, sheep, cows, deer, horse, and other mammalian animals and the like. In some aspects, the patient is human.

By “pharmaceutically acceptable” it is meant the carrier(s), diluent(s) or excipient(s) must be compatible with the other components or ingredients of the compositions of the present invention, i.e., that which is useful, safe, non-toxic acceptable for pharmaceutical use. In accordance with the present invention pharmaceutically acceptable means approved or approvable as is listed in the U.S. Pharmacopoeia or other generally recognized pharmacopoeia for use in animals, and more particularly, in humans.

“Free base” refers to 5-((5-(4-carbamimidoylphenoxy)pentyl)oxy)picolinimidamide (Compound A) with the following structure:

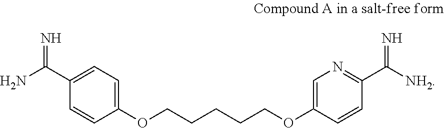 [SMILES: N=C(N)c1ccc(OCCCCCOc2ccc(C(=N)N)nc2)cc1]

“Crystalline salt” refers to a crystalline form of a pharmaceutically acceptable salt of Compound A, which may include, but is not limited to a crystalline acetate salt of Compound A and a crystalline phosphate salt of Compound A.

Compositions or pharmaceutical compositions of the present invention may be in different pharmaceutically acceptable forms, which may include, but are not limited to a liquid composition, a tablet or matrix composition, a capsule composition, etc. and the like. When the composition is a tablet composition, the tablet may include, but is not limited to different layers. The tablet composition can also include, but is not limited to one or more coatings.

“Silicified microcrystalline cellulose,” or “SMCC,” refers to a particulate agglomerate of coprocessed microcrystalline cellulose and silicon dioxide. Suitable for use in the present invention, SMCC may include, but is not limited to amounts from about 0.1% to about 20% silicon dioxide, by weight of the microcrystalline cellulose, where the silicon dioxide can have a particle size from about 1 nanometer (nm) to about 100 microns (μm), based on average primary particle size. For example, the silicon dioxide can contain from about 0.5% to about 10% of the silicified microcrystalline cellulose, or from about 1.25% to about 5% by weight relative to the microcrystalline cellulose. Moreover, the silicon dioxide can have a particle size from about 5 nm to about 40 m, or from about 5 nm to about 50 m. The silicon dioxide can have a surface area from about 10 m²/g to about 500 m²/g, or from about 50 m²/g to about 500 m²/g, or from about 175 m²/g to about 350 m²/g. Silicified microcrystalline cellulose is commercially available from a number of suppliers known to one of skill in the art, Including Penwest Pharmaceuticals, Inc., under the trademark PROSOLV®. PROSOLV® is available in a number of grades, including, for example, PROSOLV® SMCC 50, PROSOLV® SMCC 90, and PROSOLV® HD. Other products include, without limitation, SMCC 50LD, SMCC HD90 and SMCC 90LM and the like.

“Solvate” as used herein, means a physical association of Compound A. or a salt thereof, of the present invention with one or more solvent molecules. This physical association involves varying degrees bonding, including hydrogen bonding. In certain instances, the solvate will be capable of isolation. The term “solvate” is intended to encompass both solution-phase and isolatable solvates. Non-limiting examples of suitable solvates include hydrates.

“Sorbitol” refers to the sugar alcohol D-glucitol and which may serve as a binder promoting adhesion of ingredients in tablet compositions.

“Sugar alcohol” as used herein refers to compounds derived from sugars and containing one or more hydroxyl groups. Sugar alcohol may contain multiple —OH groups and be classified as polyols. Examples of sugar alcohol include but not limited to sorbitol, mannitol, xylitol.

“Subcoating” refers to any number of film layers disposed over the core tablet that can provide one or more benefits such as, providing a smooth tablet surface to ease swallowing of compositions, accommodate pigmentation to aid in pill identification, provide a moisture barrier, and provide a high tensile strength outer layer of the tablet. Such subcoatings can comprise, but is not limited to graft co-polymers of polyvinyl alcohol (PVA) and polyethylene glycol (PEG). Commercial products that provide subcoatings include the line of products under the trade names OPADRY®, OPAGLOS®, and the like. A subcoating may be further covered by one or more additional coatings.

In some embodiments, the subcoating refers to any number of film layers disposed over the core tablet. Examples of suitable materials for cosmetic subcoatings include a polyvinyl alcohol-polyethylene glycol (PVA-PEG) graft co-polymer (e.g., OPADRY® QX). Other coatings include, without limitation, HPMC, HPC, PVA, Eudragit E based coatings and the like.

In some embodiments, a subcoating may be further covered by one or more additional coatings, such as an enteric coating or a functional coating. In certain embodiments, a subcoating comprises one or more of a plasticizer, anti-tacking agent, coloring agent, HPMC, HPC, PVA, and Eudragit E based coatings. In some embodiments, a subcoating is covered with one or more additional coatings. In certain embodiments, the one or more additional coatings over the subcoating is an enteric coating. In other embodiments, the one or more additional coatings over the subcoating is a functional coating.

In some aspects, a subcoating is not covered by one or more additional coatings and is referred to as a cosmetic subcoating. For example, in certain embodiments, a core tablet is covered by a cosmetic coating and the cosmetic coating is not further covered with an enteric coating or a functional coating. In some embodiments, a cosmetic coating can serve as a smooth surface to aid in swallowing the tablet. In some embodiments, a cosmetic coating can provide a vehicle for pigmentation for tablet identification, serve as a smooth surface to aid in swallowing the tablet.

“Core tablet” refers to a mixture of the components of the core tablet. In some embodiments, the components are one or more of a crystalline form of Compound A, a pharmaceutically acceptable salt thereof, or a solvate of the foregoing, and suitable excipients. In some embodiments, the suitable excipient is one or more of the following, but not limited to, a filler, a disintegrant, a glidant, a lubricant, and an absorption enhancer. A subcoating, a cosmetic coating, an enteric coating, or any combination thereof may be disposed over the core tablet.

“Therapeutically effective amount” refers to an amount of a compound (i.e., Compound A) or of a pharmaceutical composition useful for treating or ameliorating an identified disease or condition, or for exhibiting a detectable therapeutic or inhibitory effect. “Therapeutically effective amount” further includes within its meaning a non-toxic but sufficient amount of the particular drug to which it is referring to provide the desired therapeutic effect. The exact amount required will vary from subject to subject depending on factors such as the patient's general health, the patient's age, etc. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy. 20th Edition. 2003, Gennaro, Ed., Lippincott. Williams & Wilkins).

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating: improving a patient's physical or mental well-being. The treatment or amelioration of symptoms can be based on objective or subjective parameters; including the results of a physical examination, neuropsychiatric exams, and/or a psychiatric evaluation.

Abbreviation, “(V/V)” refers to the phrase “volume for volume”, i.e., the proportion of a particular substance within a mixture, as measured by volume or a volume amount of a component of the composition disclosed herein relative to the total volume amount of the composition. Accordingly, the quantity is unit less and represents a volume percentage amount of a component relative to the total volume of the composition. For example, a 2% (V/V) solvent mixture can indicate 2 mL of one solvent is present in 100 mL of the solvent mixture.

Abbreviation, “(w/w)” refers to the phrase “weight for weight”, i.e., the proportion of a particular substance within a mixture, as measured by weight or mass or a weight amount of a component of the composition disclosed herein relative to the total weight amount of the composition. Accordingly, the quantity is unit less and represents a weight percentage amount of a component relative to the total weight of the composition. For example, a 2% (w/w) solution can indicate 2 grams of solute is dissolved in 100 grams of solution.

Systemic routes of administration as conventionally understood in the medicinal or pharmaceutical arts, refer to or are defined as a route of administration of drug, a pharmaceutical composition or formulation, or other substance into the circulatory system so that various body tissues and organs are exposed to the drug, formulation or other substance. As conventionally understood in the art, administration can take place orally (where drug or oral preparations are taken by mouth, and absorbed via the gastrointestinal tract), via enteral administration (absorption of the drug also occurs through the gastrointestinal tract) or parenteral administration (generally injection, infusion, or implantation, etc.

Bioavailability refers to the extent and rate at which the active moiety (drug or metabolite) enters systemic circulation, thereby accessing the site of action. Bioavailability of a drug is impacted by the properties of the dosage form, which depend partly on its design and manufacture.

### III. Crystalline Forms

Provided herein are crystalline forms of carbamimidoylphenoxy)pentyl)oxy)picolinimidamide (Compound A) or salts thereof, or solvates of the foregoing. Crystalline forms of salts of Compound A and solvates of salts of Compound A were unexpectedly obtained and isolated. Crystalline forms of pharmaceutically acceptable salts of Compound A were prepared, isolated, and found suitable for use in pharmaceutical formulations. As such, crystalline forms of compounds may be uniquely advantageous as the corresponding amorphous forms are often unsuitable for formulating, such as in capsules.

In one aspect, the present invention relates to a pharmaceutical composition of a crystalline salt of Compound A:

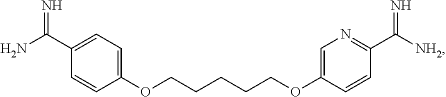 [SMILES: N=C(N)c1ccc(OCCCCCOc2ccc(C(=N)N)nc2)cc1]

or
a pharmaceutically acceptable salt thereof, or a solvate of the foregoing.

In one aspect, the present invention relates to a pharmaceutical composition of an acetate salt of Compound A. In some variations, the acetate salt of Compound A is a diacetate salt.

In another aspect, the present invention relates to a pharmaceutical composition of a phosphate salt of Compound A.

In another aspect, the present invention relates to a pharmaceutical composition of a solvate of an acetate salt of Compound A. In some variations, the solvate is a solvate of a diacetate salt of Compound A. In some variations, the solvate is a hydrate. In some variations the solvate is a monohydrate. In some variations the solvate is a tetrahydrate. In some variations, the solvate is a tetrahydrofuran (THF) solvate. In some variations the solvate is a N,N-dimethylacetamide (DMA) solvate. In some variations the solvate is a N-methylpyrrolidone (NMP) solvate. In some variations the solvate is a ethanol solvate.

In another aspect, the present invention relates to a pharmaceutical composition of a crystalline form of the acetate salt of Compound A. In some variations, the crystalline form is a crystalline form of the diacetate salt of Compound A.

In another aspect, the present invention relates to a pharmaceutical composition of a crystalline form of the phosphate salt of Compound A.

In another aspect, the present invention relates to a pharmaceutical composition of a crystalline form of the solvate of an acetate salt of Compound A. In some variations, the solvate is a solvate of a diacetate salt of Compound A. In some variations, the solvate is a hydrate. In some variations the solvate is a monohydrate. In some variations the solvate is a tetrahydrate. In some variations, the solvate is a tetrahydrofuran (THF) solvate. In some variations the solvate is a N,N-dimethylacetamide (DMA) solvate. In some variations the solvate is a N-methylpyrrolidone (NMP) solvate. In some variations the solvate is a ethanol solvate.

In another aspect, the present invention relates to a pharmaceutical composition of a crystalline form of a pharmaceutically acceptable salt of Compound A. The crystalline form of a pharmaceutically acceptable salt of Compound A may be a crystalline acetate salt or a crystalline phosphate salt of Compound A.

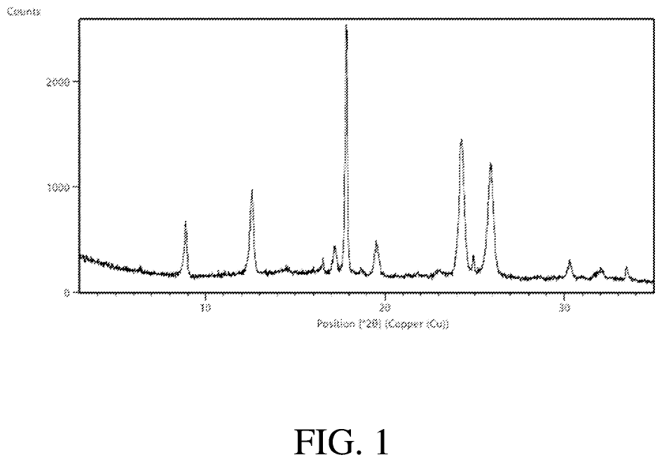

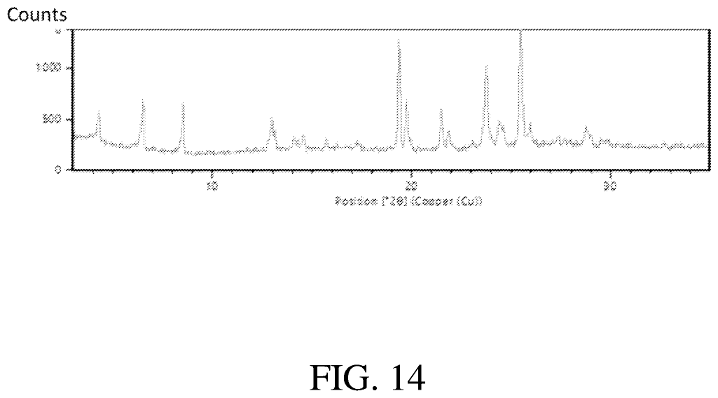

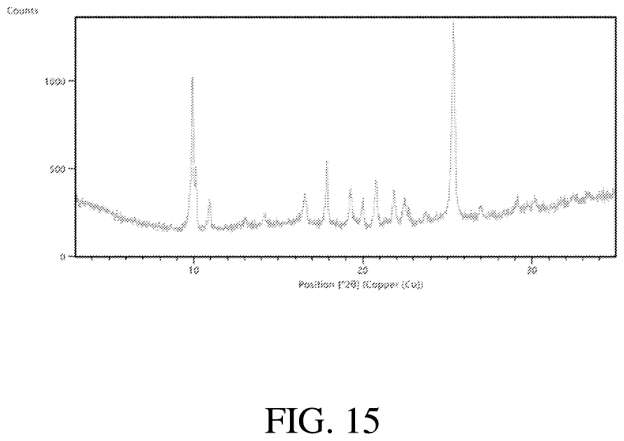

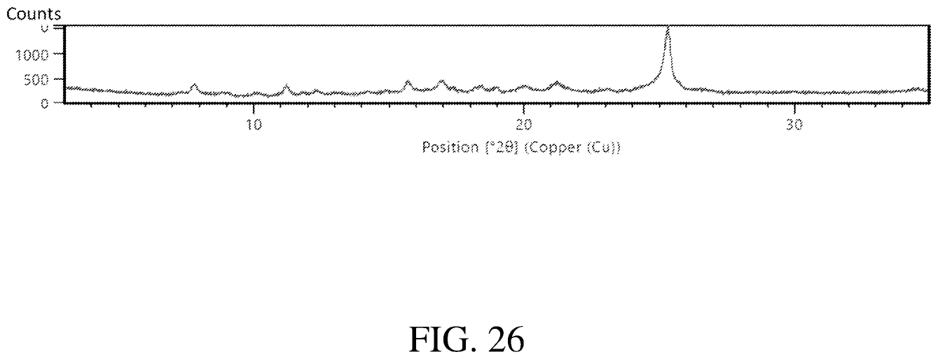

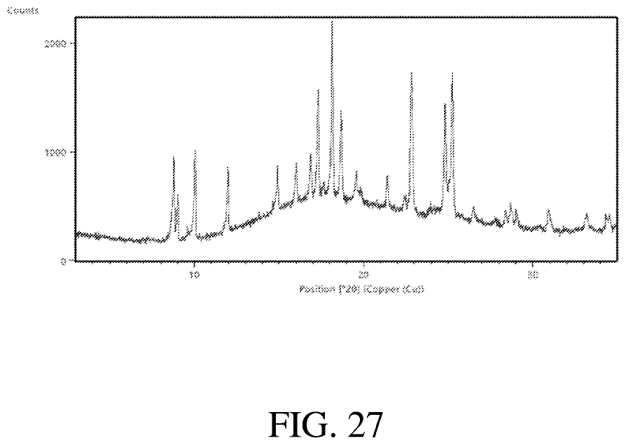

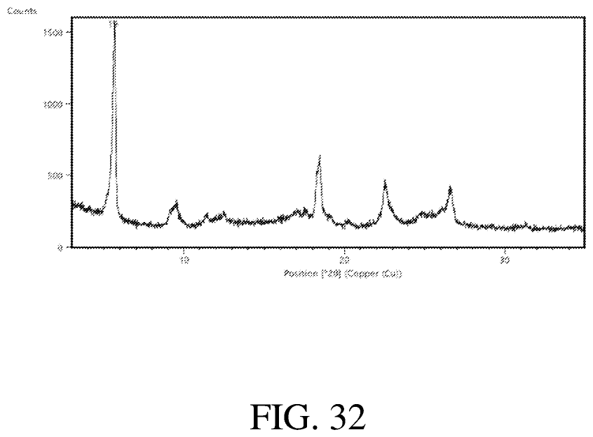

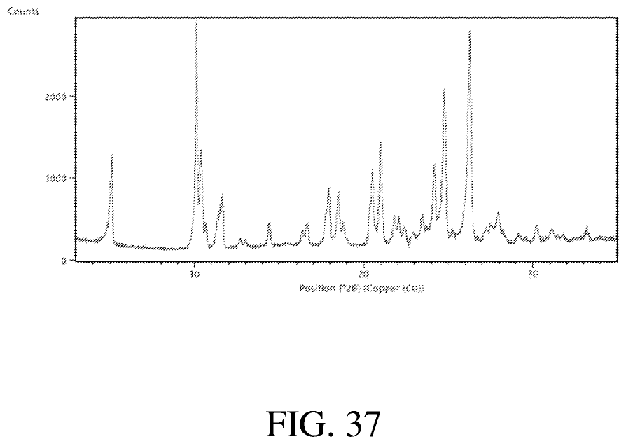

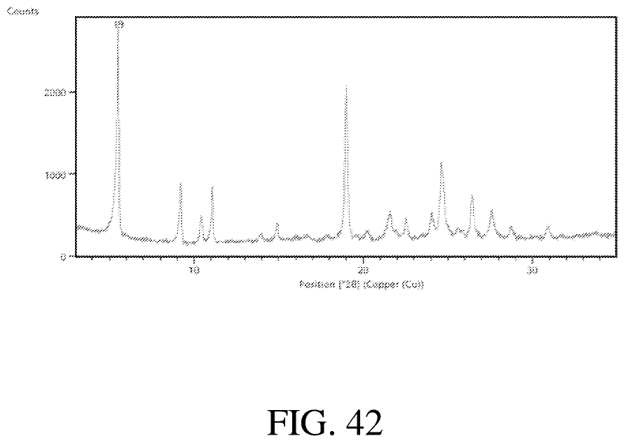

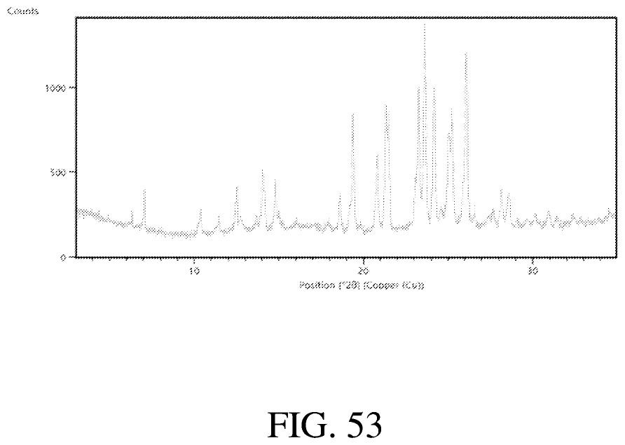

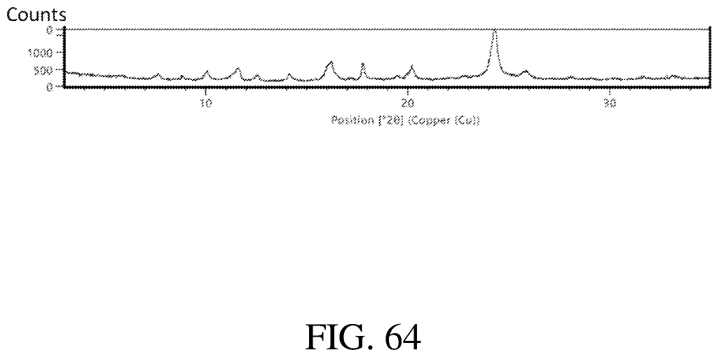

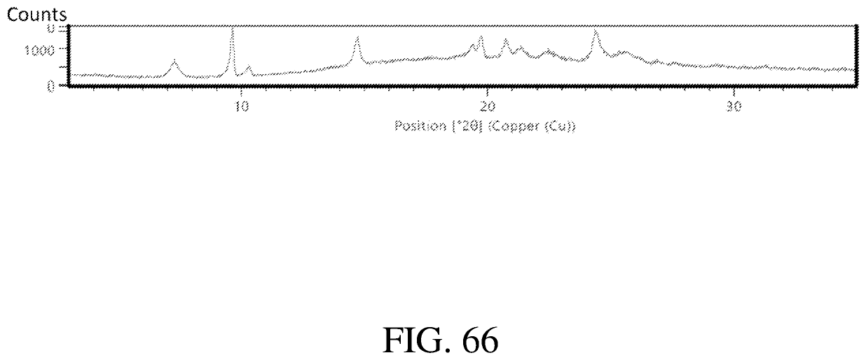

In other aspects, the crystalline acetate salt form of Compound A is characterized by an X-ray powder diffraction (XRPD) pattern substantially as set forth in FIG. 1. In some aspects, the crystalline acetate salt form or solvate thereof is a diacetate salt. In some embodiments, the crystalline acetate salt form of Compound A is characterized by an XRPD pattern substantially as set forth in FIG. 1, FIG. 14, FIG. 15, FIG. 26, FIG. 27, FIG. 32, FIG. 37, FIG. 42, FIG. 53, FIG. 64, or FIG. 66.

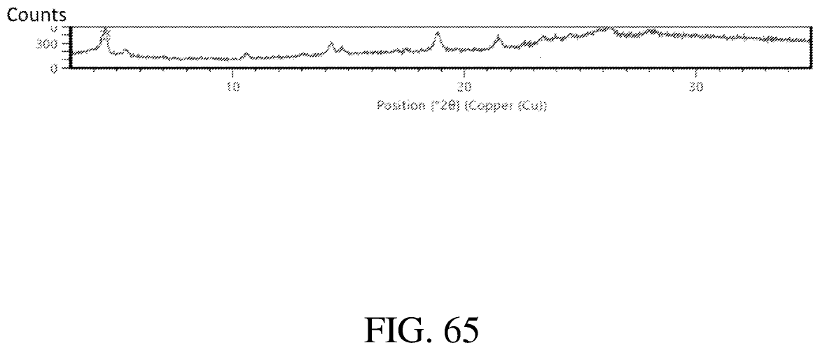

In other aspects, the crystalline phosphate salt form of Compound A is characterized by an X-ray powder diffraction (XRPD) pattern substantially as set forth in FIG. 65.

In some aspects, the present invention relates to a pharmaceutical composition which comprises a therapeutically effective amount of a crystalline salt or solvate thereof described herein and a pharmaceutically acceptable excipient. In some variations, the present invention relates to a pharmaceutical composition which comprises a therapeutically effective amount of a crystalline acetate salt or solvate thereof described herein and a pharmaceutically acceptable excipient. In some variations, the present invention relates to a pharmaceutical composition which comprises a therapeutically effective amount of a crystalline phosphate salt or solvate thereof described herein and a pharmaceutically acceptable excipient.

In some aspects, the present invention relates to a pharmaceutical composition which comprises a therapeutically effective amount of a mixture of two or more crystalline forms of Compound A or salts thereof, or solvates of the foregoing, as described herein, and a pharmaceutically acceptable excipient.

### Salt Forms

In some embodiments, a pharmaceutically acceptable salt of Compound A is an acetate salt. In some embodiments, the acetate salt of Compound A is crystalline. In some embodiments, the acetate salt of Compound A is in the form of a solvate. In certain embodiments, the solvate of the acetate salt of Compound A is a hydrate. In some other embodiments, the acetate salt of Compound A is crystalline and in the form of a solvate.

In other embodiments, a pharmaceutically acceptable salt of Compound A is a phosphate salt. In some embodiments, the phosphate salt of Compound A is crystalline. In some embodiments, the phosphate salt of Compound A is in the form of a solvate. In certain embodiments, the solvate of the phosphate salt of Compound A is a hydrate. In some other embodiments, the phosphate salt of Compound A is crystalline and in the form of a solvate.

### Salt Ratios

In some embodiments, Compound A is in the form of a pharmaceutically acceptable salt. In certain embodiments, the pharmaceutically acceptable salt of Compound A is crystalline. In some embodiments, a crystalline pharmaceutically acceptable salt of Compound A comprises a cationic form of Compound A and a pharmaceutically acceptable anion. For example, a crystalline diacetate salt of Compound A comprises Compound A in its cationic form and two acetate anions. The salt compositions described herein include a salt of Compound A wherein the salt is a pharmaceutically acceptable salt chosen from an acetate salt and a phosphate salt.

In some embodiments, the pharmaceutically acceptable salt of Compound A is an acetate salt and the anion is acetate. In some embodiments, the acetate salt is a diacetate salt. In some embodiments, the pharmaceutically acceptable salt of Compound A is a phosphate salt and the anion is phosphate.

In some embodiments, the molar equivalents of an anion of a crystalline salt of Compound A relative to one mole of Compound A is from about 0.2 to about 2.5. In some embodiments, the molar equivalents of an anion of a salt of Compound A relative to one mole of Compound A is about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, or about 2.5, including any amount in between and fractions thereof.

In some embodiments, the molar equivalents of an acetate anion of a crystalline acetate salt of Compound A relative to one mole of Compound A is from about 0.2 to about 2.5. In some embodiments, the molar equivalents of an acetate anion of a crystalline acetate salt of Compound A relative to one mole of Compound A is from about 1.5 to about 2.5.

### Form 1 of Compound A Diacetate Salt

In some embodiments, the crystalline form of Compound A diacetate salt is crystalline Form 1 of Compound A diacetate salt (hereinafter referred to as “Form 1”). In some embodiments, Form 1 is an anhydrous crystalline form of Compound A diacetate salt In some embodiments, Form 1 is characterized as having an XRPD pattern having two or more diffraction peaks at two theta angles selected from 17.86, 24.21, 24.29, and 25.87+/−0.2 degrees two theta. In some embodiments, Form 1 is characterized as having an XRPD pattern having two or more diffraction peaks at two theta angles selected from 17.86, 24.21, 24.29, and 25.87+/−0.3 degrees two theta. In some embodiments, Form 1 is characterized as having an XRPD pattern having two or more diffraction peaks at two theta angles selected from 17.86, 24.21, 24.29, and 25.87+/−0.4 degrees two theta.

In some embodiments, Form 1 is characterized as having an XRPD pattern having two or more diffraction peaks at two theta angles selected from 3.19, 8.92, 12.62, 16.54, 17.20, 17.86, 19.54, 24.21, 24.29, 24.94, 25.87, 30.29, 32.04, and 33.45+/−0.2 degrees two theta. In some embodiments, Form 1 is characterized as having an XRPD pattern having two or more diffraction peaks at two theta angles selected from 3.19, 8.92, 12.62, 16.54, 17.20, 17.86, 19.54, 24.21, 24.29, 24.94, 25.87, 30.29, 32.04, and 33.45+/−0.3 degrees two theta. In some embodiments, Form 1 is characterized as having an XRPD pattern having two or more diffraction peaks at two theta angles selected from 3.19, 8.92, 12.62, 16.54, 17.20, 17.86, 19.54, 24.21, 24.29, 24.94, 25.87, 30.29, 32.04, and 33.45+/−0.4 degrees two theta.

In some embodiments, Form 1 is characterized as having an XRPD pattern having two or more diffraction peaks at two theta angles selected from 3.19, 5.19, 6.35, 7.18, 8.92, 12.62, 14.55, 15.43, 16.54, 17.20, 17.86, 18.66, 19.54, 21.26, 21.82, 23.05, 24.21, 24.29, 24.94, 25.87, 28.62, 30.29, 31.02, 32.04, and 33.45+/−0.2 degrees two theta. In some embodiments, Form 1 is characterized as having an XRPD pattern having two or more diffraction peaks at two theta angles selected from 3.19, 5.19, 6.35, 7.18, 8.92, 12.62, 14.55, 15.43, 16.54, 17.20, 17.86, 18.66, 19.54, 21.26, 21.82, 23.05, 24.21, 24.29, 24.94, 25.87, 28.62, 30.29, 31.02, 32.04, and 33.45+/−0.3 degrees two theta. In some embodiments, Form 1 is characterized as having an XRPD pattern having two or more diffraction peaks at two theta angles selected from 3.19, 5.19, 6.35, 7.18, 8.92, 12.62, 14.55, 15.43, 16.54, 17.20, 17.86, 18.66, 19.54, 21.26, 21.82, 23.05, 24.21, 24.29, 24.94, 25.87, 28.62, 30.29, 31.02, 32.04, and 33.45+/−0.4 degrees two theta.

In some embodiments, Form 1 is characterized by an XRPD pattern substantially as set forth in FIG. 1.

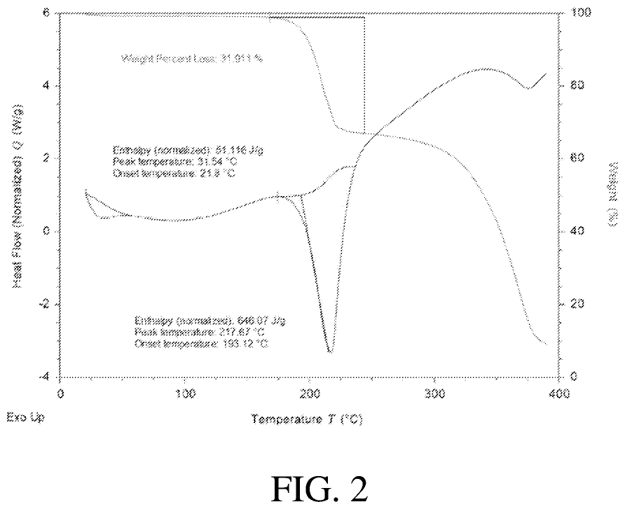

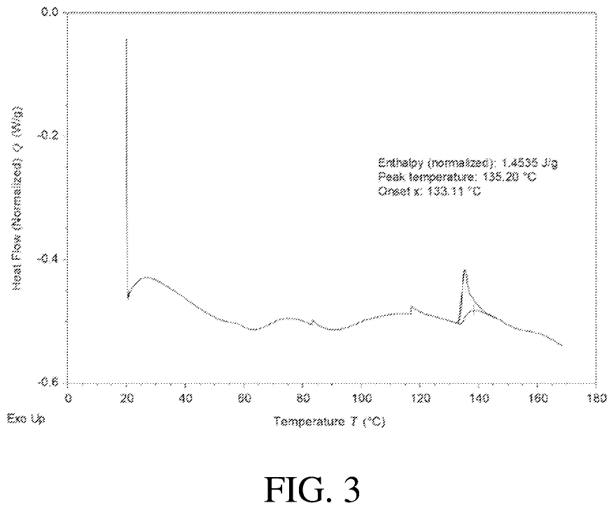

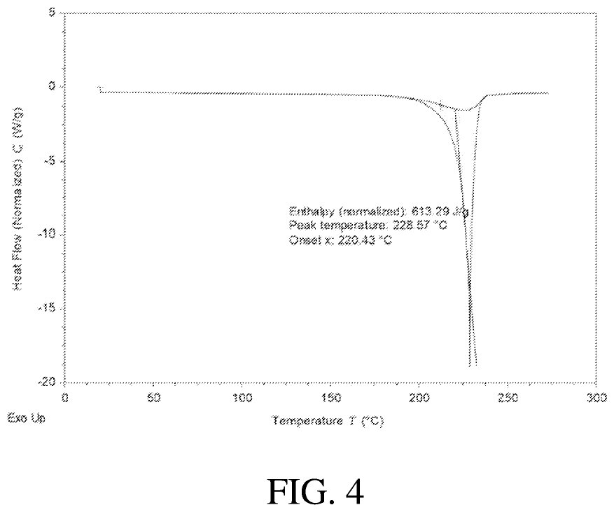

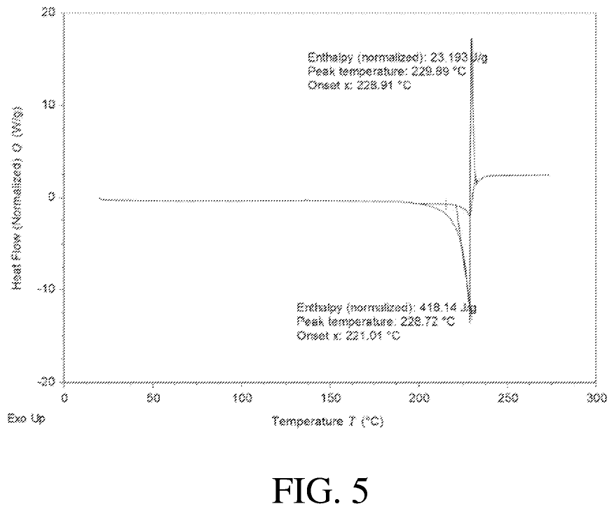

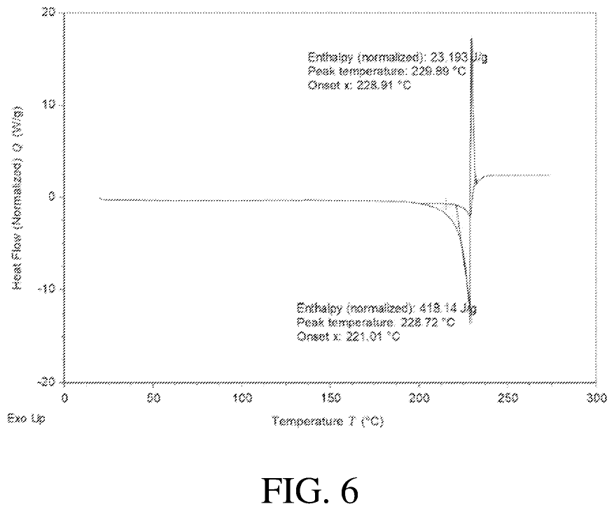

In some embodiments, Form 1 is characterized as having an exothermic peak at about 133.1° C., as determined by differential scanning calorimetry (DSC). In certain embodiments, Form 1 is characterized as having a DSC thermogram substantially as set forth in FIG. 2. In certain embodiments, Form 1 is characterized as having a DSC first heat thermogram substantially as set forth in FIG. 3. In certain embodiments, Form 1 is characterized as having a DSC cool thermogram substantially as set forth in FIG. 4. In certain embodiments, Form 1 is characterized as having a DSC second heat thermogram substantially as set forth in FIG. 5. In certain embodiments, Form 1 is characterized as having a DSC thermogram substantially as set forth in FIG. 6.

In some embodiments, Form 1 is characterized as having a weight loss of about 31.9% at about 193.1° C., as determined by thermogravimetric analysis (TGA). In certain embodiments, Form 1 is characterized as having a TGA graph substantially as set forth in FIG. 2.

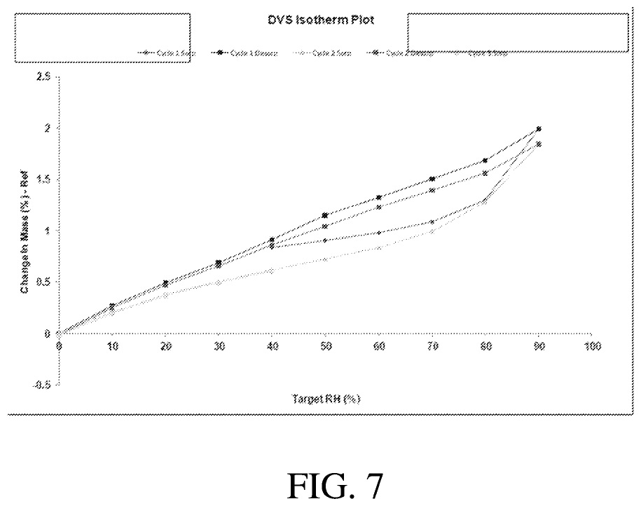

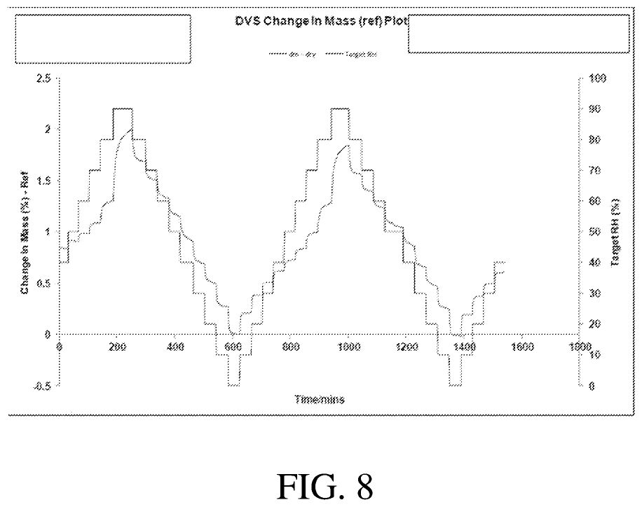

In some embodiments, Form 1 is characterized as having a DVS isotherm curve substantially as shown in FIG. 7. In some embodiments, Form 1 is characterized as having a DVS kinetic plot substantially as shown in FIG. 8.

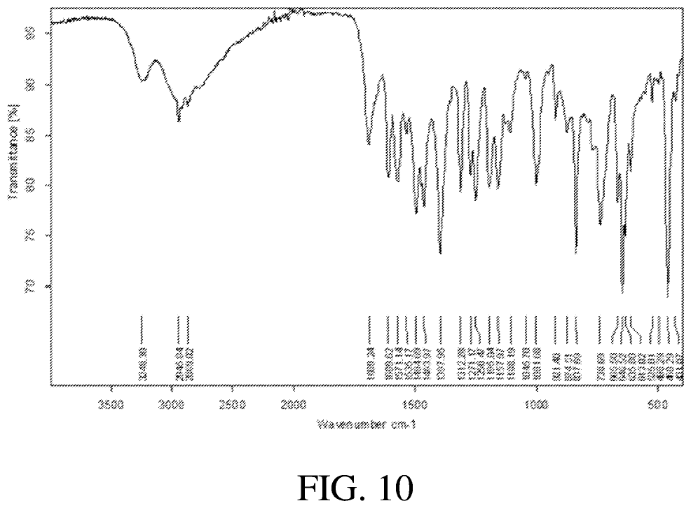

In some embodiments, Form 1 is characterized as having an FT-IR spectrum having peaks selected from 431.07 cm⁻¹, 460.29 cm⁻¹, 498.29 cm⁻¹, 525.01 cm⁻¹, 613.02 cm⁻¹, 635.80 cm⁻¹, 646.52 cm⁻¹, 665.50 cm⁻¹, 738.69 cm⁻¹, 837.69 cm⁻¹. 874.51 cm⁻¹, 921.40 cm⁻¹, 1001.68 cm⁻¹. 1045.70 cm⁻¹, 1108.19 cm⁻¹, 1157.97 cm⁻¹, 1195.04 cm⁻¹, 1250.47 cm⁻¹, 1271.17 cm⁻¹, 1312.28 cm⁻¹, 1397.95 cm⁻¹, 1463.97 cm⁻¹, 1494.69 cm⁻¹, 1535.17 cm⁻¹, 1571.14 cm⁻¹, 1609.62 cm⁻¹, 1689.24 cm⁻¹, 2869.02 cm⁻¹, and 3248.30 cm⁻¹. In some embodiments, Form 1 is characterized as having an FT-IR spectrum having peaks selected from 460.29 cm⁻¹, 1397.95 cm⁻¹ and 1689.24 cm⁻¹. In some embodiments, Form 1 is characterized as having an FT-IR spectrum substantially as shown in FIG. 10.

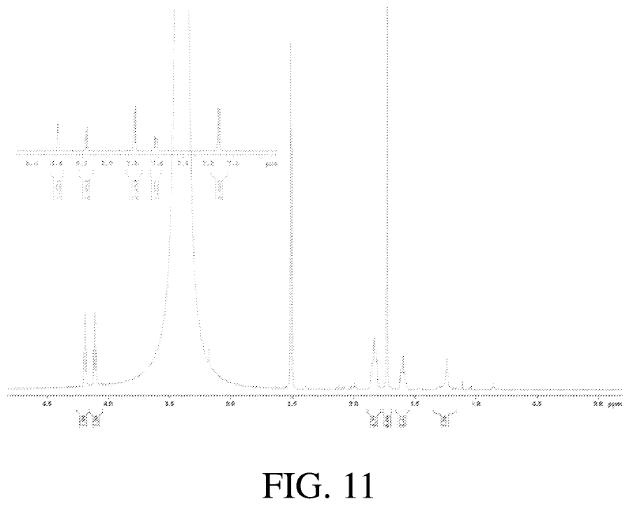

In some embodiments, Form 1 is characterized as having a ¹H NMR spectrum substantially as shown in FIG. 11.

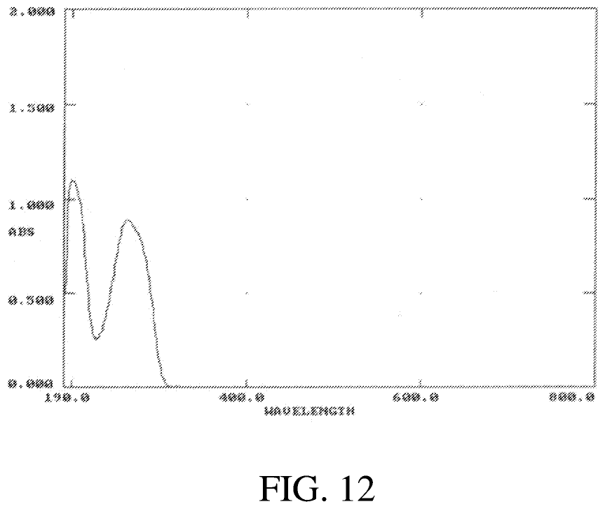

In some embodiments, Form 1 is characterized as having a DAD substantially as shown in FIG. 12.

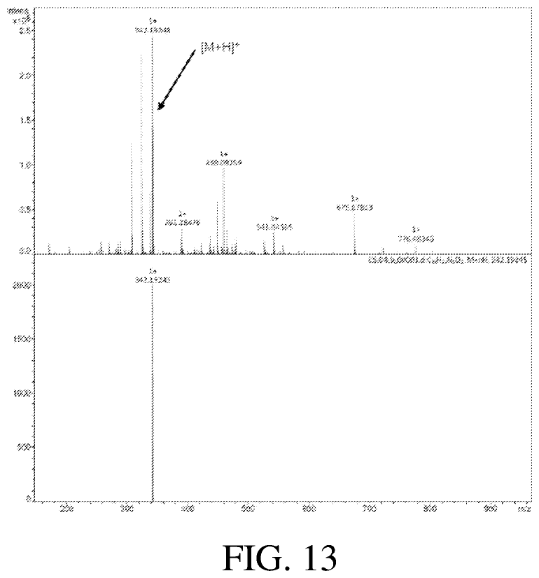

In some embodiments, Form 1 is characterized as having a LC-MS spectrum substantially as shown in FIG. 13.

In some aspects, the present invention relates to a pharmaceutical composition which comprises a therapeutically effective amount of Form 1 and a pharmaceutically acceptable excipient.

### Form 2 of Compound A Diacetate Salt

In some embodiments, the crystalline form of Compound A diacetate salt is crystalline Form 2 of Compound A diacetate salt (hereinafter referred to as “Form 2”). In some embodiments, Form 2 is a crystalline form of a hydrate of a Compound A diacetate salt. In some embodiments, Form 2 is a crystalline form of a tetrahydrate of a Compound A diacetate salt.

In some embodiments, Form 2 is characterized by an XRPD pattern substantially as set forth in FIG. 14.

In some aspects, the present invention relates to a pharmaceutical composition which comprises a therapeutically effective amount of Form 2 and a pharmaceutically acceptable excipient.

### Form 3 of Compound A Diacetate Salt

In some embodiments, the crystalline form of Compound A diacetate salt is crystalline Form 3 of Compound A diacetate salt (hereinafter referred to as “Form 3”). In some embodiments, Form 3 is a crystalline form of a THF solvate of a Compound A diacetate salt. In some embodiments, Form 3 is characterized as having an XRPD pattern having two or more diffraction peaks at two theta angles selected from 6.5564, 7.8144, 9.9506, 10.134, 10.9291, 13.0904, 14.2252, 16.57, 17.8797, 19.2866, 20.0202, 20.7913, 21.8401, 22.4817, 23.7331, 25.3538, 26.9498, 28.3475, 29.1425, 30.1636, 32.3995, and 33.3307+/−0.2 degrees two theta. In some embodiments, Form 3 is characterized as having an XRPD pattern having two or more diffraction peaks at two theta angles selected from 6.5564, 7.8144, 9.9506, 10.134, 10.9291, 13.0904, 14.2252, 16.57, 17.8797, 19.2866, 20.0202, 20.7913, 21.8401, 22.4817, 23.7331, 25.3538, 26.9498, 28.3475, 29.1425, 30.1636, 32.3995, and 33.3307+/−0.3 degrees two theta. In some embodiments, Form 3 is characterized as having an XRPD pattern having two or more diffraction peaks at two theta angles selected from 6.5564, 7.8144, 9.9506, 10.134, 10.9291, 13.0904, 14.2252, 16.57, 17.8797, 19.2866, 20.0202, 20.7913, 21.8401, 22.4817, 23.7331, 25.3538, 26.9498, 28.3475, 29.1425, 30.1636, 32.3995, and 33.3307+/−0.4 degrees two theta.

In some embodiments, Form 3 is characterized by an XRPD pattern substantially as set forth in FIG. 15.

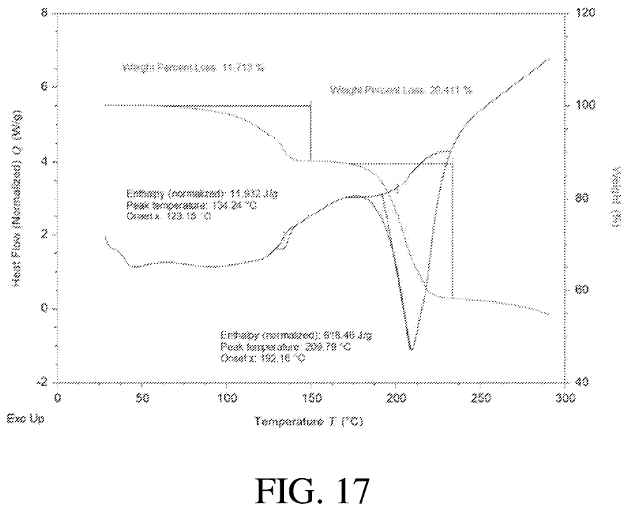

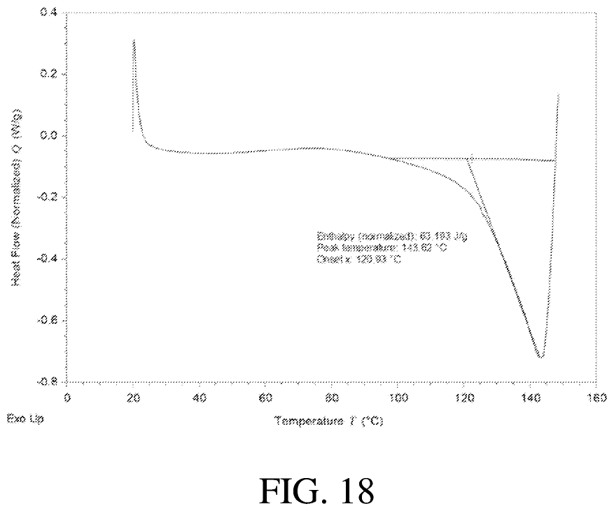

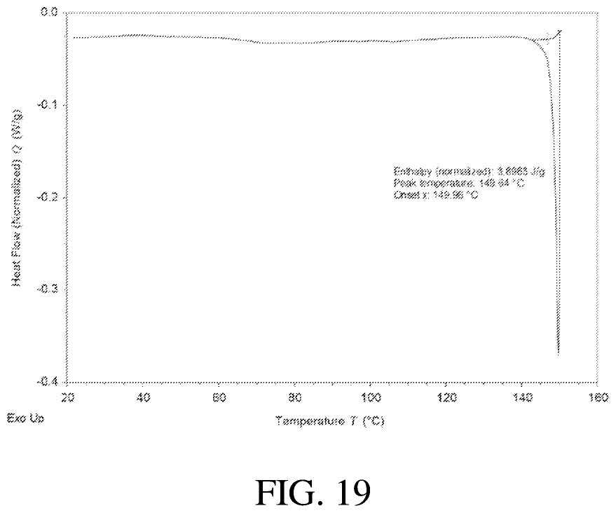

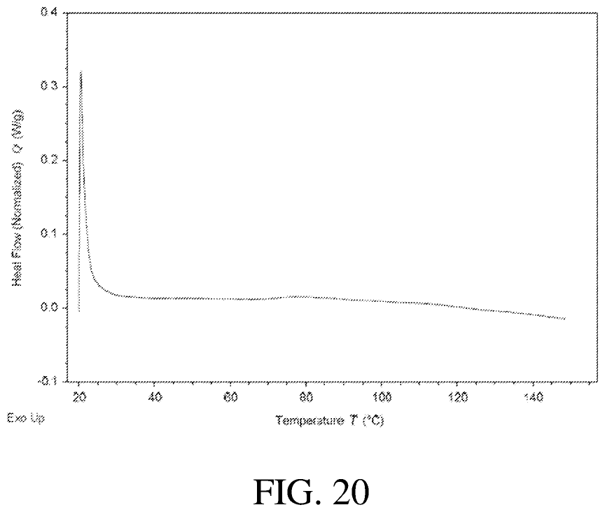

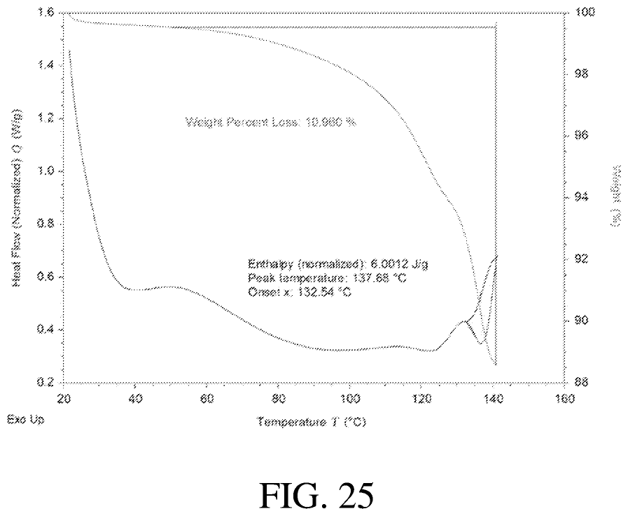

In some embodiments, Form 3 is characterized as having endotherm peaks at about 125.6° C. and/or about 197.8° C. as determined by differential scanning calorimetry (DSC). In some embodiments, Form 3 is characterized as having endotherm peaks at about 134.24° C., as determined by differential scanning calorimetry (DSC). In certain embodiments, Form 3 is characterized as having a DSC thermogram substantially as set forth in FIG. 17. In certain embodiments, Form 3 is characterized as having a DSC first heat thermogram substantially as set forth in FIG. 18. In certain embodiments, Form 3 is characterized as having a DSC cool thermogram substantially as set forth in FIG. 19. In certain embodiments, Form 3 is characterized as having a DSC second heat thermogram substantially as set forth in FIG. 20. In certain embodiments, Form 3 is characterized as having a DSC thermogram substantially as set forth in FIG. 25.

In some embodiments, Form 3 is characterized as having a weight loss of about 14.9% from about 74.0° C. to about 125.6° C., and a weight loss of about 27.9% from about 183.7° C. to 197.8° C. as determined by thermogravimetric analysis (TGA). In some embodiments, Form 3 is characterized as having a weight loss of about 29.4% at about 134.24° C. In certain embodiments, Form 3 is characterized as having a TGA graph substantially as set forth in FIG. 17.

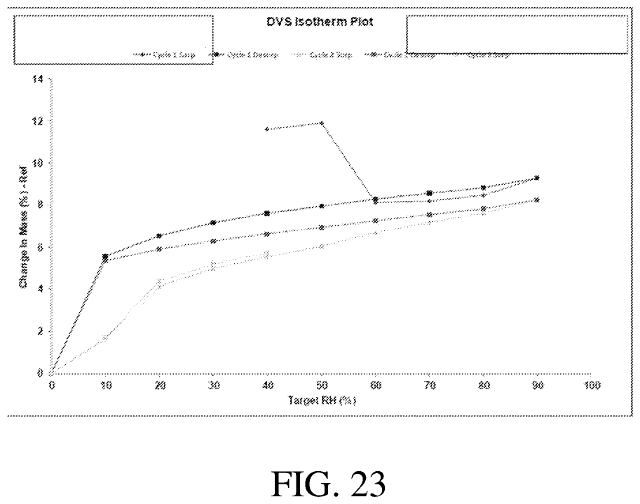

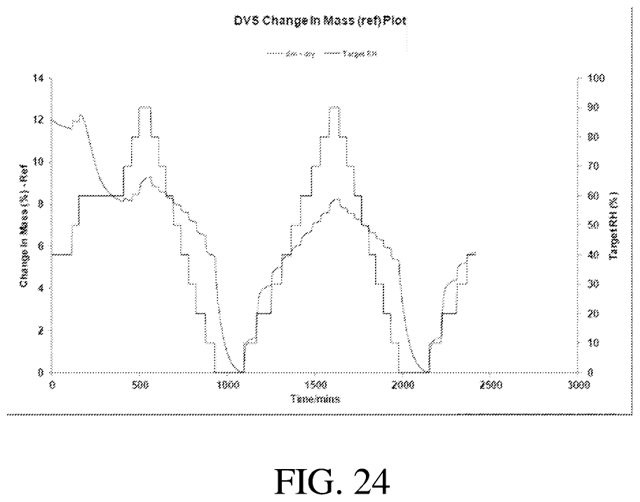

In some embodiments, Form 3 is characterized as having a DVS isotherm curve substantially as shown in FIG. 23. In some embodiments, Form 3 is characterized as having a DVS kinetic plot substantially as shown in FIG. 24.

In some embodiments, Form 3 is characterized as having an FT-IR spectrum having peaks selected from 403.8274 cm⁻¹, 410.9281 cm⁻¹, 417.8627 cm⁻¹, 427.3548 cm⁻¹, 455.8276 cm⁻¹, 497.1622 cm⁻¹, 527.8366 cm⁻¹, 613.4535 cm⁻¹, 647.0587 cm⁻¹, 667.471 cm⁻¹, 735.8367 cm⁻¹, 748.4115 cm⁻¹, 773.6263 cm⁻¹, 784.4192 cm⁻¹, 792.6738 cm⁻¹, 838.9931 cm⁻¹, 917.0699 cm⁻¹, 948.7131 cm⁻¹, 1008.2646 cm⁻¹, 1045.1361 cm⁻¹, 1113.3962 cm⁻¹, 1173.4743 cm⁻¹, 1204.3088 cm⁻¹, 1254.3655 cm⁻¹, 1275.8158 cm⁻¹, 1314.4654 cm⁻¹, 1405.5281 cm⁻¹, 1466.0255 cm⁻¹, 1496.6866 cm⁻¹, 1563.3625 cm⁻¹, 1611.6379 cm⁻¹, 1688.4984 cm⁻¹, 2780.6369 cm⁻¹, 2877.7248 cm⁻¹, 2912.3429 cm⁻¹, 2962.7601 cm⁻¹, 2997.3795 cm⁻¹, 3257.3184 cm⁻¹. In some embodiments, Form 3 is characterized as having an FT-IR spectrum substantially as shown in FIG. 25.

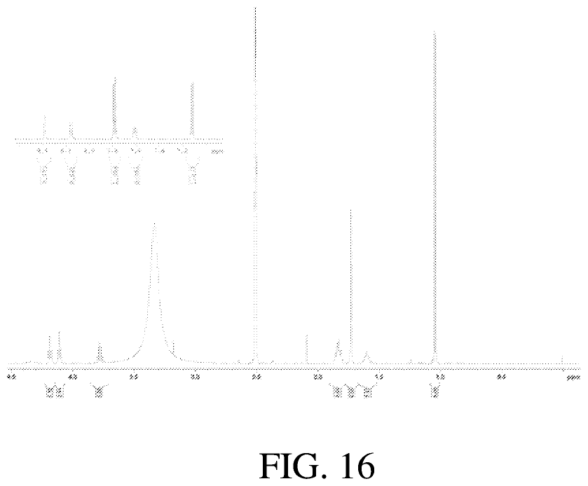

In some embodiments, Form 3 is characterized as having a ¹H NMR spectrum substantially as shown in FIG. 16.

In some aspects, the present invention relates to a pharmaceutical composition which comprises a therapeutically effective amount of Form 3 and a pharmaceutically acceptable excipient.

### Form 4 of Compound A Diacetate Salt

In some embodiments, the crystalline form of Compound A diacetate salt is crystalline Form 4 of Compound A diacetate salt (hereinafter referred to as “Form 4”). In some embodiments, Form 4 is a crystalline form of a DMA solvate of a Compound A diacetate salt.

In some embodiments, Form 4 is characterized by an XRPD pattern substantially as set forth in FIG. 26.

In some aspects, the present invention relates to a pharmaceutical composition which comprises a therapeutically effective amount of Form 4 and a pharmaceutically acceptable excipient.

### Form 5 of Compound A Diacetate Salt

In some embodiments, the crystalline form of Compound A diacetate salt is crystalline Form 5 of Compound A diacetate salt (hereinafter referred to as “Form 5”). In some embodiments, Form 5 is a crystalline form of a NMP solvate of a Compound A diacetate salt. In some embodiments, Form 5 is characterized as having an XRPD pattern having two or more diffraction peaks at two theta angles selected from 3.0955, 7.3039, 8.7822, 9.0458, 9.616, 10.0584, 10.3195, 10.6414, 10.7975, 11.5462, 11.995, 13.8128, 14.9066, 16.0215, 16.8793, 17.3302, 17.6481, 18.1419, 18.6845, 19.5967, 19.8517, 21.4041, 22.4378, 22.8546, 23.906, 24.8112, 25.2272, 26.4792, 27.8054, 28.3711, 28.6915, 29.0081, 30.4056, 30.8846, 33.1832, 34.3317, and 34.5123+/−0.2 degrees two theta. In some embodiments, Form 5 is characterized as having an XRPD pattern having two or more diffraction peaks at two theta angles selected from 3.0955, 7.3039, 8.7822, 9.0458, 9.616, 10.0584, 10.3195, 10.6414, 10.7975, 11.5462, 11.995, 13.8128, 14.9066, 16.0215, 16.8793, 17.3302, 17.6481, 18.1419, 18.6845, 19.5967, 19.8517, 21.4041, 22.4378, 22.8546, 23.906, 24.8112, 25.2272, 26.4792, 27.8054, 28.3711, 28.6915, 29.0081, 30.4056, 30.8846, 33.1832, 34.3317, and 34.5123+/−0.3 degrees two theta. In some embodiments, Form 5 is characterized as having an XRPD pattern having two or more diffraction peaks at two theta angles selected from 3.0955, 7.3039, 8.7822, 9.0458, 9.616, 10.0584, 10.3195, 10.6414, 10.7975, 11.5462, 11.995, 13.8128, 14.9066, 16.0215, 16.8793, 17.3302, 17.6481, 18.1419, 18.6845, 19.5967, 19.8517, 21.4041, 22.4378, 22.8546, 23.906, 24.8112, 25.2272, 26.4792, 27.8054, 28.3711, 28.6915, 29.0081, 30.4056, 30.8846, 33.1832, 34.3317, and 34.5123+/−0.4 degrees two theta.

In some embodiments, Form 5 is characterized by an XRPD pattern substantially as set forth in FIG. 27.

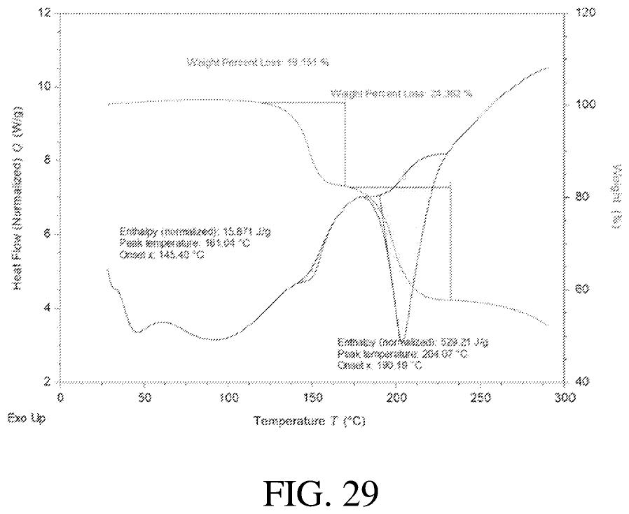

In some embodiments, Form 5 is characterized as having endotherm peaks at about 161.0° C. and/or about 204.1° C., as determined by differential scanning calorimetry (DSC). In certain embodiments, Form 5 is characterized as having a DSC thermogram substantially as set forth in FIG. 29.

In some embodiments, Form 5 is characterized as having a weight loss of about 18.2% from about 145.4° C. to about 161.0° C., and a weight loss of about 24.4% from about 190.2° C. to about 204.1° C., as determined by thermogravimetric analysis (TGA). In certain embodiments, Form 5 is characterized as having a TGA graph substantially as set forth in FIG. 29.

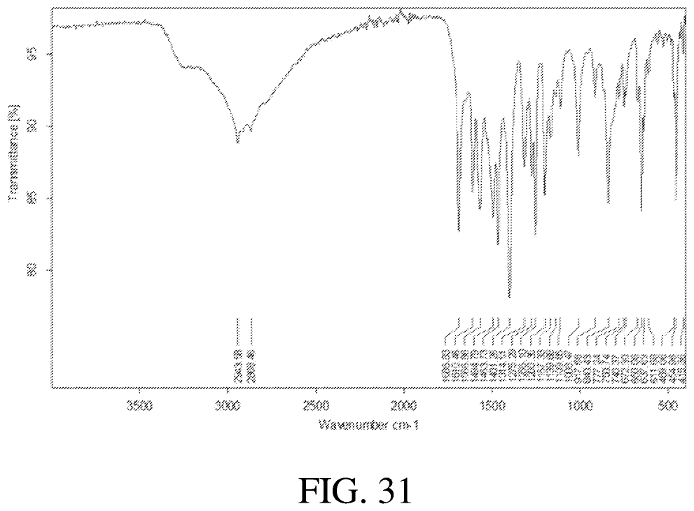

In some embodiments, Form 5 is characterized as having an FT-IR spectrum having peaks selected from 454.9594 cm⁻¹, 469.0604 cm⁻¹, 611.6827 cm⁻¹, 637.6883 cm⁻¹, 650.0553 cm⁻¹, 672.3002 cm⁻¹, 740.3731 cm⁻¹. 750.7427 cm⁻¹, 777.2387 cm⁻¹, 840.4285 cm⁻¹, 917.5764 cm⁻¹, 1008.4717 cm⁻¹, 1109.8542 cm⁻¹, 1139.6816 cm⁻¹. 1167.3318 cm⁻¹, 1200.3449 cm⁻¹, 1256.0996 cm⁻¹, 1275.2878 cm⁻¹, 1314.5075 cm⁻¹, 1401.2548 cm⁻¹, 1463.7291 cm⁻¹, 1494.7887 cm⁻¹, 1566.8599 cm⁻¹, 1610.4636 cm⁻¹, 1686.825 cm⁻¹, 2868.4556 cm⁻¹, and 2943.5756 cm⁻¹. In some embodiments, Form 5 is characterized as having an FT-IR spectrum substantially as shown in FIG. 31.

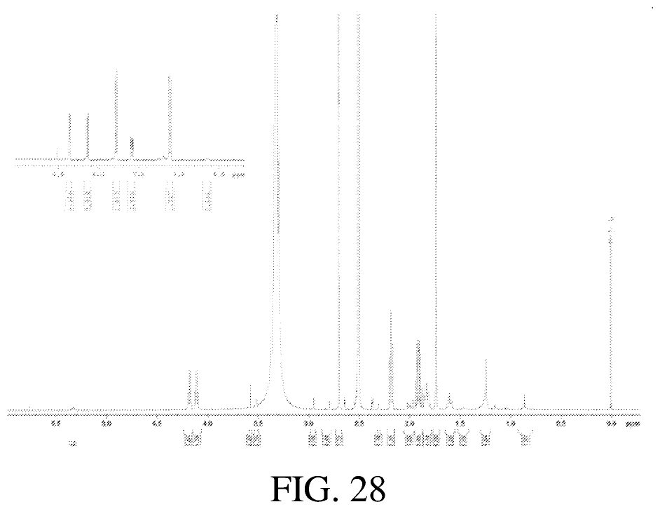

In some embodiments, Form 5 is characterized as having a ¹H NMR spectrum substantially as shown in FIG. 28.

In some aspects, the present invention relates to a pharmaceutical composition which comprises a therapeutically effective amount of Form 5 and a pharmaceutically acceptable excipient.

### Form 6 of Compound A Diacetate Salt

In some embodiments, the crystalline form of Compound A diacetate salt is crystalline Form 6 of Compound A diacetate salt (hereinafter referred to as “Form 6”). In some embodiments, Form 6 is characterized as having an XRPD pattern having two or more diffraction peaks at two theta angles selected from 5.6904, 7.4999, 9.0887, 9.5349, 11.4096, 12.5161, 17.054, 17.5331, 18.2714, 18.4887, 20.1801, 21.8337, 22.4764, 24.7984, 26.0531, 26.5658, 31.3369, and 32.5215+/−0.2 degrees two theta. In some embodiments, Form 6 is characterized as having an XRPD pattern having two or more diffraction peaks at two theta angles selected from 5.6904, 7.4999, 9.0887, 9.5349, 11.4096, 12.5161, 17.054, 17.5331, 18.2714, 18.4887, 20.1801, 21.8337, 22.4764, 24.7984, 26.0531, 26.5658, 31.3369, and 32.5215+/−0.3 degrees two theta. In some embodiments, Form 6 is characterized as having an XRPD pattern having two or more diffraction peaks at two theta angles selected from 5.6904, 7.4999, 9.0887, 9.5349, 11.4096, 12.5161, 17.054, 17.5331, 18.2714, 18.4887, 20.1801, 21.8337, 22.4764, 24.7984, 26.0531, 26.5658, 31.3369, and 32.5215+/−0.4 degrees two theta.

In some embodiments, Form 6 is characterized by an XRPD pattern substantially as set forth in FIG. 32.

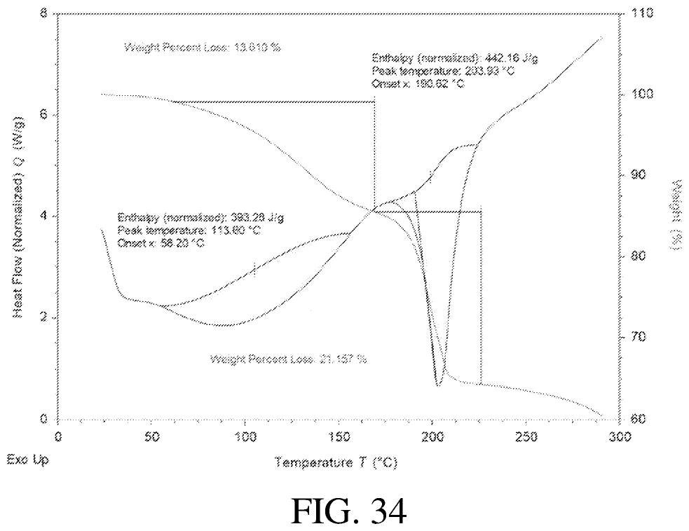

In some embodiments, Form 6 is characterized as having endotherm peaks at about 113.6° C. and/or about 203.9° C., as determined by differential scanning calorimetry (DSC). In certain embodiments, Form 6 is characterized as having a DSC thermogram substantially as set forth in FIG. 34.

In some embodiments, Form 6 is characterized as having a weight loss of about 13.6% from about 58.2° C. to about 113.6° C., and a weight loss of about 21.2% from about 190.6° C. to about 203.9° C., as determined by thermogravimetric analysis (TGA). In certain embodiments, Form 6 is characterized as having a TGA graph substantially as set forth in FIG. 34.

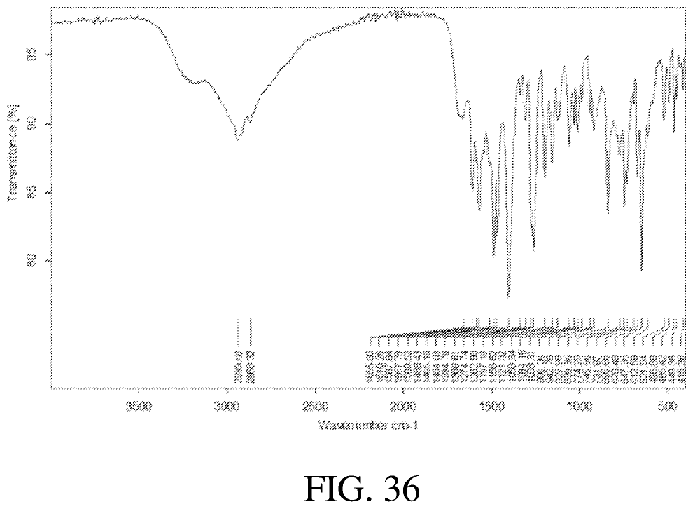

In some embodiments, Form 6 is characterized as having an FT-IR spectrum having peaks selected from 415.3786 cm⁻¹, 449.3573 cm⁻¹, 465.4179 cm⁻¹, 495.8008 cm⁻¹, 521.5347 cm⁻¹, 612.5942 cm⁻¹, 647.363 cm⁻¹, 670.4807 cm⁻¹, 695.6592 cm⁻¹, 731.8654 cm⁻¹, 745.9633 cm⁻¹, 774.2919 cm⁻¹, 839.3638 cm⁻¹. 922.9869 cm⁻¹ 942.7633 cm⁻¹, 986.3551 cm⁻¹, 1008.8106 cm⁻¹, 1034.18 cm⁻¹, 1058.8405 cm⁻¹, 1121.3221 cm⁻¹, cm⁻¹, 1156.6177 cm⁻¹, 1197.1751 cm⁻¹, 1262.8981 cm⁻¹, 1274.7356 cm⁻¹, 1306.6066 cm⁻¹, 1334.7588 cm⁻¹, 1404.0304 cm⁻¹, 1465.1639 cm⁻¹, 1488.4329 cm⁻¹, 1509.518 cm⁻¹, 1567.7745 cm⁻¹, 1587.841 cm⁻¹, 1610.2619 cm⁻¹, 1655.8039 cm⁻¹, 2868.3153 cm⁻¹, and 2939.6823 cm⁻¹. In some embodiments, Form 6 is characterized as having an FT-IR spectrum substantially as shown in FIG. 36.

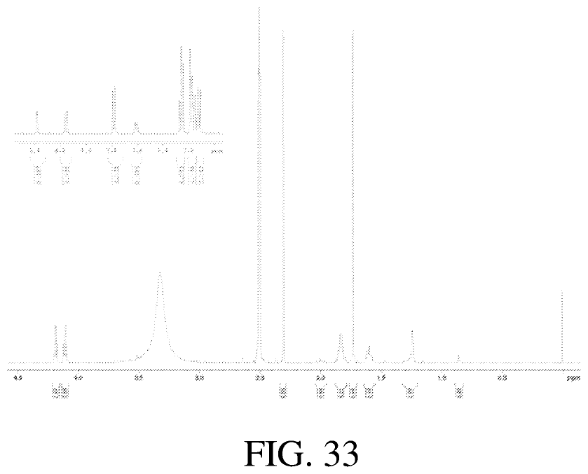

In some embodiments, Form 6 is characterized as having a ¹H NMR spectrum substantially as shown in FIG. 33.

In some aspects, the present invention relates to a pharmaceutical composition which comprises a therapeutically effective amount of Form 6 and a pharmaceutically acceptable excipient.

### Form 7 of Compound A Diacetate Salt

In some embodiments, the crystalline form of Compound A diacetate salt is crystalline Form 7 of Compound A diacetate salt (hereinafter referred to as “Form 7”). In some embodiments, Form 7 is a crystalline form of a hydrate of a Compound A diacetate salt. In some embodiments, Form 7 is a crystalline form of a monohydrate of Compound A diacetate salt. In some embodiments, Form 7 is characterized as having an XRPD pattern having two or more diffraction peaks at two theta angles selected from 5.1087, 5.6134, 10.1362, 10.4265, 10.6607, 11.3637, 11.5103, 11.6747, 12.7372, 13.0192, 14.3972, 14.8513, 15.3999, 16.4208, 16.6467, 16.7178, 17.7524, 17.9449, 18.4299, 18.5083, 18.7792, 20.3597, 20.5204, 21.0321, 21.8055, 22.0923, 22.4226, 22.7017, 22.8773, 23.4679, 23.7026, 24.1586, 24.7744, 25.2874, 26.2699, 27.2182, 27.4644, 27.9591, 28.2391, 29.1359, 29.5639, 30.1954, 31.1076, 31.4344, 31.7841, 33.1681, 33.9234, and 34.7491+/−0.2 degrees two theta. In some embodiments, Form 7 is characterized as having an XRPD pattern having two or more diffraction peaks at two theta angles selected from 5.1087, 5.6134, 10.1362, 10.4265, 10.6607, 11.3637, 11.5103, 11.6747, 12.7372, 13.0192, 14.3972, 14.8513, 15.3999, 16.4208, 16.6467, 16.7178, 17.7524, 17.9449, 18.4299, 18.5083, 18.7792, 20.3597, 20.5204, 21.0321, 21.8055, 22.0923, 22.4226, 22.7017, 22.8773, 23.4679, 23.7026, 24.1586, 24.7744, 25.2874, 26.2699, 27.2182, 27.4644, 27.9591, 28.2391, 29.1359, 29.5639, 30.1954, 31.1076, 31.4344, 31.7841, 33.1681, 33.9234, and 34.7491+/−0.3 degrees two theta. In some embodiments, Form 7 is characterized as having an XRPD pattern having two or more diffraction peaks at two theta angles selected from 5.1087, 5.6134, 10.1362, 10.4265, 10.6607, 11.3637, 11.5103, 11.6747, 12.7372, 13.0192, 14.3972, 14.8513, 15.3999, 16.4208, 16.6467, 16.7178, 17.7524, 17.9449, 18.4299, 18.5083, 18.7792, 20.3597, 20.5204, 21.0321, 21.8055, 22.0923, 22.4226, 22.7017, 22.8773, 23.4679, 23.7026, 24.1586, 24.7744, 25.2874, 26.2699, 27.2182, 27.4644, 27.9591, 28.2391, 29.1359, 29.5639, 30.1954, 31.1076, 31.4344, 31.7841, 33.1681, 33.9234, and 34.7491+/−0.4 degrees two theta.

In some embodiments, Form 7 is characterized by an XRPD pattern substantially as set forth in FIG. 37.

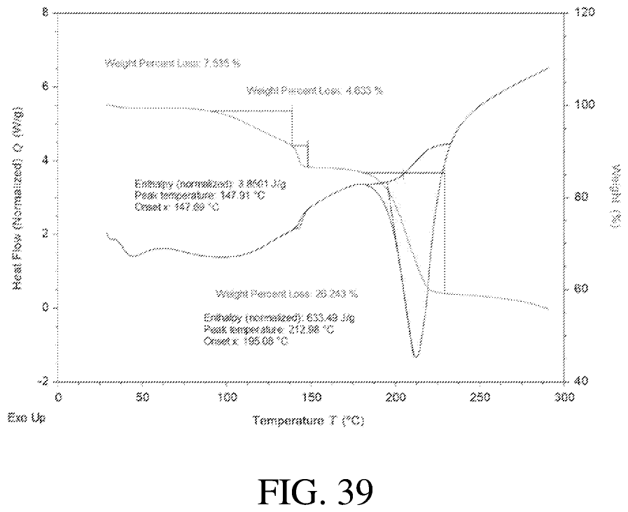

In some embodiments, Form 7 is characterized as having endotherm peaks at about 147.9° C. and/or about 213.0° C., as determined by differential scanning calorimetry (DSC). In certain embodiments, Form 7 is characterized as having a DSC thermogram substantially as set forth in FIG. 39.

In some embodiments, Form 7 is characterized as having a gradual 7.5% mass loss prior to a sharp 4.6% mass loss from about 147.7° C. to about 147.9° C. and a weight loss of about 26.2% from about 195.1° C. to about 213.0° C., as determined by thermogravimetric analysis (TGA). In some embodiments, Form 7 is characterized as having a 4.6% mass loss from about 147.7° C. to about 147.9° C. as measured by TGA. In certain embodiments, Form 7 is characterized as having a TGA graph substantially as set forth in FIG. 39.

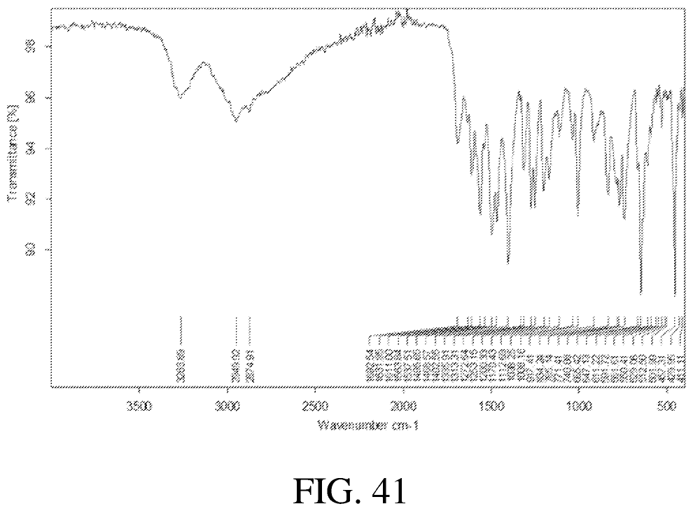

In some embodiments, Form 7 is characterized as having an FT-IR spectrum having peaks selected from 411.1117 cm⁻¹, 429.951 cm⁻¹, 457.3076 cm⁻¹, 501.991 cm⁻¹, 512.5004 cm⁻¹, 529.0548 cm⁻¹, 550.4058 cm⁻¹ 561.6064 cm⁻¹ 591.7189 cm⁻¹, 611.2152 cm⁻¹, 647.1254 cm⁻¹, 665.4149 cm⁻¹. 740.8762 cm⁻¹ 771.4092 cm⁻¹, 785.1443 cm⁻¹, 834.2445 cm⁻¹, 917.4091 cm⁻¹, 1008.155 cm⁻¹ 1038.2526 cm⁻¹, 1112.6934 cm⁻¹, 1170.4312 cm⁻¹, 1200.3311 cm⁻¹ 1253.1489 cm⁻¹, 1272.6386 cm⁻¹, 1313.3134 cm⁻¹, 1335.9096 cm⁻¹, 1402.5501 cm⁻¹, 1468.5722 cm⁻¹, 1495.6468 cm⁻¹, 1537.5083 cm⁻¹, 1563.8372 cm⁻¹, 1610.9948 cm⁻¹, 1631.9449 cm⁻¹, 1692.5437 cm⁻¹, 2874.9139 cm⁻¹, 2949.921 cm⁻¹, and 3263.8881 cm⁻¹. In some embodiments, Form 7 is characterized as having an FT-IR spectrum substantially as shown in FIG. 41.

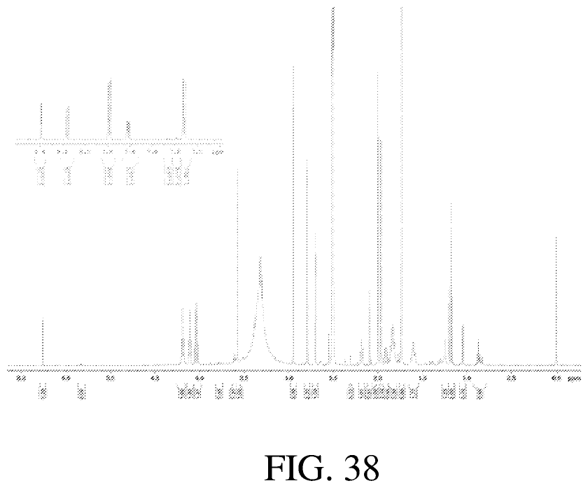

In some embodiments, Form 7 is characterized as having a ¹H NMR spectrum substantially as shown in FIG. 38.

In some aspects, the present invention relates to a pharmaceutical composition which comprises a therapeutically effective amount of Form 7 and a pharmaceutically acceptable excipient.

### Form 8 of Compound A Diacetate Salt

In some embodiments, the crystalline form of Compound A diacetate salt is crystalline Form 8 of Compound A diacetate salt (hereinafter referred to as “Form 8”). In some embodiments, Form 8 is a crystalline form of an ethanol solvate of a Compound A diacetate salt. In some embodiments, Form 8 is characterized as having an XRPD pattern having two or more diffraction peaks at two theta angles selected from 5.5331, 9.2347, 10.4522, 11.0878, 12.1465, 13.9753, 14.9568, 16.7343, 17.8535, 18.4906, 19.0176, 20.2017, 21.5889, 21.9169, 22.5325, 23.4194, 24.04, 24.625, 24.8346, 25.5526, 25.9083, 26.4698, 27.6197, 28.6765, 28.7907, 30.0882, 30.8638, 31.6522, 33.6849+/−0.2 degrees two theta. In some embodiments, Form 8 is characterized as having an XRPD pattern having two or more diffraction peaks at two theta angles selected from 5.5331, 9.2347, 10.4522, 11.0878, 12.1465, 13.9753, 14.9568, 16.7343, 17.8535, 18.4906, 19.0176, 20.2017, 21.5889, 21.9169, 22.5325, 23.4194, 24.04, 24.625, 24.8346, 25.5526, 25.9083, 26.4698, 27.6197, 28.6765, 28.7907, 30.0882, 30.8638, 31.6522, 33.6849+/−0.3 degrees two theta. In some embodiments, Form 8 is characterized as having an XRPD pattern having two or more diffraction peaks at two theta angles selected from 5.5331, 9.2347, 10.4522, 11.0878, 12.1465, 13.9753, 14.9568, 16.7343, 17.8535, 18.4906, 19.0176, 20.2017, 21.5889, 21.9169, 22.5325, 23.4194, 24.04, 24.625, 24.8346, 25.5526, 25.9083, 26.4698, 27.6197, 28.6765, 28.7907, 30.0882, 30.8638, 31.6522, 33.6849+/−0.4 degrees two theta.

In some embodiments, Form 8 is characterized by an XRPD pattern substantially as set forth in FIG. 42.

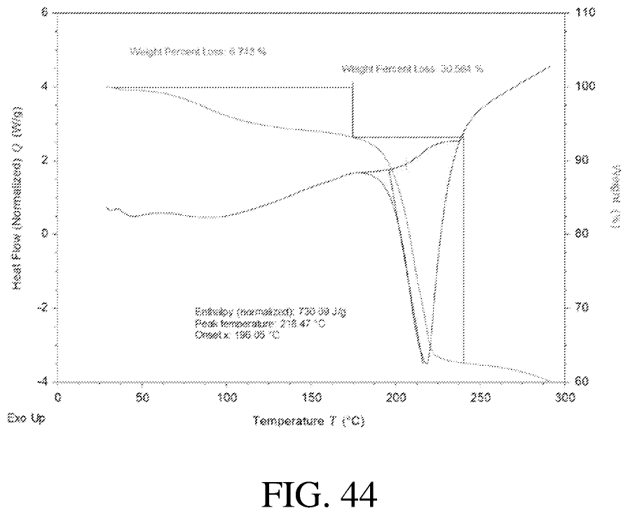

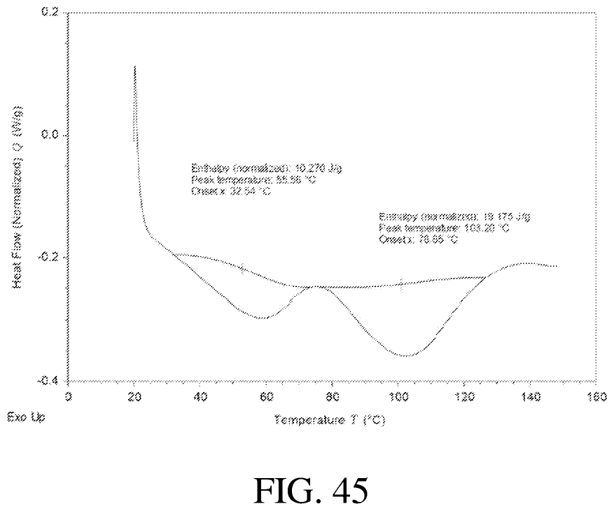

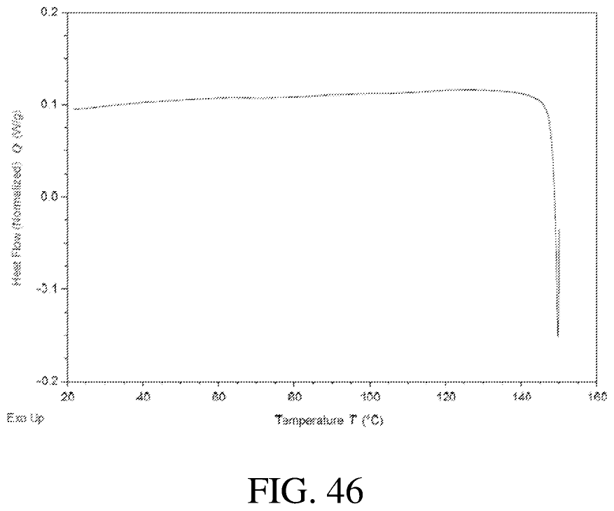

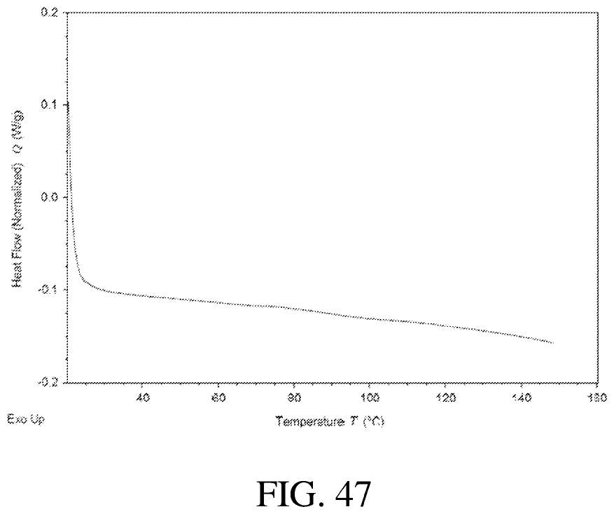

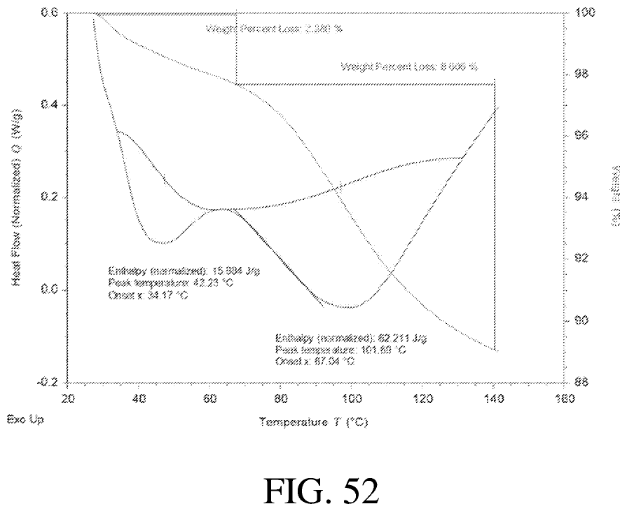

In some embodiments, Form 8 is characterized as having endotherm peaks at about 203.7° C., as determined by differential scanning calorimetry (DSC). In certain embodiments, Form 8 is characterized as having a DSC thermogram substantially as set forth in FIG. 44. In certain embodiments, Form 8 is characterized as having a DSC first heat thermogram substantially as set forth in FIG. 45. In certain embodiments, Form 8 is characterized as having a DSC cool thermogram substantially as set forth in FIG. 46. In certain embodiments, Form 8 is characterized as having a DSC second heat thermogram substantially as set forth in FIG. 47. In certain embodiments, Form 8 is characterized as having a DSC thermogram substantially as set forth in FIG. 52.

In some embodiments, Form 8 is characterized as having a weight loss of about 28.6% from about 186.0° C. to about 203.7° C., as determined by thermogravimetric analysis (TGA). In certain embodiments, Form 8 is characterized as having a TGA graph substantially as set forth in FIG. 44.

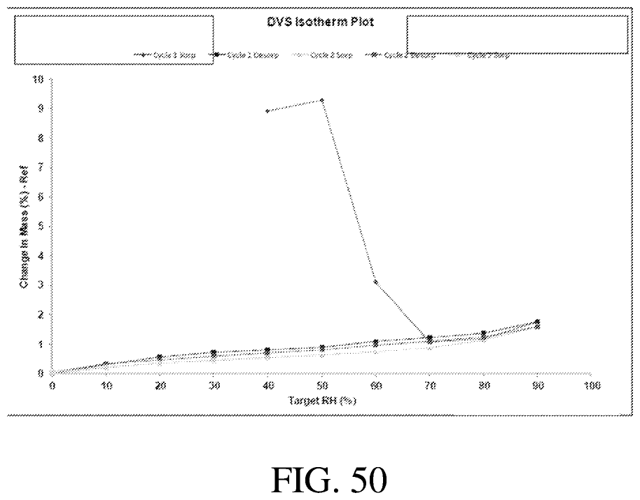

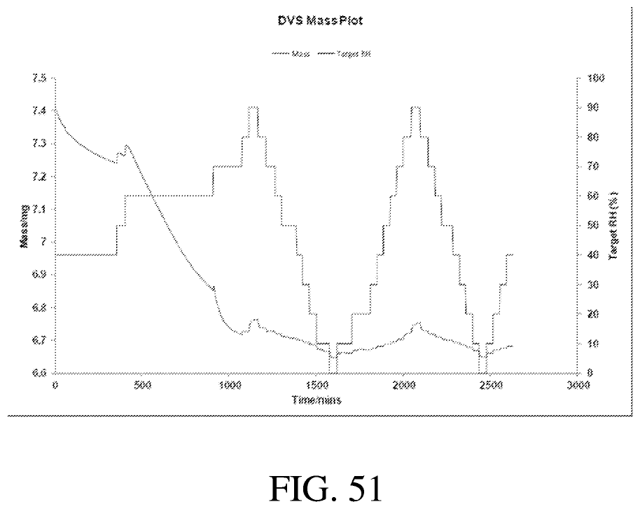

In some embodiments, Form 8 is characterized as having a DVS isotherm curve substantially as shown in FIG. 50. In some embodiments, Form 8 is characterized as having a DVS kinetic plot substantially as shown in FIG. 51.

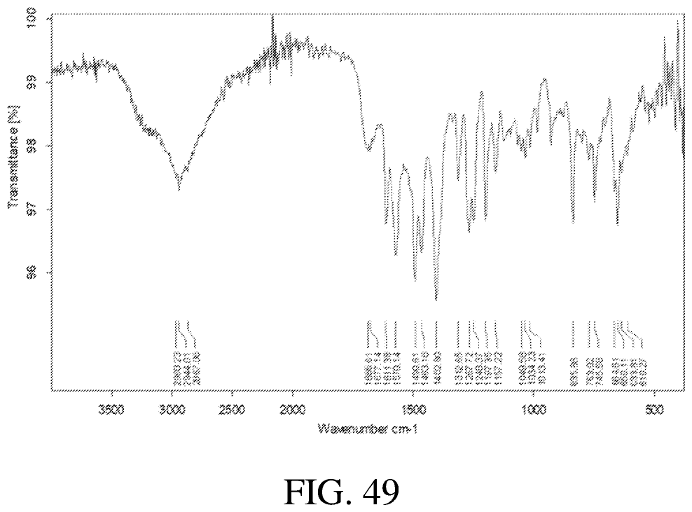

In some embodiments. Form 8 is characterized as having an FT-IR spectrum having peaks selected from 610.2657 cm⁻¹, 633.8131 cm⁻¹, 650.1075 cm⁻¹, 664.611 cm⁻¹, 745.5844 cm⁻¹, 769.0208 cm⁻¹, 835.8804 cm⁻¹, 1013.4095 cm⁻¹, 1034.2339 cm⁻¹, 1049.5794 cm⁻¹, 1157.2206 cm⁻¹, 1197.3449 cm⁻¹, 1248.3703 cm⁻¹, 1267.7224 cm⁻¹, 1312.6521 cm⁻¹, 1402.7999 cm⁻¹, 1463.1612 cm⁻¹, 1490.611 cm⁻¹, 1570.1416 cm⁻¹, 1611.382 cm⁻¹, 1677.1372 cm⁻¹, 1686.6121 cm⁻¹, 2867.0613 cm⁻¹, 2944.0111 cm⁻¹, and 2960.2257 cm⁻¹. In some embodiments, Form 8 is characterized as having an FT-IR spectrum substantially as shown in FIG. 49.

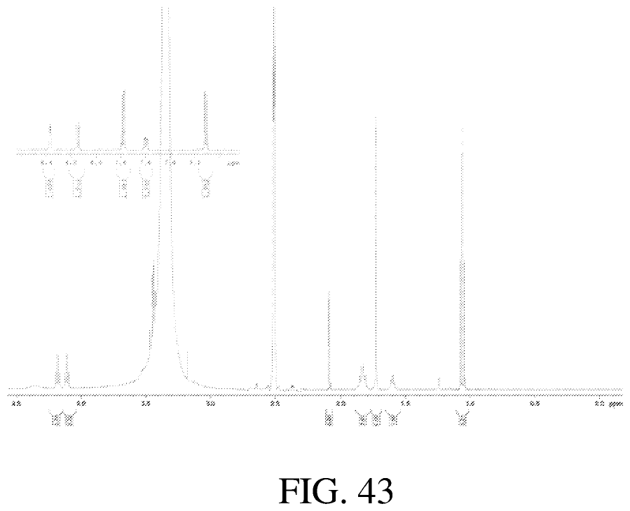

In some embodiments, Form 8 is characterized as having a ¹H NMR spectrum substantially as shown in FIG. 43.

In some aspects, the present invention relates to a pharmaceutical composition which comprises a therapeutically effective amount of Form 8 and a pharmaceutically acceptable excipient.

### Form 9 of Compound A Diacetate Salt

In some embodiments, the crystalline form of Compound A diacetate salt is crystalline Form 9 of Compound A diacetate salt (hereinafter referred to as “Form 9”). In some embodiments, Form 9 is a crystalline form of a hydrate of a Compound A diacetate salt. In some embodiments, Form 9 is characterized as having an XRPD pattern having two or more diffraction peaks at two theta angles selected from 3.9466, 4.9134, 6.3449, 7.0644, 10.3976, 11.4501, 12.5469, 12.7439, 13.6904, 14.0455, 14.1993, 14.8142, 15.0272, 16.0564, 17.0908, 17.4764, 17.9115, 18.6031, 19.1762, 19.3881, 19.8051, 20.0332, 20.8087, 21.3351, 21.4789, 23.0366, 23.2768, 23.6282, 24.1482, 24.602, 25.0239, 25.2132, 26.0594, 26.5702, 27.4322, 27.6305, 28.1236, 28.5678, 29.0804, 29.6687, 30.1387, 30.9207, 31.4008, 32.3553, and 34.516+/−0.2 degrees two theta. In some embodiments, Form 9 is characterized as having an XRPD pattern having two or more diffraction peaks at two theta angles selected from 3.9466, 4.9134, 6.3449, 7.0644, 10.3976, 11.4501, 12.5469, 12.7439, 13.6904, 14.0455, 14.1993, 14.8142, 15.0272, 16.0564, 17.0908, 17.4764, 17.9115, 18.6031, 19.1762, 19.3881, 19.8051, 20.0332, 20.8087, 21.3351, 21.4789, 23.0366, 23.2768, 23.6282, 24.1482, 24.602, 25.0239, 25.2132, 26.0594, 26.5702, 27.4322, 27.6305, 28.1236, 28.5678, 29.0804, 29.6687, 30.1387, 30.9207, 31.4008, 32.3553, and 34.516+/−0.3 degrees two theta. In some embodiments, Form 9 is characterized as having an XRPD pattern having two or more diffraction peaks at two theta angles selected from 3.9466, 4.9134, 6.3449, 7.0644, 10.3976, 11.4501, 12.5469, 12.7439, 13.6904, 14.0455, 14.1993, 14.8142, 15.0272, 16.0564, 17.0908, 17.4764, 17.9115, 18.6031, 19.1762, 19.3881, 19.8051, 20.0332, 20.8087, 21.3351, 21.4789, 23.0366, 23.2768, 23.6282, 24.1482, 24.602, 25.0239, 25.2132, 26.0594, 26.5702, 27.4322, 27.6305, 28.1236, 28.5678, 29.0804, 29.6687, 30.1387, 30.9207, 31.4008, 32.3553, and 34.516+/−0.4 degrees two theta.

In some embodiments, Form 9 is characterized by an XRPD pattern substantially as set forth in FIG. 53.

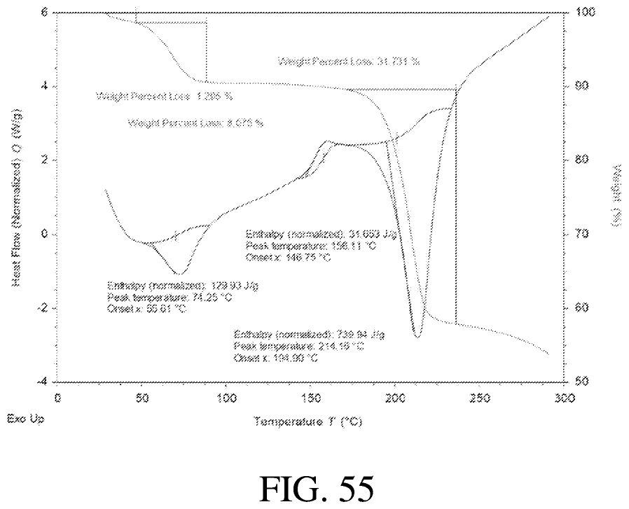

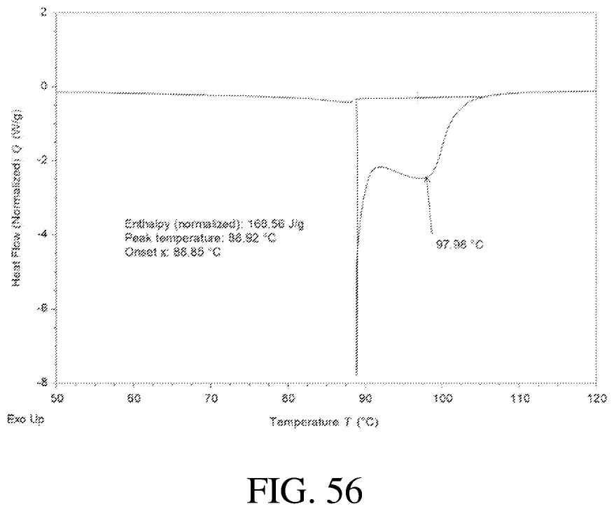

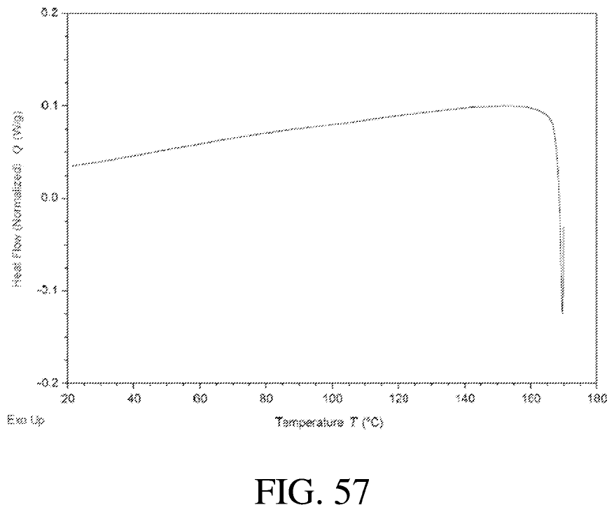

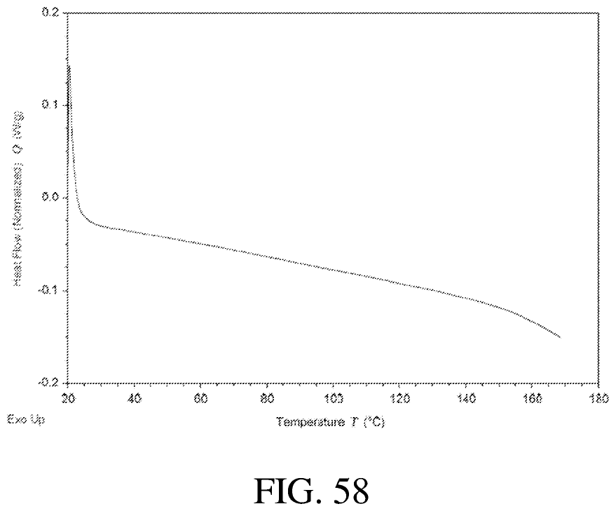

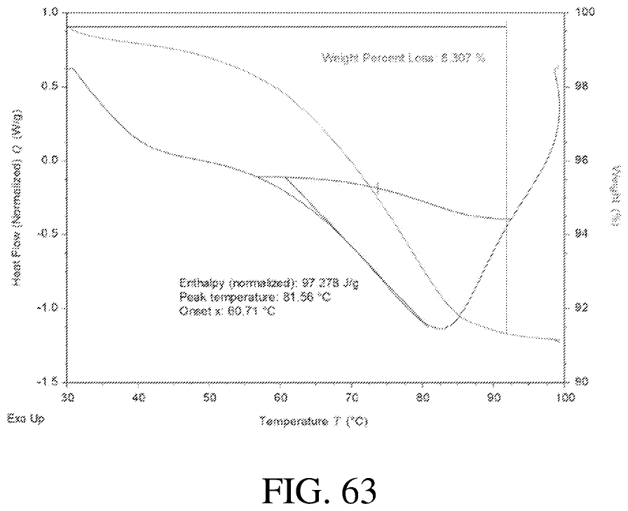

In some embodiments, Form 9 is characterized as having endotherm peaks at about 74.25° C. and/or about 206.7° C. as determined by differential scanning calorimetry (DSC). In certain embodiments, Form 9 is characterized as having a DSC thermogram substantially as set forth in FIG. 55. In certain embodiments, Form 9 is characterized as having a DSC first heat thermogram substantially as set forth in FIG. 56. In certain embodiments, Form 9 is characterized as having a DSC cool thermogram substantially as set forth in FIG. 57. In certain embodiments, Form 9 is characterized as having a DSC second heat thermogram substantially as set forth in FIG. 58. In certain embodiments, Form 9 is characterized as having a DSC thermogram substantially as set forth in FIG. 63.

In some embodiments, Form 9 is characterized as having a weight loss of about 6.7% at about 50° C. and a weight loss of about 30.9% from about 191.6° C. to about 206.7° C., as determined by thermogravimetric analysis (TGA). In certain embodiments, Form 9 is characterized as having a TGA graph substantially as set forth in FIG. 55.

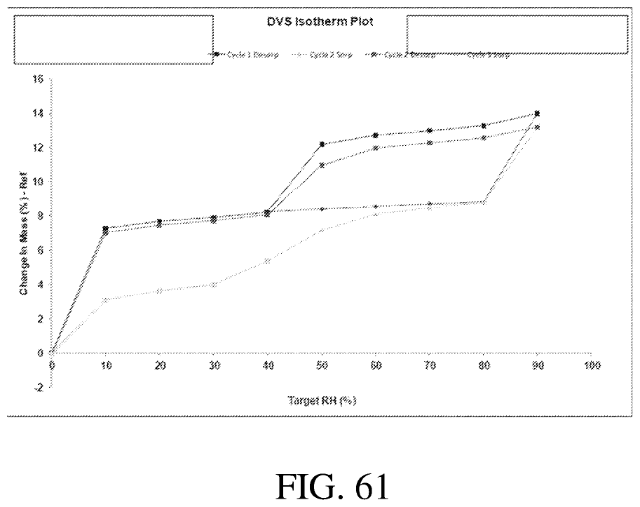

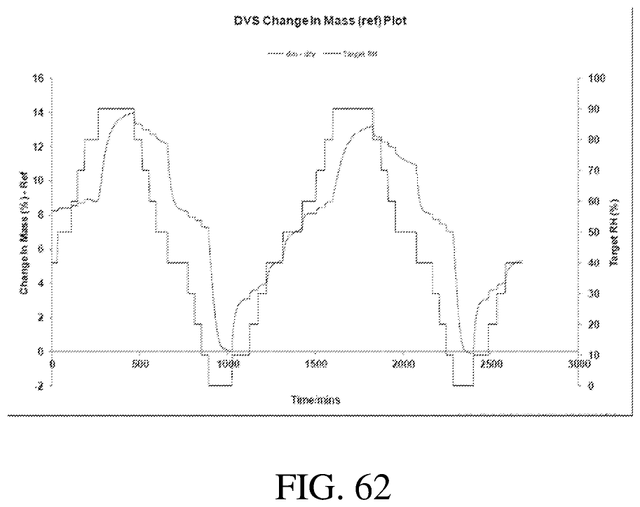

In some embodiments, Form 9 is characterized as having a DVS isotherm curve substantially as shown in FIG. 61. In some embodiments, Form 9 is characterized as having a DVS kinetic plot substantially as shown in FIG. 62.

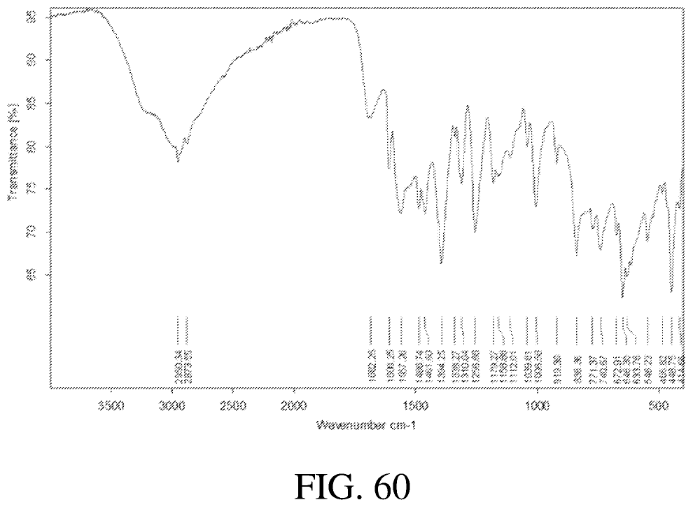

In some embodiments, Form 9 is characterized as having an FT-IR spectrum having peaks selected from 414.6559 cm⁻¹, 448.747 cm⁻¹, 486.815 cm⁻¹, 546.2329 cm⁻¹, 633.7561 cm⁻¹, 648.299 cm⁻¹, 672.9115 cm⁻¹, 740.6708 cm⁻¹, 771.3666 cm⁻¹, 838.3607 cm⁻¹, 919.3036 cm⁻¹, 1006.5771 cm⁻¹, 1039.8083 cm⁻¹, 1112.0083 cm⁻¹, 1158.8791 cm⁻¹, 1179.2694 cm⁻¹, 1256.6641 cm⁻¹, 1310.0412 cm⁻¹, 1338.2741 cm⁻¹, 1394.2529 cm⁻¹, 1461.6026 cm⁻¹, 1486.7422 cm⁻¹, 1557.2577 cm⁻¹, 1608.2499 cm⁻¹, 1682.2509 cm⁻¹, 2873.5453 cm⁻¹, and 2950.3382 cm⁻¹. In some embodiments, Form 9 is characterized as having an FT-IR spectrum substantially as shown in FIG. 60.

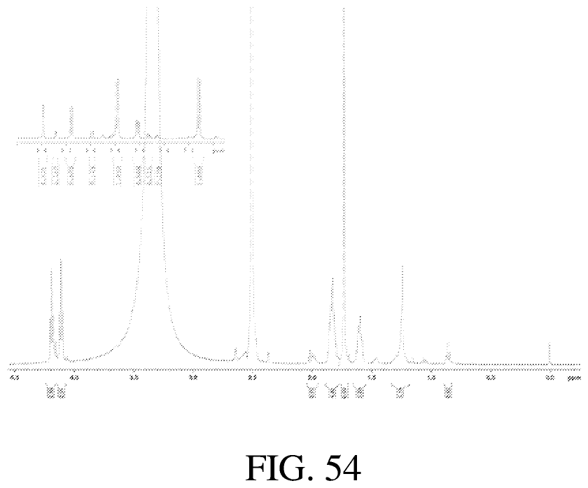

In some embodiments, Form 9 is characterized as having a ¹H NMR spectrum substantially as shown in FIG. 54.

In some aspects, the present invention relates to a pharmaceutical composition which comprises a therapeutically effective amount of Form 9 and a pharmaceutically acceptable excipient.

### Form 10 of Compound A Diacetate Salt

In some embodiments, the crystalline form of Compound A diacetate salt is crystalline Form 10 of Compound A diacetate salt (hereinafter referred to as “Form 10”).

In some aspects, the present invention relates to a pharmaceutical composition which comprises a therapeutically effective amount of Form 10 and a pharmaceutically acceptable excipient.

### Form 11 of Compound A Diacetate Salt

In some embodiments, the crystalline form of Compound A diacetate salt is crystalline Form 11 of Compound A diacetate salt (hereinafter referred to as “Form 11”).

In some embodiments, Form 11 is characterized by an XRPD pattern substantially as set forth in FIG. 64.

In some aspects, the present invention relates to a pharmaceutical composition which comprises a therapeutically effective amount of Form 11 and a pharmaceutically acceptable excipient.

### Form 12 of Compound A Phosphate Salt

In some embodiments, the crystalline form of Compound A phosphate salt is crystalline Form 12 of Compound A phosphate salt (hereinafter referred to as “Form 12”).

In some embodiments, Form 12 is characterized by an XRPD pattern substantially as set forth in FIG. 65.

In some aspects, the present invention relates to a pharmaceutical composition which comprises a therapeutically effective amount of Form 12 and a pharmaceutically acceptable excipient.

### Form 13 of Compound A Diacetate Salt

In some embodiments, the crystalline form of Compound A diacetate salt is crystalline Form 13 of Compound A diacetate salt (hereinafter referred to as “Form 13”).

In some embodiments, Form 13 is characterized by an XRPD pattern substantially as set forth in FIG. 66.

In some aspects, the present invention relates to a pharmaceutical composition which comprises a therapeutically effective amount of Form 13 and a pharmaceutically acceptable excipient.

### Purity

In some embodiments, the crystalline salt of Compound A or solvate thereof produced by the methods described herein has a purity level of at least about 85%, at least about 90%, at least about 91%, at least about 92%, at least about 93%, at least about 94%, at least about 95%, at least about 96%, at least about 97%, at least 98%, or at least about 99% as determined by ultra-performance liquid chromatography (UPLC), high-performance liquid chromatography (HPLC), or other appropriate methods. In some embodiments, the crystalline salt of Compound A or solvate thereof has a purity level of between about 90% and 100%. In some embodiments, the crystalline salt of Compound A or solvate thereof has a purity level of at least about 95.0%, about 96.0%, about 97.0%, about 98.0%, about 99.0%, about 99.1%, about 99.2%, about 99.3%, about 99.4%, about 99.5%, about 99.6%, about 99.7%, about 99.8%, or about 99.9%, including any amount in between and fractions thereof. In other embodiments, the crystalline salt of Compound A or solvate thereof has a purity level of between about 95.0% and 99.9%. In some embodiments, the crystalline salt of Compound A or solvate thereof has a purity level of at least 95%. In some embodiments, the crystalline salt of Compound A or solvate thereof has a purity level of at least 96%. In some embodiments, the crystalline salt of Compound A or solvate thereof has a purity level of at least 97%. In some embodiments, the crystalline salt of Compound A or solvate thereof has a purity level of at least 98%. In some embodiments, the crystalline salt of Compound A or solvate thereof has a purity level of at least 99%. In some embodiments, the crystalline salt of Compound A or solvate thereof has a purity level of at least 99.5%.

In some embodiments, Form 1 of Compound A has a purity level of at least about 95.0%, about 96.0%, about 97.0%, about 98.0%, about 99.0%, about 99.10%, about 99.2%, about 99.3%, about 99.4%, about 99.5%, about 99.6%, about 99.7%, about 99.8%, or about 99.9%, including any amount in between and fractions thereof. In other embodiments, Form 1 has a purity level of between about 96.0% and 98%. In certain embodiments, Form 1 has a purity level of at least 96.0%.

In some embodiments, any one of Form 2. Form 3, Form 4, Form 5, Form 6, Form 7. Form 8, Form 9, Form 10, Form 11, Form 12, or Form 13 of Compound A may have a purity level of at least about 95.0%, about 96.0%, about 97.0%, about 98.0%, about 99.0%, about 99.1%, about 99.2%, about 99.3%, about 99.4%, about 99.5%, about 99.6%, about 99.7%, about 99.8%, or about 99.9%, including any amount in between and fractions thereof.

### IV. Methods of Synthesis

In general, the present invention relates to methods of preparing Compound A, or salts thereof, or solvates of the foregoing. In one variation, provided are methods of preparing a diacetate salt of Compound A. In one variation, provided are methods of preparing Form 1 of a Compound A diacetate salt.

In one aspect, provided herein is a method for preparing a Compound A diacetate salt, comprising reacting Compound A with an acetate salt in the presence of a solvent. In some embodiments, the solvent is a polar solvent. In some embodiments the solvent is an alcohol. In some embodiments, the solvent is methanol. In some embodiments, the acetate salt is ammonium acetate. In some embodiments the diacetate salt of Compound A is Form 1 of the Compound A diacetate salt. In some embodiments, the method comprises admixing Compound A, the acetate salt, and the solvent, and heating the mixture to about 30° C. for about 12 hours, then heating the mixture to about 35° C. for about 2 hours. In some embodiments, the method further comprising filtering the reaction mixture. In some embodiments, the filtering is performed under vacuum at about 40° C. In some embodiments, the method further comprises slurrying the reaction mixture with acetone at about 30° C. In some embodiments, the method further comprises slurrying the reaction mixture with n-heptane at between about 60° C. and 65° C. In some embodiments, the method further comprises drying the product under vacuum at between about 60° C. and 70° C. In some embodiments, the method further comprises preparing Compound A according to any of the methods described herein.

In some embodiments, provided is a method for preparing Compound A, comprising (i) reacting 5-((5-(4-cyanophenoxy)pentyl)oxy)picolinimidamide acetate (INT-3) with an acid in the presence of ethanol to provide ethyl 4-((5-((6-carbamimidoylpyridin-3-yl)oxy)pentyl)oxy)benzimidate (INT-4), and (ii) reacting ethyl 4-((5-((6-carbamimidoylpyridin-3-yl)oxy)pentyl)oxy)benzimidate (INT-4) with an ammonium source and a base, in the presence of a solvent, to provide Compound A.

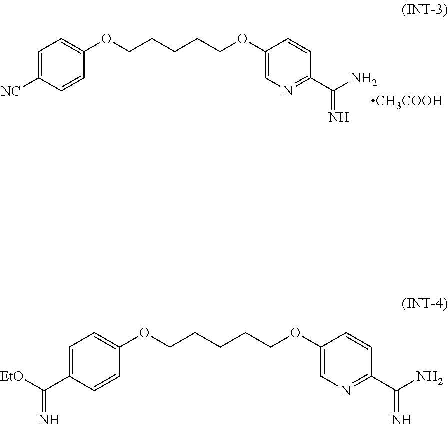

In some embodiments, the acid is hydrochloric acid. In some embodiments, the reaction of (INT-3), the acid, and ethanol, is performed at between about 15° C. to 20° C. In some embodiments, the method further comprises heating the reaction mixture to about 30° C. In some embodiments, the method further comprises holding the reaction mixture at about 30° C. for about 12 hours. In some embodiments, the reaction of (INT-4), the ammonium source, and the base is performed in the presence of methanol as a solvent. In some embodiments the ammonium source and the base are both ammonium carbonate. In some embodiments, the reaction of (INT-4), the ammonium source, and the base occurs at 25° C. In some embodiments, the reaction mixture is held at 25° C. for about 10 hours. In some embodiments, the method further comprises preparing INT-3 according to any of the methods described herein.

In some embodiments, provided is a method for preparing 5-((5-(4-cyanophenoxy)pentyl)oxy)picolinimidamide acetate (INT-3), comprising reacting 5-((5-(4-cyanophenoxy)pentyl)oxy)picolinonitrile (INT-2) with (i) an alkali metal methoxide, and (ii) ammonium acetate in the presence of a solvent.

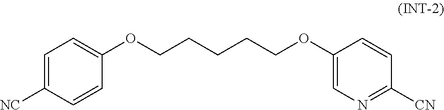 [SMILES: N#Cc1ccc(OCCCCCOc2ccc(C#N)nc2)cc1]

In some embodiments, the alkali metal methoxide is sodium methoxide. In some embodiments, the solvent is a polar solvent. In some embodiments, the solvent is an alcohol. In some embodiments, the solvent is methanol. In some embodiments, the method comprises admixing INT-2 with the alkali metal methoxide at about 10° C., and heating the reaction mixture to about 50° C. In some embodiments, the reaction mixture is held at about 50° C. for about 6 hours. In some embodiments, the ammonium acetate is added to the reaction mixture at about 30° C. In some embodiments, the reaction mixture is held at 30° C. for about 16 hours following addition of the ammonium acetate. In some embodiments, the method further comprises preparing INT-2 according to any of the methods described herein.

In some embodiments, provided is a method for preparing 5-((5-(4-cyanophenoxy)pentyl)oxy)picolinonitrile (INT-2), comprising reacting 4-((5-bromopentyl)oxy)benzonitrile (INT-1) with 5-hydroxypicolinonitrile (SM-3) and a base in the presence of a solvent.

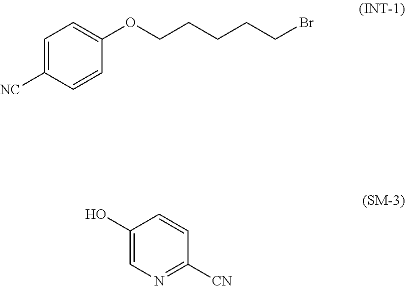 [SMILES: N#Cc1ccc(OCCCCCBr)cc1 ; N#Cc1ccc(O)cn1]

In some embodiments, the base is a carbonate salt. In some embodiments, the base is potassium carbonate. In some embodiments, the solvent is N,N-dimethylformamide (DMF). In some embodiments, the method comprises heating the reaction mixture to about 75° C. In some embodiments, the method comprises heating the reaction mixture to about 75° C. for about 1 hour. In some embodiments, the method further comprises cooling the reaction mixture to about 30° C. In some embodiments, the method further comprises quenching the reaction with water. In some embodiments, the method further comprises preparing INT-1 according to any of the methods described herein.

In some embodiments, provided is a method for preparing 4-((5-bromopentyl)oxy)benzonitrile (INT-1), comprising reacting 4-hydroxybenzonitrile (SM-1) with 1,5-dibromopentane (SM-2) and a base in the presence of a solvent.

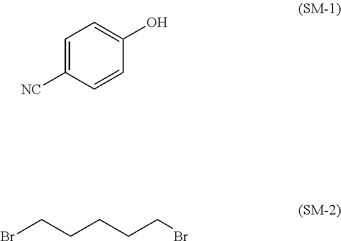 [SMILES: N#Cc1ccc(O)cc1 ; BrCCCCCBr]

In some embodiments, the solvent is N,N-dimethylformamide (DMF). In some embodiments, the base is a carbonate salt. In some embodiments, the base is potassium carbonate. In some embodiments, the method comprises admixing SM-2, the base, and a solution of SM-1 and the solvent. In some embodiments, the addition occurs over about one hour. In some embodiments, the reaction mixture is held at about 40° C. during the addition. In some embodiments, the method further comprises holding the reaction mixture at about 40° C. for about 4 hours following the addition. In some embodiments, the method further comprises cooling the reaction mixture to 30° C. In some embodiments, the method further comprises quenching the reaction mixture with water.

### V. Pharmaceutical Compositions

In general, the present invention relates to pharmaceutical forms and compositions of Compound A or pharmaceutically acceptable salts thereof, or solvates of the foregoing, corresponding pharmaceutical compositions, methods and/or uses for treatment of cancer as defined herein.

In one aspect, the present invention relates to a pharmaceutical composition of an acetate salt of Compound A:

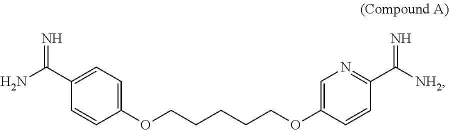 [SMILES: N=C(N)c1ccc(OCCCCCOc2ccc(C(=N)N)nc2)cc1]

or
a corresponding solvate thereof.

In another aspect, the acetate salt of Compound A or corresponding solvate thereof may be present in any form, such as a hydrate or other solvate.

In some aspects, the acetate salt of Compound A or solvate thereof may be provided in crystalline form, in an amorphous form, or a semi-crystalline form. In some aspects, the acetate salt of Compound A or solvate thereof is a crystalline form. In some aspects, the acetate salt of Compound A or solvate thereof is an amorphous form. In some aspects, the acetate salt of Compound A or solvate thereof is a semi-crystalline form. In one aspect, the composition of an acetate salt of Compound A or solvate thereof is a diacetate salt. In some aspects, the diacetate salt has from about 1.5 to about 2.5 molar equivalents of acetate compared to Compound A. In some aspects, the hemi acetate salt has about 1.5, 1.6, 1.7, 1.8, 1.9, 2.0, 2.1, 2.2, 2.3, 2.4, or about 2.5 molar equivalents of acetate compared to Compound A. In some aspects, the diacetate salt has about 2.0 molar equivalents of acetate compared to Compound A.

In some aspects, the acetate salt form of Compound A or solvate thereof may be a hydrate. In some aspects, the hydrate of the acetate salt of Compound A has from about 0.2 to about 10 molar equivalents of water compared to Compound A. In some embodiments, the hydrate of the acetate salt of Compound A has about 1 or about 4 molar equivalents of water compared to Compound A.

The acetate salt compositions of the present invention can be administered to a subject or patient by any means in accordance with therapeutic administration, which accomplishes intended purpose or pharmaceutical efficacy. Examples include administration by oral, parenteral, subcutaneous, intravenous, intramuscular, intraperitoneal, transdermal, topical, buccal or ocular routes. In some aspects, the administration of the acetate salt composition of the present invention is adapted for oral administration.

In another aspect, the present invention provides a composition which comprises an acetate salt of Compound A or solvate thereof in an amount of from about 0.1% to about 60% (w/w) of the composition and one or more pharmaceutically acceptable excipients.

In another aspect, the present invention provides a composition, which comprises: an acetate salt of Compound A or solvate thereof; and about 50 mM pH 7.4 phosphate buffered aqueous solution.

In another aspect, the present invention provides a composition which comprises an acetate salt of Compound A or solvate thereof, one or more fillers, a glidant, and a lubricant. In another aspect, the present invention provides a composition which comprises an acetate salt of Compound A or solvate thereof in an amount of from about 0.10% to about 60% (w/w) of the composition, one or more fillers in an amount of from about 10% to about 90% (w/w) of the composition, a glidant in an amount of from about 0.1% to about 5% (w/w) of the composition, and a lubricant in an amount of from about 0.1% to about 5% (w/w) of the composition.

In another aspect, the present invention provides a composition which comprises an acetate salt of Compound A or solvate thereof, silicified microcrystalline cellulose, lactose monohydrate, colloidal silicon dioxide, and magnesium stearate. In another aspect, the present invention provides a composition which comprises an acetate salt of Compound A or solvate thereof in an amount of from about 0.1% to about 60% (w/w) of the composition, silicified microcrystalline cellulose in an amount of from about 10% to about 70% (w/w) of the composition, lactose monohydrate in an amount of from about 5% to about 30% (w/w) of the composition, colloidal silicon dioxide in an amount of from about 0.1% to about 5% (w/w) of the composition, and magnesium stearate in an amount of from about 0.1% to about 5% (w/w) of the composition.

In another aspect, the acetate salt of Compound A or solvate thereof, may be present in any amount from about 0.1% to about 60% (w/w) of the composition. For example, the acetate salt of Compound A or solvate thereof, may be present in an amount of from about 15% to about 35% (w/w), or from about 40% to about 60%. In some variations, the acetate salt of Compound A may be present in an amount of about 25%. In some variations, the acetate salt of Compound A may be present in an amount of about 50%.

In some variations, provided is a composition comprising Form 1. Form 2, Form 3. Form 4, Form 5, Form 6, Form 7, Form 8, Form 9, Form 10, Form 11, or Form 13 of an acetate salt of Compound A in an amount of from about 0.1% to about 60% (w/w) of the composition. In some embodiments, the composition comprises Form 12 of a phosphate salt of Compound A in an amount of from about 0.1% to about 60% (w/w) of the composition.

In some embodiments, the composition comprises Form 1 of an acetate salt of Compound A in an amount of from about 0.1% to about 60% (w/w) of the composition. In some variations, Form 1 may be present in an amount of about 25%. In some variations, Form 1 may be present in an amount of about 50%.

In another aspect, the acetate salt of Compound A or solvate thereof may be present in any amount, such as an amount of from about 1 mg to about 1000 mg, or from about 1 mg to about 500 mg, from about 1 mg to about 100 mg, from about 10 mg to about 50 mg, from about 20 mg to about 40 mg, or from about 20 mg to about 30 mg. In another aspect, the amount of the acetate salt of Compound A or solvate thereof may be from about 1 mg to about 1000 mg. In another aspect, the amount of the acetate salt of Compound A or solvate thereof may be from about 5 mg to about 300 mg. In another aspect, the amount of the acetate salt of Compound A or solvate thereof is from about 25 mg to about 150 mg. In another aspect, the amount of the acetate salt of Compound A or solvate thereof may be from about 25 mg to about 100 mg. In another aspect, the acetate salt of Compound A or solvate thereof may be present in an amount of from about 1 mg to about 100 mg. In another aspect, the acetate salt of Compound A or solvate thereof may be present in an amount of from about 20 mg to about 40 mg. In another aspect, the acetate salt of Compound A or solvate thereof may be present in an amount of from about 20 mg to about 30 mg.

In yet another aspect, the acetate salt of Compound A or solvate thereof may be present in an amount of about 5 mg, about 10 mg, about 20 mg, about 30 mg, about 40 mg, about 50 mg, about 75 mg, about 100 mg, about 120 mg, or about 150 mg, including any amount in between and fractions thereof. In another aspect, an amount of the acetate salt of Compound A or solvate thereof may be present in about 5 mg. In another aspect, an amount of the acetate salt of Compound A or solvate thereof may be present in about 10 mg. In another aspect, an amount of the acetate salt of Compound A or solvate thereof may be present in about 20 mg. In another aspect, an amount of the acetate salt of Compound A or solvate thereof may be present in about 30 mg. In another aspect, an amount of the acetate salt of Compound A or solvate thereof may be present in about 40 mg. In another aspect, an amount of the acetate salt of Compound A or solvate thereof may be present in about 50 mg. In another aspect, an amount of the acetate salt of Compound A or solvate thereof may be present in about 75 mg. In another aspect, an amount of the acetate salt of Compound A or solvate thereof may be present in about 100 mg. In another aspect, an amount of the acetate salt of Compound A or solvate thereof may be present in about 120 mg. In another aspect, an amount of the acetate salt of Compound A or solvate thereof may be present in about 150 mg.

In another aspect, the amount of the crystalline form of a pharmaceutically acceptable salt of Compound A or solvate thereof may be present in an amount of about 5 mg, about 10 mg, about 20 mg, about 30 mg, about 40 mg, about 50 mg, about 75 mg, about 100 mg, about 120 mg, about 150 mg, about 200 mg, about 250 mg, about 300 mg, about 400 mg, about 500 mg, about 600 mg, about 700 mg, about 800 mg, about 900 mg, about 1000 mg, about 1100 mg, about 1200 mg, about 1300 mg, or about 1400 mg, including any amount in between and fractions thereof

In general, pharmaceutical compositions of the present invention may be formed into different dosage forms prepared using conventional materials and techniques known in the pharmaceutical and formulary arts, which may include, but is not limited to techniques, such as mixing, blending and the like and as set forth throughout the instant disclosure. Moreover, pharmaceutical composition used to form dosage forms may also include, but are not limited to, suitable adjuvants, carriers, excipients, or stabilizers, etc. and can be in solid or liquid form such as, solid or liquid dosage forms, which may include, but are not limited to tablets, capsules, powders, solutions, suspensions, or emulsions and the like, etc. In accordance with the present invention, solid unit dosage forms may be other conventional types known in the art.

Suitable compositions of the present invention may be in different forms, including, but are not limited to a liquid, a tablet, a capsule, etc. and the like. In some aspects, the composition may be a tablet composition or a capsule composition.

Further, suitable for use in the present invention are solutions, which may, but are not limited to, such as in water, saline, aqueous dextrose and related sugar solutions, and glycols such as, propylene glycol or polyethylene glycol, buffered solutions and the like, etc., are preferred liquid carriers, particularly for injectable solutions. Under ordinary conditions of storage and use, these preparations contain a preservative to prevent the growth of microorganisms.

The compositions of the present invention may include a variety of other pharmaceutically acceptable components or excipients, such as, including, but is not limited to, a glidant, a lubricant, a disintegrant, a binder, a desiccant, a filler, and other components or excipients and the like. These components are described within.

In accordance with the present invention, compositions as described herein may include at least one filler. In some aspects, a composition of the present invention may comprise a filler including, but is not limited to, one or more of alpha cellulose, beta cellulose, gamma cellulose, silicified microcrystalline cellulose, starch, modified-starch, sorbitol, mannitol, lactose, dextrose, sucrose, dibasic calcium phosphate, tribasic calcium phosphate, or calcium carbonate and the like. In some aspects, a composition of the present invention may include mannitol. In other aspects, a composition of the present invention may include sorbitol.

Representative fillers for use in the compositions of the present invention may include, but are not limited to, starch, lactitol, lactose, an inorganic calcium salt, microcrystalline cellulose, silicified microcrystalline cellulose sucrose, combinations thereof and the like. Additional fillers or diluents for use in the compositions of the present invention, may include, but are not limited to fillers or diluents conventionally known in the art, i.e., which are typically used in formulation of pharmaceutical compounds. Examples of such fillers or diluents for use in accordance with the present invention may include, but are not limited to sugars such as lactose, dextrose, glucose, sucrose, cellulose, starches and carbohydrate derivatives, polysaccharides (including dextrates and maltodextrin), polyols (including mannitol, xylitol, and sorbitol), cyclodextrins, calcium carbonates, magnesium carbonates, microcrystalline cellulose, combinations thereof, and the like. In some aspects, such fillers or diluents suitable for use in the present invention may include, but are not limited to lactose, microcrystalline cellulose, combinations thereof and the like.

Moreover, in another aspect, a filler for use in the present invention may be present in an amount of from about 1% to about 99% (w/w) of the composition, or from about 1% to about 60%, or from about 1% to about 25%, or from about 1% to about 20%, or from about 1% to about 10%, or from about 2% to about 8%, or from about 3% to about 5% (w/w) of a composition as defined in the instant specification. Moreover, such a filler may also be present in an amount of about 1%, 2%, 3%, 4%, 5%, 6%, 7%, 8%, 9%, or about 10% (w/w) of the composition, which may include any fractional amount in between those as defined.

In some embodiments, the filler is present in an amount from about 10% to about 60% (w/w) of the composition as defined in the instant specification. In some embodiments, the filler is present in an amount from about 40% to about 60% (w/w) of the composition as defined in the instant specification.

In some aspects, the composition further can include microcrystalline cellulose. Several types of microcrystalline cellulose may be suitable for use in compositions described herein, for example, microcrystalline cellulose may be selected from, but is not limited to MICROCEL® or AVICEL® types: PH101, PH102, PH103, PH105, PH 112, PH113, PH200, PH301, and the like and other types of microcrystalline cellulose, such as silicified microcrystalline cellulose. In one aspect, a composition for use in the present invention may include microcrystalline cellulose (AVICEL PH102). In another aspect, a composition suitable for use in the present invention may include microcrystalline cellulose (AVICEL PH101).

In another aspect, a microcrystalline cellulose may be present in an amount of from about 1% to about 99% (w/w) of the composition, or from about 1% to about 60%, or from about 1% to about 25%, or from about 1% to about 20%, or from about 1% to about 10%, or from about 2% to about 8%, or from about 3% to about 5% (w/w) of a composition as defined in the instant specification. In some aspects, a microcrystalline cellulose may also be present in an amount of about 1%. 2%, 3%, 4%, 5%, 6%, 7%, 8%, 9%, or about 10% (w/w) of the composition, which may include any fractional amount in between those as defined. In some aspects, a microcrystalline cellulose may also be present in an amount of from about 3% to about 5% (w/w) of a composition.

In some aspects, the composition further can include a silicified microcrystalline cellulose. In some aspects, silicified microcrystalline cellulose may be, but is not limited to SMCC 50, SMCC 50LD, SMCC 90, SMCC HD90 or SMCC 90LM and the like. In some aspects, silicified microcrystalline cellulose may be SMCC 50, SMCC 50LD, SMCC 90, SMCC HD90 or SMCC 90LM. Without being bound by theory, the silicified microcrystalline cellulose is understood to protect an enteric coating from premature erosion by sodium caprate present in the composition. The silicified microcrystalline cellulose may be present in any suitable amount for use in the present invention. For example, the SMCC can be present in an amount of from about 1% to about 99% (w/w) of the composition, or from about 10% to about 60%, or from about 20% to about 50%, or from about 25% to about 45%, or from about 30% to about 40%, or from about 35% to about 37% (w/w) of the composition. In some aspects, the amount of the silicified microcrystalline cellulose is from about 30% to about 70% (w/w) of the composition. In some aspects, the amount of the silicified microcrystalline cellulose is from about 65% to about 85% (w/w) of the composition. In some aspects, the amount of the silicified microcrystalline cellulose is from about 66.5% to about 81.3% (w/w) of the composition. In some aspects, the amount of the silicified microcrystalline cellulose is about 31.3%, about 36.6%, about 37.7%, about 50.9%, about 52%, about 65.2%, about 71.5%, about 79%, or about 80.5% of the composition. The SMCC can be present in an amount of about 30% (w/w) of the composition, or about 31%, 32%, 33%, 34%, 35%, 36%, 36.1%, 36.2%, 36.3%, 36.4%, 36.5%, 36.6%, 36.7%, 36.8%, 36.9%, 37%, 38%, 39%, or about 40% (w/w) of the composition.

In some embodiments, SMCC is present in an amount of from about 20% to about 90% (w/w), which includes, but is not limited to any fractional amount in between. In some embodiments, SMCC is present in an amount of from about 25% to about 85% (w/w), which includes, but is not limited to any fractional amount in between. In some embodiments, SMCC is present in an amount of from about 25% to about 45% (w/w), which includes, but is not limited to any fractional amount in between. In some embodiments, SMCC is present in an amount of from about 30% to about 40% (w/w), which includes, but is not limited to any fractional amount in between. In some embodiments, SMCC is present in an amount of from about 65% to about 90% (w/w), which includes, but is not limited to any fractional amount in between. In some embodiments, SMCC is present in an amount of from about 70% to about 85% (w/w), which includes, but is not limited to any fractional amount in between. In some embodiments, SMCC is present in an amount of from about 70% to about 75% (w/w), which includes, but is not limited to any fractional amount in between. In some embodiments, SMCC is present in an amount of from about 80% to about 85% (w/w), which includes, but is not limited to any fractional amount in between. In some embodiments, SMCC is present in an amount of about 30%. In some embodiments. SMCC is present in an amount of about 40%, which includes, but is not limited to any fractional amount in between. In some embodiments. SMCC is present in an amount of about 50%, which includes, but is not limited to any fractional amount in between. In some embodiments, SMCC is present in an amount of about 60%, which includes, but is not limited to any fractional amount in between. In some embodiments, SMCC is present in an amount of about 70%, which includes, but is not limited to any fractional amount in between. In some embodiments, SMCC is present in an amount of about 80%, which includes, but is not limited to any fractional amount in between. In some embodiments, SMCC is present in an amount of about 90%, which includes, but is not limited to any fractional amount in between.

In some embodiments. SMCC is a mixture of microcrystalline cellulose and colloidal silicon dioxide.

In some aspects, the composition further can include one or more of alpha cellulose, beta cellulose, gamma cellulose, starch, modified-starch, sorbitol, mannitol, lactose, dextrose, sucrose, dibasic calcium phosphate, tribasic calcium phosphate, or calcium carbonate. In some aspects, the composition further can include mannitol.

In some aspects, a composition of the present invention may include lactose. In some embodiments, the lactose is lactose monohydrate. For example, for use in the present invention, lactose may be present in an amount of from about 1% to about 99% (w/w) of the composition, or from about 1% to about 50%, or from about 1% to about 25%, or from about 5% to about 25%, or from about 5% to about 20%, or from about 5 to about 15%, or from about 8 to about 12% (w/w) of the composition. In another aspect, lactose can be present in an amount of about 15% (w/w) of the composition. In another aspect, lactose can be present in an amount of about 25% (w/w) of the composition.

The composition of the invention may include, but is not limited to at least one disintegrant in an effective therapeutic amount for use as determined in accordance with the present invention. Representative disintegrants for use in the present invention, include, but are not limited to, starches, clays, celluloses, alginates and gums and crosslinked starches, celluloses and polymers, combinations thereof and the like. Additional representative disintegrants for use in the present invention, may include, but are not limited to microcrystalline cellulose, croscarmellose sodium, alginic acid, sodium alginate, crospovidone, cellulose, agar and related gums, sodium starch glycolate, corn starch, potato starch, sodium starch glycolate, Veegum HV, methylcellulose, agar, bentonite, carboxymethylcellulose, alginic acid, guar gum combinations thereof, and the like.

In another aspect, a composition of the present invention may also include, but is not limited to silica in any amount for purposes of the present invention. In particular, silica is exemplified by Aerosil 200, having a specific surface area of about 200 m²/g. Alternatives to silica may include, but are not limited to talc, sodium ferrocyanide, potassium ferrocyanide, calcium carbonate, magnesium carbonate, silicon dioxide, precipitated silica, sodium aluminosilicate, combinations thereof and the like.

In some aspects, a composition of the present invention may further comprise a silica. In one aspect, silica may be present in compositions of the present invention in an amount of from about 0.10% to about 10% (w/w) of the composition, or from about 0.10% to about 5%, or from about 0.1% to about 2%, or from about 0.1% to about 1.5%, or from about 0.1% to about 1%, or from about 0.3% to about 0.7% (w/w) of the composition of the present invention. For example, silica as used in the present invention may be present in an amount of about 0.1%, 0.2%, 0.3%, 0.4%, 0.5%, 0.6%, 0.7%, 0.8%, 0.9%, 1.0%, or about 1.5% (w/w) of the composition, including any fraction amount in between as defined. In another aspect, a composition of the present invention may further include an amount of silica in from about 0.1% to about 1.5% (w/w) of the composition. In another aspect, a composition of the present invention may further include an amount of silica in from about 0.5% to about 2% (w/w) of the composition. In another aspect, a composition of the present invention may further include an amount of silica in from about 0.3% to about 0.7% (w/w) of the composition. In further aspects, a composition of the present invention may further include an amount of silica in about 0.5% (w/w) of the composition. In some aspects, the composition further may comprise silica in an amount of about 1% (w/w) of the composition. Examples of suitable silica materials include, but are not limited to colloidal silicon dioxide, aerosol, colloidal silica, fumed silica, silicon dioxide fumed, colloidal anhydrous silica, colloidal silicon dioxide, and the like. In some embodiments, the silica is colloidal silica.

The composition can also include a binder. Binders for use in the compositions of the present invention include binders commonly used in the formulation of pharmaceuticals. Examples of binders for use in the present invention include but are not limited to cellulose derivatives (including hydroxypropyl cellulose, hydroxypropyl methylcellulose, methylcellulose, and sodium carboxymethyl cellulose), glycol, sucrose, dextrose, corn syrup, polysaccharides (including acacia, targacanth, guar, alginates and starch), corn starch, pregelatinized starch, modified corn starch, gelatin, polyvinylpyrrolidone, polyethylene, polyethylene glycol, combinations thereof and the like.

In the present invention, the composition may include a lubricant in any suitable amount for use as described herein. Examples of suitable lubricants for use in the present invention, may include, but are not limited to magnesium carbonate, magnesium lauryl sulphate, calcium silicate, talc, fumed silicon dioxide, combinations thereof, and the like. Other useful suitable lubricants, may include, but are not limited to magnesium stearate, calcium stearate, stearic acid, sodium stearyl fumarate, polyethylene glycol, sodium lauryl sulphate, magnesium lauryl sulphate, sodium benzoate, colloidal silicon dioxide, magnesium oxide, microcrystalline cellulose, starches, mineral oil, waxes, glyceryl behenate, polyethylene glycol, sodium acetate, sodium chloride, combinations thereof, and the like.

In some aspects, lubricant may include, but is not limited to magnesium stearate. In one aspect, an amount of the lubricant can be present in from about 1% to about 10% (w/w) of the composition, or from about 0.1% to about 5%, or from about 0.1% to about 1%, or from about 0.1% to about 0.5% (w/w) of the composition. In some aspects, an amount of the lubricant can be present in from about 0.1% to about 0.5% (w/w) of the composition. In some aspects, the amount of the lubricant is from about 0.3% to about 0.7% (w/w) of the composition. In some aspects, the amount of the lubricant is about 0.5% (w/w) of the composition. The lubricant can also be present in an amount of about 0.10% (w/w) of the composition, or about 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, or about 0.30% (w/w) of the composition. In some aspects, the lubricant may be present in an amount of about 0.25% (w/w).

In some aspects, the composition includes: (i) the acetate salt of Compound A or solvate thereof in an amount of about 27% (w/w) of the composition; (ii) a silicified microcrystalline cellulose in an amount of about 47.25% (w/w) of the composition; (iii) lactose monohydrate in an amount of about 23.6% (w/w) of the composition; (iv) a silica in an amount of about 1.6% (w/w) of the composition; and (v) magnesium stearate in an amount of about 0.5% (w/w) of the composition. In some embodiments, the composition is encapsulated in a capsule. In some embodiments, the capsule is a HPMC capsule.

In some aspects, the composition includes: (i) the acetate salt of Compound A or solvate thereof in an amount of about 50.8% (w/w) of the composition; (ii) a silicified microcrystalline cellulose in an amount of about 31.4% (w/w) of the composition; (iii) lactose monohydrate in an amount of about 15.7% (w/w) of the composition; (iv) a silica in an amount of about 1.6% (w/w) of the composition; and (v) magnesium stearate in an amount of about 0.5% (w/w) of the composition. In some embodiments, the composition is encapsulated in a capsule. In some embodiments, the capsule is a HPMC capsule.

The composition described herein may include a variety of other pharmaceutically acceptable components or excipients, such as, including, but is not limited to, a glidant, a lubricant, a disintegrant, a binder, a desiccant, a filler, and other components or excipients and the like.

The composition described herein can include at least one disintegrant in any suitable amount in accordance with the present invention. Representative disintegrants for use in the present invention, may include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums (like gellan), low-substituted hydroxypropyl cellulose, or mixtures thereof and the like. In one aspect, the disintegrant may include croscarmellose sodium. In one aspect, the disintegrant may include crospovidone. In another aspect, suitable disintegrant may be, but is not limited to being present in an amount of about 1% (w/w). 2%, 3%, 4%. 5%, 6%, 7%, 8%, 9%, or about 10% (w/w) of the composition, including any fractional amount in between as defined in the present invention. In another aspects of the present invention, disintegrant may be, but is not limited to being present in an amount of about 1 to 10% (w/w) of the composition. In other aspects, the disintegrant may be present in an amount of about 5.0% (w/w) of the composition.

In some aspects, the composition further may comprise silica. In some aspects, the composition further may comprise silica in an amount of from about 0.1% to about 2% (w/w) of the composition. For example, the silica can be present in an amount of about 0.10%, 0.2%, 0.25%, 0.3%, 0.4%, 0.5%, 0.6%, 0.7%, 0.75%, 0.8%, 0.9%, 1.0%, 1.1%, 1.2%, 1.3%, 1.4%, 1.5%, 1.6%, 1.7%, 1.8%, 1.9% or about 2% (w/w) of the composition, including any fraction amount in between as defined herein. In some aspects, the composition further may comprise silica in an amount of from about 0.3% to about 0.7% (w/w) of the composition. In some aspects, the composition further may comprise silica in an amount of from about 0.5% to about 1.6% (w/w) of the composition. In some aspects, the composition further may comprise silica in an amount of about 0.5% (w/w) of the composition.

The microcrystalline cellulose can include any microcrystalline cellulose known in the art. In some aspects, the microcrystalline cellulose may comprise a silicified microcrystalline cellulose (SMCC).

In some aspects, for use in the present invention, microcrystalline cellulose may be a silicified microcrystalline cellulose (SMCC) and may have any particle size.

The composition can include at least one disintegrant in any suitable amount in accordance with the present invention. Representative disintegrants for use in the present invention, may include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums (like gellan), low-substituted hydroxypropyl cellulose, or mixtures thereof and the like. In one aspect, the disintegrant may include croscarmellose sodium. In one aspect, the disintegrant may include crospovidone. The disintegrant for use in the present invention, may be, but is not limited to being present in an amount of from about 1% to about 99% (w/w) of the composition, or from about 1% to about 50%, or from about 1% to about 25%, or from about 1% to 20%, or from about 1% to about 10%, or from about 2% to about 8%, or from about 4% to about 6% (w/w) of the composition. In another aspect, suitable disintegrant may be, but is not limited to being present in an amount of about 1% (w/w), 2%, 3%. 4%, 5%, 6%, 7%, 8%. 9%, or about 10% (w/w) of the composition, including any fractional amount in between as defined in the present invention. In another aspects of the present invention, disintegrant may be, but is not limited to being present in an amount of about 1% to about 10% (w/w) of the composition. In other aspects, the disintegrant may be present in an amount of about 5.0% (w/w) of the composition.

In another aspect, the composition may also include silica in any amount in accordance with the present invention. Silica is exemplified by Aerosil 200, having a specific surface area of about 200 m²/g. Alternatives to silica include, without limitation, talc, sodium ferrocyanide, potassium ferrocyanide, calcium carbonate, magnesium carbonate, silicon dioxide, precipitated silica, sodium aluminosilicate, and combinations thereof and the like. Silica (e.g., Aerosil 200) may be present in the compositions in an amount of from about 0.1 to 10% (w/w) of the composition, or from about 0.1 to 5%, or from about 0.1 to 2%, or from about 0.1 to 1.5%, or from about 0.1 to 1%, or from about 0.3 to 0.7% (w/w) of the composition. For example, the Aerosil 200 silica can be present in an amount of about 0.1%, 0.2%, 0.3%, 0.4%, 0.5%, 0.6%, 0.7%. 0.8%, 0.9%, 1.0%, or 1.5% (w/w) of the composition, including any fraction amount in between.

In some aspects, the composition further may comprise silica (e.g., Aerosil 200). In some aspects, the composition further may comprise silica (e.g., Aerosil 200) in in an amount of from about 0.1% to about 1.5% (w/w) of the composition. For example, the silica can be present in an amount of about 0.1%, 0.2%, 0.25%, 0.3%, 0.4%, 0.5%, 0.6%, 0.7%, 0.75%, 0.8%, 0.9%, 1.0%, or about 1.5% (w/w) of the composition, including any fraction amount in between as defined herein. In some aspects, the composition further may comprise silica (e.g., Aerosil 200) in an amount of from about 0.3% to about 0.7% (w/w) of the composition. In some aspects, the composition further may comprise silica (e.g., Aerosil 200) in an amount of from about 0.5% to about 2% (w/w) of the composition. In some aspects, the composition further may comprise silica in an amount of about 0.5% (w/w) of the composition. In some aspects, the composition further may comprise silica (e.g., Aerosil 200) in an amount of about 1% (w/w) of the composition.

The composition described herein can include a variety of other pharmaceutically excipients or components, which may include, but is not limited to a lubricant, a disintegrant, a binder, a desiccant, a filler, and other components and the like. For use in the present invention, a disintegrant may be present in the compositions in an amount of from about 0.1% to about 10% (w/w) of the composition, or from about 0.1% to about 5%, or from about 0.1% to about 2%, or from about 0.1% to about 1.5%, or from about 0.10% to about 1%, or from about 0.10% to about 0.4% (w/w) of the composition. In some aspects, the composition further may comprise a disintegrant. In some aspects, the composition further may comprise silica (e.g., Aerosil 200) in an amount of from about 0.1% to about 1.5% (w/w) of the composition. In some aspects, the composition further may comprise a disintegrant in an amount of about 0.25% (w/w) of the composition.

In some aspects, the compositions disclosed herein can further comprise at least one of: a lubricant in an amount from about 0.10% to about 0.5% by weight of the composition, a disintegrant in an amount from about 1% to about 10% by weight of the composition, or a silica (e.g., Aerosil 200) in an amount from about 0.1% to about 1.5% by weight of the composition.

In some aspects, the compositions further can include: a lubricant in an amount from about 0.1% to about 0.5% by weight of the composition; a disintegrant in an amount from about 1% to about 10% by weight of the composition; and a silica (e.g., Aerosil 200) in an amount from about 0.1% to about 1.5% by weight of the composition.

In some aspects, a composition of the present invention may be in a dosage form, which may be, but is not limited to a tablet or capsule dosage form. In some aspects, the composition may be a tablet or capsule composition. In some aspects, the composition can be a tablet composition. In some aspects, the composition can be a capsule composition. In some aspects, the composition may comprise a unit dose size in amounts which may include, but is not limited to amounts from about 20 mg to about 2000 mg, from about 500 mg to about 2000 mg. Compositions of the present invention, may be of any suitable size in accordance with the present invention, such as, but not limited tablets or capsules in doses or amounts of 20, 30, 40, 50, 75, 100, 110, 120, 130, 140, 150, 160, 170, 180, 190, 200, 250, 300, 350, 400, 450, 500, 550, 600, 650, 700, 750, 800, 850, 900, 950, 1050, 1100, 1150, 1200, 1250, 1300, 1350, 1400, 1450, 1500, 1550, 1600, 1650, 1700, 1750, 1800, 1850, 1900, 1950 or 2000 milligrams (mg) and the like. In one aspect, a composition of the present invention may be as a 20 mg, 30 mg, 40 mg, 50 mg, 100 mg, 120 mg, 150 mg, 200 mg, 250 mg, 300 mg, 500 mg, 600 mg, 800 mg, 1000 mg, 1200 mg, or 1400 mg tablet, respectively, which may be administered, but is not limited to once or twice daily or as determined by medical necessity. In some aspects, the composition may be a unit dose size of from about 500 mg to about 2000 mg. In some aspects, the composition may be a unit dose size of about 1400 mg. In some aspects, the composition may be a unit dose size of about 1000 mg.

In some aspects, the composition may comprise a unit dose size from 500 mg to about 2000 mg. The tablet compositions may be of any suitable size in accordance with the present invention, such as, but not limited to 20, 30, 40, 50, 75, 100, 110, 120, 130, 140, 150, 160, 170, 180, 190, 200, 250, 300, 350, 400, 450, 500, 550, 600, 650, 700, 750, 800, 850, 900, 950, 1050, 1100, 1150, 1200, 1250, 1300, 1350, 1400, 1450, 1500, 1550, 1600, 1650, 1700, 1750, 1800, 1850, 1900, 1950 or 2000 mg tablets. In some aspects, the composition is a 1400 mg tablet.

Tablets or capsules formed from compositions of the present invention may be administered in single or multiple administrations depending on dosing and frequency as required and tolerated by the patient, where such tablets or capsules contain a sufficient quantity or amount of active agent to effectively treat specific disease state. Thus, in one aspect, the present invention relates to a composition for oral administration of the acetate salt of Compound A or solvate thereof, which may be taken in a daily amount of from about 0.05 to about 30 mg per kg of body weight per day. In some aspects, dosages can be from about 0.1 mg to about 20 mg per kg of body weight per day. In another aspect, dosages can be from about 0.1 mg to about 5 mg per kg of body weight per day. In another aspect, dosages can be from about 0.1 mg to about 1 mg per kg of body weight per day.

In any of the foregoing embodiments, the acetate salt of Compound A or solvate thereof may be Form 1.

### Coatings

### Cosmetic Subcoating

In some embodiments, the composition can further include a subcoating. In some embodiments, the subcoating is a cosmetic subcoating. In some embodiments, the cosmetic subcoating can also serve as a physical barrier. Cosmetic coatings can include polyethylene glycol-polyvinyl alcohol (PEG-PVA) graft copolymer, polyvinyl alcohol (PVA), hypromellose (HPMC), and hydroxypropyl cellulose (HPC). In some embodiments, the weight of the cosmetic subcoating is compared weight/weight to the weight of the composition prior to coating. In some embodiments, the cosmetic subcoating can be present in an amount from about 1% to about 10% (w/w). In some embodiments, the cosmetic subcoating is present in an amount from about 1% to about 5% (w/w). For example, the cosmetic subcoating can be present in amounts including about 1%, 1.5%, 2.0%, 2.5%, and about 3%, including any fractional amounts in between and ranges thereof, such as between about 2.0% and 3.0%. In some embodiments, the cosmetic subcoating is present in an amount of about 3% (w/w). In some embodiments, the weight of the cosmetic subcoating is compared weight/weight to the weight of the composition, or to the weight of the core tablet, prior to coating.

In other embodiments, the cosmetic subcoating level is indicated in terms of weight (mg) of the cosmetic subcoating per the surface area of the core tablet. In some embodiments, the surface area is the surface area of the outer most layer of a coating covering the core tablet. For example, in some embodiments, the surface area for calculating the cosmetic subcoating level is the surface area of the core tablet. In other embodiments, the surface area is the surface area of the cosmetic subcoating, subcoating, or enteric coating disposed over the core tablet. For example, in some embodiments, more than one coating is disposed over the core tablet and the surface area refers to the surface area of the outermost coating.

In some embodiments, the cosmetic subcoating level is from about 6 mg/cm² to about 30 mg/cm². In some embodiments, the cosmetic subcoating level is from about 9 mg/cm² to about 30 mg/cm². In other embodiments, the cosmetic subcoating level is from about 12 mg/cm² to about 30 mg/cm². In some embodiments, the cosmetic subcoating level is from about 17 mg/cm² to about 30 mg/cm². In some embodiments, the cosmetic subcoating level is from about 20 mg/cm² to about 30 mg/cm². In some embodiments, the cosmetic subcoating level is from about 25 mg/cm² to about 30 mg/cm². For example, the cosmetic subcoating level is about 6 mg/cm², 7 mg/cm², 8 mg/cm², 9 mg/cm², 10 mg/cm², 6 mg/cm², 11 mg/cm², 12 mg/cm², 13 mg/cm², 14 mg/cm², 15 mg/cm², 16 mg/cm², 17 mg/cm², 18 mg/cm², 19 mg/cm², 21 mg/cm², 22 mg/cm², 23 mg/cm², 24 mg/cm². 25 mg/cm², 26 mg/cm², 27 mg/cm², 28 mg/cm², 29 mg/cm², or 30 mg/cm². In some embodiments, the cosmetic subcoating level is about 6 mg/cm². In some embodiments, the cosmetic subcoating level is about 7 mg/cm². In some embodiments, the cosmetic subcoating level is about 8 mg/cm². In some embodiments, the cosmetic subcoating level is about 9 mg/cm². In some embodiments, the cosmetic subcoating level is about 10 mg/cm². In some embodiments, the cosmetic subcoating level is about 11 mg/cm². In some embodiments, the cosmetic subcoating level is about 6 mg/cm². In some embodiments, the cosmetic subcoating level is about 12 mg/cm². In some embodiments, the cosmetic subcoating level is about 13 mg/cm². In some embodiments, the cosmetic subcoating level is about 14 mg/cm². In some embodiments, the cosmetic subcoating level is about 15 mg/cm². In some embodiments, the cosmetic subcoating level is about 16 mg/cm². In some embodiments, the cosmetic subcoating level is about 17 mg/cm². In some embodiments, the cosmetic subcoating level is about 18 mg/cm².

### Subcoating

In some aspects, the composition further can include a subcoating of a PVA-PEG graft co-polymer disposed over the composition. In some aspects, compositions can comprise a subcoating of a PVA-PEG graft co-polymer disposed over the core tablet. This coating can serve as a smooth surface to aid in swallowing the tablet. It can also provide a platform for a further layer which can comprise an enteric coating disposed over the subcoating. In some aspects, the subcoating can also provide a vehicle for pigmentation for tablet identification. Other coatings include, without limitation, HPMC, HPC, PVA, Eudragit E based coatings and the like. In some aspects, the composition further can include a subcoating. This coating can serve as a barrier between the components of the core tablet and the enteric coating or functional coating. Subcoatings can include the OPADRY® class of products and can be present in any desired amounts. In some aspects, the weight of the subcoating is compared weight/weight to the weight of the composition prior to coating. In some aspects, the subcoating can be present in an amount from about 1% to about 10% (w/w). In some aspects, the subcoating is present in an amount from about 1% to about 5% (w/w). In some aspects, the subcoating can be present in an amount from about 1% to about 3% (w/w) relative to the core tablet prior to coating. For example, the subcoating can be present in amounts including about 1%, 1.5%, 2.0%, 2.5%, and about 3%, including any fractional amounts in between. In some aspects, the subcoating is present in an amount of about 3% (w/w). In some aspects, the weight of the subcoating is compared weight/weight to the weight of the composition, or to the weight of the core tablet, prior to coating.

In other embodiments, the subcoating level is measured in a coating weight gain (mg/cm²). In some embodiments, the subcoating level is from about 6 mg/cm² to about 30 mg/cm². In some embodiments, the subcoating level is from about 9 mg/cm² to about 30 mg/cm². In other embodiments, the subcoating level is from about 12 mg/cm² to about 30 mg/cm². In some embodiments, the subcoating level is from about 17 mg/cm² to about 30 mg/cm². In some embodiments, the subcoating level is from about 20 mg/cm² to about 30 mg/cm². In some embodiments, the subcoating level is from about 25 mg/cm² to about 30 mg/cm². For example, the subcoating level is about 6 mg/cm², 7 mg/cm², 8 mg/cm², 9 mg/cm², 10 mg/cm², 6 mg/cm², 11 mg/cm², 12 mg/cm², 13 mg/cm², 14 mg/cm², 15 mg/cm², 16 mg/cm², 17 mg/cm², 18 mg/cm², 19 mg/cm², 21 mg/cm², 22 mg/cm², 23 mg/cm², 24 mg/cm², 25 mg/cm². 26 mg/cm², 27 mg/cm², 28 mg/cm², 29 mg/cm², or 30 mg/cm². In some embodiments, the subcoating level is about 6 mg/cm². In some embodiments, the subcoating level is about 7 mg/cm². In some embodiments, the subcoating level is about 8 mg/cm². In some embodiments, the subcoating level is about 9 mg/cm². In some embodiments, the subcoating level is about 10 mg/cm². In some embodiments, the subcoating level is about 11 mg/cm². In some embodiments, the subcoating level is about 6 mg/cm². In some embodiments, the subcoating level is about 12 mg/cm². In some embodiments, the subcoating level is about 13 mg/cm². In some embodiments, the subcoating level is about 14 mg/cm². In some embodiments, the subcoating level is about 15 mg/cm². In some embodiments, the subcoating level is about 16 mg/cm². In some embodiments, the subcoating level is about 17 mg/cm². In some embodiments, the subcoating level is about 18 mg/cm².

### Enteric Coating

In some aspects, the composition includes an enteric coating disposed over the subcoating. In some aspects, the enteric coating is selected to provide release of the tablet contents at a pH range from about 5 to about 8. In some aspects, the enteric coating is a pH 5.5 enteric coating. Enteric coating can include, without limitation, those based on cellulose acetate phthalate (CAP), poly(methacrylic acid-co-methyl methacrylate), cellulose acetate trimellitate (CAT), poly(vinyl acetate phthalate) (PVAP) or hydroxypropyl methylcellulose phthalate (HPMCP). In some aspects, the enteric coating can be a methacrylic acid co-polymer.

In some embodiments, the enteric coating can include, without limitation, poly(methacrylic acid ethyl acrylate) (L100D-55), a combination methyl acrylate, methyl methacrylate and methacrylic acid (FS30D), hydroxypropyl methylcellulose acetate succinate (HPMC-AS), Type L HPMC-AS, or a co-polymer of ethyl methacrylate acrylate (e.g., Acryl-eze®).

In some aspects, the weight of the enteric coating is compared weight/weight to the weight of the composition prior to coating. In some aspects, the enteric coating can be present in an amount from about 1% to about 15% (w/w). In some aspects, the enteric coating can be present in an amount from about 2% to about 15% (w/w). In some aspects, the enteric coating can make up from about 5% to about 15% (w/w) relative to the core tablet of the compositions. For example, the amounts of enteric coating can be in an amount of about 5%, 6%, 7%, 8%, 9%, 10%, 11%, 12%, 13%, 14%, or about 15% (w/w), including fractions thereof. In some aspects, the enteric coating can be present in an amount of about 6% (w/w). In some aspects, the enteric coating can be present in an amount of about 7% (w/w). In some aspects, the enteric coating can be present in an amount of about 8% (w/w). In some aspects, the weight of the enteric coating is compared weight/weight to the weight of the core tablet prior to coating.

In other embodiments, the enteric coating level is measured in a coating weight gain (mg/cm²). In some embodiments, the enteric coating level is from about 6 mg/cm² to about 30 mg/cm². In some embodiments, the enteric coating level is from about 9 mg/cm² to about 30 mg/cm². In other embodiments, the enteric coating level is from about 12 mg/cm² to about 30 mg/cm². In some embodiments, the enteric coating level is from about 17 mg/cm² to about 30 mg/cm². In some embodiments, the enteric coating level is from about 20 mg/cm² to about 30 mg/cm². In some embodiments, the enteric coating level is from about 25 mg/cm² to about 30 mg/cm². For example, the enteric coating level is about 6 mg/cm², 7 mg/cm², 8 mg/cm², 9 mg/cm², 10 mg/cm², 6 mg/cm², 11 mg/cm², 12 mg/cm², 13 mg/cm², 14 mg/cm², 15 mg/cm², 16 mg/cm², 17 mg/cm², 18 mg/cm². 19 mg/cm², 21 mg/cm², 22 mg/cm². 23 mg/cm², 24 mg/cm², 25 mg/cm², 26 mg/cm², 27 mg/cm², 28 mg/cm², 29 mg/cm², or 30 mg/cm². In some embodiments, the enteric coating level is about 6 mg/cm². In some embodiments, the enteric coating level is about 7 mg/cm². In some embodiments, the enteric coating level is about 8 mg/cm². In some embodiments, the enteric coating level is about 9 mg/cm². In some embodiments, the enteric coating level is about 10 mg/cm². In some embodiments, the enteric coating level is about 11 mg/cm². In some embodiments, the enteric coating level is about 6 mg/cm². In some embodiments, the enteric coating level is about 12 mg/cm². In some embodiments, the enteric coating level is about 13 mg/cm². In some embodiments, the enteric coating level is about 14 mg/cm². In some embodiments, the enteric coating level is about 15 mg/cm². In some embodiments, the enteric coating level is about 16 mg/cm². In some embodiments, the enteric coating level is about 17 mg/cm². In some embodiments, the enteric coating level is about 18 mg/cm².

In some aspects, the tablet compositions of the present invention may have a subcoating of OPADRY® QX yellow in an amount of about 3% (w/w) and an enteric coating of Acryl-ezeC® yellow of about 6% (w/w).

In some aspects, the tablet compositions of the present invention may have a subcoating of OPADRY® QX yellow in an amount of about 3% (w/w) and an enteric coating of Acryl-eze® yellow of about 7% (w/w).

In some aspects, the tablet compositions of the present invention may have a subcoating of OPADRY® QX yellow in an amount of about 3% (w/w) and an enteric coating of Acryl-eze® yellow of about 8% (w/w).

### Order of Coatings

In some embodiments, the core tablet is covered by one or more of a cosmetic coating, a subcoating, an enteric coating, or any combination thereof. When more than one cosmetic coating, subcoating, and/or enteric coating covers the core tablet, such coatings may be applied in any order such that any of the cosmetic coating, subcoating, and/or enteric coating may be directly applied to the surface of the core tablet. In such instances, any additional cosmetic coating, subcoating, and/or enteric coating may be applied in any order subsequently.

In some embodiments, the core tablet is covered by a cosmetic subcoating. In some embodiments, the core tablet is covered by a cosmetic subcoating and then covered by an enteric coating. In some embodiments, the core tablet is covered by a cosmetic subcoating and then covered by a subcoating, In certain embodiments, the core tablet is covered by a cosmetic subcoating followed by a subcoating, and then followed by an enteric coating. In certain embodiments, the core tablet is covered by a cosmetic subcoating followed by an enteric coating, and then followed by a subcoating.

In other embodiments, the core tablet is covered by a subcoating. In some embodiments, the core tablet is covered by a subcoating and then covered by an enteric coating. In some embodiments, the core tablet is covered by a subcoating and then covered by a cosmetic subcoating. In certain embodiments, the core tablet is covered by a subcoating followed by an enteric coating and then covered by a cosmetic subcoating. In certain embodiments, the core tablet is covered by a subcoating followed by a cosmetic subcoating and then covered by an enteric coating.

In other embodiments, the core tablet is covered by an enteric coating. In other embodiments, the core tablet is covered by an enteric coating and then covered by a subcoating. In certain embodiments, the core tablet is covered by an enteric coating and then covered by a cosmetic subcoating. In other embodiments, the core tablet is covered by an enteric coating followed by a subcoating and then covered by a cosmetic coating. In certain embodiments, the core tablet is covered by an enteric coating followed by a cosmetic coating and then covered by a subcoating.

In some aspects, the tablet compositions of the present invention may have a bioavailability of from about 1% to about 10% (w/w). In some aspects, the tablet compositions of the present invention may have a bioavailability of from about 10% to about 50%. For example, bioavailability may be about 1%, 2%, 3%, 4%, 5%, 6%, 7%, 8%, 9%, or about 10%. Bioavailability can be measured using area under curve (AUC) for oral dosing versus AUC by intravenous dosing.

A dose of a composition described herein may be administered according to a method and/or use of the present invention herein. In some aspects, a dose of a composition of the present invention can be administered once daily, twice daily, or three times daily. In some aspects, a dose of a composition of the present invention can be administered once daily. In some aspects, a dose of a composition of the present invention can be administered twice daily. In some aspects, a dose of a composition of the present invention can be administered three times daily.

In some embodiments described herein, such as relating to pharmaceutical compositions, tablets, methods, processes, and the like, the acetate salt of Compound A is excluded. In some embodiments described herein, such as relating to compositions, tablets, methods, processes, and the like, the acetate salt of Compound A is included.

### VI. Methods or Processes of Making Dosage Forms

In accordance with the present invention, pharmaceutical compositions are comprised of active principal ingredient (i.e., an acetate salt of Compound A or solvate thereof) and at least one or more additional pharmaceutically acceptable ingredients (i.e., which may include, but is not limited to absorption enhancers) and adjuvants, carriers, excipients or stabilizers, etc., as defined throughout the instant disclosure.

In some embodiments, the active principal ingredient is a crystalline salt of Compound A, or a pharmaceutically acceptable salt thereof, or a solvate of the foregoing, as described herein. In some embodiments, the active principal ingredient is Form 1, as described herein.

The percentage or amount of active principal ingredient (API) in compositions of the present invention, which may of course, be varied as amount of active compound in such therapeutically useful compositions is such that a suitable dosage for administration in a subject or patient will be obtained. It will be appreciated that the actual preferred dosages of API being used in the compositions of this invention will vary according to the particular composition formulated, the mode of administration, the particular site of administration and the host being treated. The choice of initial dosage most appropriate for the particular patient is determined by the practitioner using well-known medical principles, including, but is not limited to, body weight.

Moreover, an oral tablet dosage form of the present invention may have a surface layer is coated with an enteric coat, which may be, but is not limited to an enteric coating set forth in the Definition section of the instant specification. For example, an oral tablet dosage form may be formulated as with core components, separate sequential layers or combinations thereof, where tablet components, such as core, other layers, may have different release-modifying component properties based upon gastrointestinal environment, pH or time. Hence, an oral tablet dosage form of the present invention may also be coated with a pH sensitive polymer.

Tablets including the compositions of the present invention may be prepared using conventional tablet forming equipment as conventionally known in the art, which may use compaction, rollers and the like.

### VII. Methods of Treatment and/or Uses

In one aspect, the present invention relates to a method and/or use for treating cancer in a subject which comprises administering to the subject a therapeutically effective amount of a crystalline form of Compound A, or a salt thereof, or a solvate of the foregoing. In some variations, the present invention relates to a method and/or use for treating cancer in a subject which comprises administering to the subject a therapeutically effective amount of the crystalline acetate salt of Compound A or solvate or composition thereof disclosed herein.

Provided herein is a method of treating cancer comprising administering the compositions described herein to a subject in need thereof. In some embodiments, the method comprises treating a solid tumor. In some embodiments, the method comprises treating a cancer selected from the group consisting of liver cancer, cholangiocarcinoma, colon cancer, hepatic cholangiocarcinoma, breast, pancreatic, lung, and kidney cancer. In some embodiments, the liver cancer is hepatocellular carcinoma (HCC). In some embodiments the cancers are extrahepatic in origin that metastasizes to and grow in the liver (such as colon, pancreatic, breast, kidney, esophageal, stomach, melanoma and lung). In some embodiments, the subject is human.

The compositions described herein can be administered to a subject in need of treatment for a cell proliferation disorder such as cancer, particularly cancers selected from liver cancer, cholangiocarcinoma, osteosarcoma, melanoma, breast cancer, renal cancer, prostate cancer, gastric cancer, colorectal cancer, thyroid cancer, head and neck cancer, ovarian cancer, pancreatic cancer, neuronal cancer, lung cancer, uterine cancer, leukemia, or lymphoma. The subject is typically a mammal diagnosed as being in need of treatment for one or more of such proliferative disorders, and frequently the subject is a human. The methods comprise administering an effective amount of at least one composition of the invention; optionally the composition may be administered in combination with one or more additional therapeutic agents, particularly the therapeutic agents known to be useful for treating the cancer or proliferative disorder afflicting the particular subject. It would be appreciated by one of ordinary skill in the art that colorectal cancer and colon cancer are used interchangeably and kidney and renal cancer are used interchangeably in this disclosure.

The compositions of the present disclosure are generally administered in a therapeutically effective amount. The term “therapeutically effective amount” may refer to the amount (or dose) of a compound or other therapy that is necessary and sufficient to prevent, reduce, ameliorate, treat or eliminate a condition, or risk thereof, when administered to a subject in need of such compound or other therapy. The amount of the composition administered to a subject may be determined by a physician or caregiver, in the light of the relevant circumstances, including the condition to be treated, the chosen route of administration, the composition administered and its relative activity, the age, weight, the response of the individual patient, the severity of the patient's symptoms, and the like. Thus, the therapeutically effective amount may vary, for example, it may vary depending upon the subject's condition, the weight and age of the subject, the severity of the disease condition, the manner of administration and the like.

The compositions of the current disclosure may be administered by any of the accepted modes of administration of agents having similar utilities, for example, by oral, cutaneous, topical, intradermal, intrathecal, intravenous, subcutaneous, intramuscular, intra-articular, intraspinal or spinal, nasal, epidural, rectal, vaginal or transdermal/transmucosal routes. A suitable route will depend on the nature and severity of the condition being treated. Oral administration may be a primary route of administration for compounds of the present disclosure as they generally exhibit increased oral bioavailability as well as enhanced organ targeting in combination of reduced in vivo toxicity. However, intravenous (IV) administration may be a route of administration for compounds of this disclosure. Intramuscular (IM) administration may be a route of administration for compounds of this disclosure. Subcutaneous, Sublingual, or percutaneous administration can be also contemplated as a route of administration for the compounds of the present disclosure. Sublingual administration may be implemented with an appropriate formulation for the compounds. Inhalation administration can be also employed for a route of administration with an appropriate formulation for the compounds and type of cancer that can be benefited by this route (e.g., lung cancer).

In a particular example, pharmaceutical compositions provided herein may be administered to a human patient orally at a dose about 0.1 mg per kg to about 300 mg per kg or to even 500 mg per kg. In another embodiment, pharmaceutical compositions provided herein may be administered to a human patient orally at a dose about 1 mg per kg to about 300 mg per kg daily. In another particular example, pharmaceutical compositions provided herein may be administered to a human patient orally at a dose about 1 mg per kg to about 100 m per kg.

A subject may suffer from cancer. The subject can be a mammal. The subject can be a human patient suffering from cancer. Examples of cancer include, but are not limited to, adrenal cancer, anal cancer, bile duct cancer, bladder cancer, cancer of the blood, bone cancer, a brain tumor, breast cancer, cancer of the cardiovascular system, cervical cancer, colon cancer, cancer of the digestive system, cancer of the endocrine system, endometrial cancer, esophageal cancer, eye cancer, gallbladder cancer, a gastrointestinal tumor, kidney cancer, laryngeal cancer, leukemia, liver cancer, lung cancer, cholangiocarcinoma, lymphoma, mesothelioma, cancer of the muscular system, myelodysplastic syndrome, myeloma, nasal cavity cancer, nasopharyngeal cancer, cancer of the nervous system, cancer of the lymphatic system, oral cancer, oropharyngeal cancer, ovarian cancer, pancreatic cancer, penile cancer, pituitary tumors, prostate cancer, cancer of the reproductive system, cancer of the respiratory system, a sarcoma, salivary gland cancer, skeletal system cancer, skin cancer, small intestine cancer, stomach cancer, testicular cancer, thymus cancer, thyroid cancer, bladder cancer, or vaginal cancer. In one embodiment, the subject suffers from liver cancer. In another embodiment, the subject suffers from cholangiocarcinoma. In another embodiment, the subject suffers from hepatic cholangiocarcinoma. In yet another embodiment, the subject suffers from kidney cancer. In yet another embodiment, the subject suffers from colon cancer. In yet another embodiment, the subject suffers from lung cancer (e.g., small cell lung cancer or non-small cell lung cancer). In yet another embodiment, the subject suffers from breast cancer. In yet another embodiment, the subject suffers from ovarian cancer. In some embodiments the cancers are extrahepatic in origin that metastasizes to and grow in the liver (such as colon, pancreatic, breast, kidney, esophageal, stomach, melanoma and lung).

Examples of cancer include cancers that cause solid tumors as well as cancers that do not cause solid tumors. Furthermore, any of the cancers mentioned herein may be a primary cancer (e.g., a cancer that is named after the part of the body where it first started to grow) or a secondary or metastatic cancer (e.g., a cancer that has originated from another part of the body).

In some embodiments, provided herein in a method of inhibiting cancer cell proliferation in an individual comprising administering a compound provided herein to the individual. In some embodiments, at least about 10% (including for example at least about any of about 20%, about 30%, about 40%, about 60%, about 70%, about 80%, about 90%, or about 100%) of cell proliferation is inhibited. In some embodiments, proliferation of a solid tumor is inhibited. In some embodiments, the proliferation of liver cancer cells is inhibited. In some embodiments, proliferation of colon cancer cells is inhibited. In some embodiments, proliferation of kidney cancer cells is inhibited. In some embodiments, proliferation of cholangiocarcinoma cells is inhibited.

Also provided herein is a method of inhibiting tumor metastasis in an individual comprising administering a compound provided herein to the individual. In some embodiments, at least about 10% (including for example at least about any of about 20%, about 30%, about 40%, about 60%, about 70%, about 80%, about 90%, or about 100%) of metastasis is inhibited. In some embodiments, metastasis of liver cancer is inhibited. In some embodiments, metastasis of colon cancer is inhibited. In some embodiments, metastasis of kidney cancer is inhibited. In some embodiments, metastasis of cholangiocarcinoma is inhibited. In any of the above embodiments, metastasis to the lymph nodes, lung, bone, or brain is inhibited. In any of the above embodiments, tumor metastasis may be inhibited for at least 1, 2, 3, 4, 5, 6, 7, 8, 9, 10, 11, or 12 weeks following treatment.

In some embodiments, the method comprises reducing tumor size and/or tumor burden in an individual. In some embodiments, the tumor size is reduced at least about 10% (including for example at least about any of about 20%, about 30%, about 40%, about 60%, about 70%, about 80%, about 90%, or about 100%). In some embodiments, the tumor is liver cancer. In some embodiments, the tumor is kidney cancer. In some embodiments, the tumor is colon cancer. In some embodiments, the tumor is a cholangiocarcinoma.

In some embodiments, the method comprises prolonging progression free survival in an individual. In some embodiments, the method prolongs the time to disease progression by at least any of 1, 2, 3, 4, 5, 6, 7, 8, 9, 10, 11, or 12 weeks. In some embodiments, the individual has a solid tumor. In some embodiments, the individual has liver cancer. In some embodiments, the individual has kidney cancer. In some embodiments, the individual has colon cancer. In some embodiments, the individual has a cholangiocarcinoma.

In some embodiments, the method comprises alleviating one or more symptoms in an individual having cancer. In some embodiments, the individual has a solid tumor. In some embodiments, the individual has liver cancer. In some embodiments, the individual has kidney cancer. In some embodiments, the individual has colon cancer. In some embodiments, the individual has a cholangiocarcinoma.

In some embodiments, the method comprises improving the quality of life in an individual having cancer. In some embodiments, the individual has a solid tumor. In some embodiments, the individual has liver cancer. In some embodiments, the individual has kidney cancer. In some embodiments, the individual has colon cancer. In some embodiments, the individual has a cholangiocarcinoma.

In some embodiments, the method results in an objective response (such as a partial response or a complete response) in a patient having cancer. In some embodiments, the individual has a solid tumor. In some embodiments, the individual has liver cancer. In some embodiments, the individual has kidney cancer. In some embodiments, the individual has colon cancer. In some embodiments, the individual has a cholangiocarcinoma.

In some embodiments, compositions of the present invention are not metabolized by cytochrome P-450, resulting in reduced toxicity, in particular hepatotoxicity, compared to existing treatments. Accordingly, in some embodiments, provided herein is a method of treating cancer in an individual, wherein the individual has reduced liver function. In some embodiments, the individual has a Child-Pugh score of Class B or Class C.

In some embodiments, the present methods result in a decrease in one or more markers of liver damage or tumor burden in an individual having liver cancer. In some embodiments, the method results in the level of one or more of alanine amino transferase (ALT), aspartate amino transferase (AST), or alkaline phosphate (ALP) being reduced. In some embodiments, the level of a marker of liver damage is reduced by at least about 5% (such as by about 10%, about, about 15%, about 20%, about 25%, about 30%, about 40%, about 50% about 60%, about 70%, about 80%, or about 90%).

In any of the method of treatment and/or uses embodiments detailed herein, it is understood that such methods and uses may comprise any of the crystalline forms of Compound A and the pharmaceutical compositions as described herein the same as if each and every combination were specifically and individually listed. For example, any of the methods of treatment and/or uses may in some embodiments comprise administering to the subject a therapeutically effective amount of a crystalline form of Compound A, a pharmaceutically acceptable salt thereof, or a solvate of the foregoing. In some embodiments, the crystalline form is selected from an acetate salt of Compound A and a phosphate salt of Compound A. In some embodiments, the crystalline form is selected from Form 1, Form 2, Form 3, Form 4, Form 5, Form 6, Form 7, Form 8, Form 9, Form 10, Form 11, Form 12, and Form 13. In some embodiments, the crystalline form is Form 1.

In some aspects, methods and/or uses of the present invention can comprise administering a crystalline acetate salt of Compound A or solvate or composition thereof of the present invention to a subject in need thereof. In some aspects, the subject in need thereof has been diagnosed with or has been determined to be at risk of developing cancer. In some aspects, the subject is a mammal. In some aspect, the subject is a human.

In some aspects, the present invention provides methods or use of a crystalline acetate salt of Compound A or solvate or composition thereof of the present invention in the manufacture of a medicament for treating cancer.

In some aspects, the method and/or use includes orally administering the crystalline acetate salt of Compound A or solvate or composition thereof of the present invention.

In some aspects, the methods and/or uses of the present invention include administering a crystalline acetate salt of Compound A or solvate or composition thereof in tablet form orally in accordance with patient treatment. In some embodiments, tablets are administered once, twice, or three times daily.

In some aspects, the methods and/or uses of the present invention include administering a dose of a crystalline acetate salt of Compound A or solvate or composition thereof. In some aspects, the acetate salt of Compound A or solvate thereof may be present in any dose range, such as a dose range of from about 1 mg to about 1000 mg, or from about 1 mg to about 500 mg, from about 1 mg to about 100 mg, from about 10 mg to about 50 mg, from about 20 mg to about 40 mg, or from about 20 mg to about 30 mg. In another aspect, the dose range of the acetate salt of Compound A or solvate thereof may be from about 1 mg to about 1000 mg. In another aspect, the dose range of the acetate salt of Compound A or solvate thereof may be from about 5 mg to about 300 mg. In another aspect, the dose range of the acetate salt of Compound A or solvate thereof is from about 25 mg to about 150 mg. In another aspect, the dose range of the acetate salt of Compound A or solvate thereof may be from about 25 mg to about 100 mg. In another aspect, the acetate salt of Compound A or solvate thereof may be present in a dose range of from about 1 mg to about 100 mg. In another aspect, the acetate salt of Compound A or solvate thereof may be present in a dose range of from about 20 mg to about 40 mg. In another aspect, the acetate salt of Compound A or solvate thereof may be present in a dose range of from about 20 mg to about 30 mg.

In yet another aspect, the methods and/or uses of the present invention include administering a dose of a crystalline acetate salt of Compound A or solvate or composition thereof in a dose of about 5 mg, about 10 mg, about 20 mg, about 30 mg, about 40 mg, about 50 mg, about 75 mg, about 100 mg, about 120 mg, or about 150 mg, including any amount in between and fractions thereof. In another aspect, a dose of the acetate salt of Compound A or solvate thereof may be present in about 5 mg. In another aspect, a dose of the acetate salt of Compound A or solvate thereof may be present in about 10 mg. In another aspect, a dose of the acetate salt of Compound A or solvate thereof may be present in about 20 mg. In another aspect, a dose of the acetate salt of Compound A or solvate thereof may be present in about 30 mg. In another aspect, a dose of the acetate salt of Compound A or solvate thereof may be present in about 40 mg. In another aspect, a dose of the acetate salt of Compound A or solvate thereof may be present in about 50 mg. In another aspect, a dose of the acetate salt of Compound A or solvate thereof may be present in about 75 mg. In another aspect, a dose of the acetate salt of Compound A or solvate thereof may be present in about 100 mg. In another aspect, a dose of the acetate salt of Compound A or solvate thereof may be present in about 120 mg. In another aspect, a dose of the acetate salt of Compound A or solvate thereof may be present in about 150 mg.

In some aspects, the methods and/or uses of the present invention include administering a dose of about 10 mg, about 30 mg, or about 120 mg of a crystalline acetate salt of Compound A or solvate or composition thereof once daily or twice daily. In some aspects, the methods and/or uses of the present invention include administering a dose of about 10 mg of a crystalline acetate salt of Compound A or solvate or composition thereof twice daily. In some aspects, the methods and/or uses of the present invention include administering a dose of about 30 mg of a crystalline acetate salt of Compound A or solvate or composition thereof twice daily. In some aspects, the methods and/or uses of the present invention include administering a dose of about 120 mg of a crystalline acetate salt of Compound A or solvate or composition thereof twice daily. In some aspects, the methods and/or uses of the present invention include administering a dose of about 10 mg of a crystalline acetate salt of Compound A or solvate or composition thereof once daily. In some aspects, the methods and/or uses of the present invention include administering a dose of about 30 mg of a crystalline acetate salt of Compound A or solvate or composition thereof once daily. In some aspects, the methods and/or uses of the present invention include administering a dose of about 120 mg of a crystalline acetate salt of Compound A or solvate or composition thereof once daily.

In some embodiments described herein, such as relating to compositions, tablets, capsules, methods, processes, and the like, the acetate salt of Compound A is excluded. In some embodiments described herein, such as relating to compositions, tablets, methods, processes, and the like, the acetate salt of Compound A is included.

In some embodiments described herein, such as relating to compositions, tablets, capsules, methods, processes, and the like, the acetate salt of Compound A is excluded. In some embodiments described herein, such as relating to compositions, tablets, methods, processes, and the like, the acetate salt of Compound A is included.

Each aspect of the present invention defined in this or in any other section may incorporate definitions and limitations, such as those set forth throughout the originally filed disclosure, specification and claims.

### VIII. Examples

In describing the present invention, abbreviations and symbols utilized herein are in accordance with the common usage of such abbreviations and symbols by those skilled in the chemical and biological arts.

### Characterization Methods

### X-Ray Powder Diffraction (XRPD)

XRPD analysis was carried out on a PANalytical X'pert pro with PIXcel detector (128 channels), scanning the samples between 3 and 35° 2θ. The material was gently ground to release any agglomerates and loaded onto a multi-well plate with Kapton or Mylar polymer film to support the sample. The multi-well plate was then placed into the diffractometer and analysed using Cu K radiation (α1 λ=1.54060 A: α2=1.54443 A; β=1.39225 A: α1:α2 ratio=0.5) running in transmission mode (step size 0.0130° 2θ, step time 18.87 s) using 40 kV/40 mA generator settings. Data were visualized and images generated using the HighScore Plus 4.7 desktop application (PANalvtical, 2017).

### Polarised Light Microscopy (PLM)

The presence of crystallinity (birefringence) was determined using an Olympus BX50 microscope, equipped with cross-polarising lenses and a Motic camera. Images were captured using Motic Images Plus 3.0. All images were recorded using the 20× objective, unless otherwise stated.

### Thermogravimetric Analysis Differential Scanning Calorimetry (TGA/DSC)

Approximately, 5-10 mg of material was added into a pre-tared open aluminum pan and loaded into a TA Instruments Discovery SDT 650 Auto—Simultaneous DSC and held at room temperature. The sample was then heated at a rate of 10° C./min from 30° C. to 400° C. during which time the change in sample weight was recorded along with the heat flow response (DSC). Nitrogen was used as the sample purge gas, at a flow rate of 200 cm³/min.

### Differential Scanning Calorimetry (DSC)

Approximately, 1-5 mg of material was weighed into an aluminum DSC pan and sealed nonhermetically with an aluminum lid. The sample pan was then loaded into a TA Instruments Discovery DSC 2500 differential scanning calorimeter equipped with a RC90 cooler. The sample and reference were heated to 170° C. at a scan rate of 10° C./min and the resulting heat flow response monitored. The sample was re-cooled to 20° C. and then reheated again to 275° C. all at 10° C./min. Nitrogen was used as the purge gas, at a flow rate of 50 cm³/min.

### Infrared Spectroscopy (IR)

Infrared spectroscopy was carried out on a Bruker ALPHA P spectrometer. Sufficient material was placed onto the centre of the plate of the spectrometer and the spectra were obtained using the following parameters:
Resolution: 4 cm⁻¹
Background Scan Time: 16 scans
Sample Scan Time: 16 scans
Data Collection: 4000 to 400 cm⁻¹
Result Spectrum: Transmittance
Software: OPUS version 6

### Nuclear Magnetic Resonance (NMR)

¹H NMR experiments were performed on a Bruker AVIIIHD spectrometer equipped with a DCH cryoprobe operating at 500.12 MHz for protons. Experiments were performed in deuterated dimethylsulfoxide (d₆-DMSO) and each sample was prepared to ca. 5-25 mM concentration.

### Dynamic Vapour Sorption (DVS)

Approximately, 10-20 mg of sample was placed into a mesh vapour sorption balance pan and loaded into a DVS Intrinsic dynamic vapour sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH. Two cycles were performed. The weight change during the sorption/desorption cycles were plotted, allowing for the hygroscopic nature of the sample to be determined. XRPD analysis was then carried out on any solid retained.

### High Performance Liquid Chromatography-Ultraviolet Detection (HPLC-UV)

The following parameters were employed:
Instrument: Dionex Ultimate 3000
Column: X-Bridge C18 150×4.6 mm 3.5 μm
Column Temperature: 30° C.
Autosampler Temperature: 5° C.
UV wavelength: 260 nm
Injection Volume: 5.00 μL
Flow Rate: 0.7 mL/min
Mobile Phase A: 0.10% TFA in H₂O
Mobile Phase B: Acetonitrile
Diluent: Water:Methanol v/v 50:50

The following gradient program was used:

| Time (minutes) | Mobile Phase A [%] | Mobile Phase B [%] |
|---|---|---|
| 0 | 85 | 15 |
| 4 | 85 | 15 |
| 20 | 60 | 40 |
| 30 | 20 | 80 |
| 35 | 20 | 80 |
| 35.1 | 85 | 15 |
| 40 | 85 | 15 |

### Liquid Chromatography-Mass Spectrometry (LC-MS)

LC-MS was carried out at the University of Edinburgh. Samples were prepared to a concentration of approximately 20 μg/mL in 1:1 water/acetonitrile 0.1% FA. Analysis was performed using electrospray on a SolariXR FT-ICR MS equipped with a 12T superconducting magnet (Bruker Daltonics).

### Variable Humidity X-Ray Powder Diffraction (VH-XRPD)

XRPD analysis was carried out on a Philips X'Pert Pro Multipurpose diffractometer equipped with a humidity chamber. The samples were scanned between 4 and 36 °2θ using Cu K radiation (α1=1.54060 A; α2=1.54443 A; β=1.39225 A; α1:α2 ratio=0.5) running in Bragg-Brentano geometry (step size 0.008 °2θ) using 40 kV/40 mA generator settings. Measurements were performed at 30° C. during the humidity profile described below:
Initial scan at 40% RH
Humidity increased to 90% RH for 105 minutes (scan after 5 and 105 minutes)
Humidity decreased to 40% RH for 10 minutes (scan after 5 minutes)
Humidity decreased to 5% RH for 60 minutes (scan taken after 60 minutes)
Humidity decreased to 0% RH for 17 hours (scan taken after 17 hours)
Humidity increased to 40% RH for 60 minutes (scan taken at 30 and 60 minutes)
Humidity increased to 60% RH for 105 minutes (scan taken at 5 and 105 minutes)
Humidity increased to 80% RH for 60 minutes (scan taken at 5 and 55 minutes)

### Example 1: Synthesis of Form 1 of a Diacetate Salt of Compound A

Form 1 of 5-((5-(4-carbamimidoylphenoxy)pentyl)oxy)picolinimidamide diacetate (Compound A diacetate salt) was prepared in 6 steps from the starting materials 4-hydroxybenzonitrile (SM-1), 1,5-dibromopentane (SM-2) and 5-hydroxypicolinonitrile (SM-3). The synthetic route is presented in Scheme 1, below.

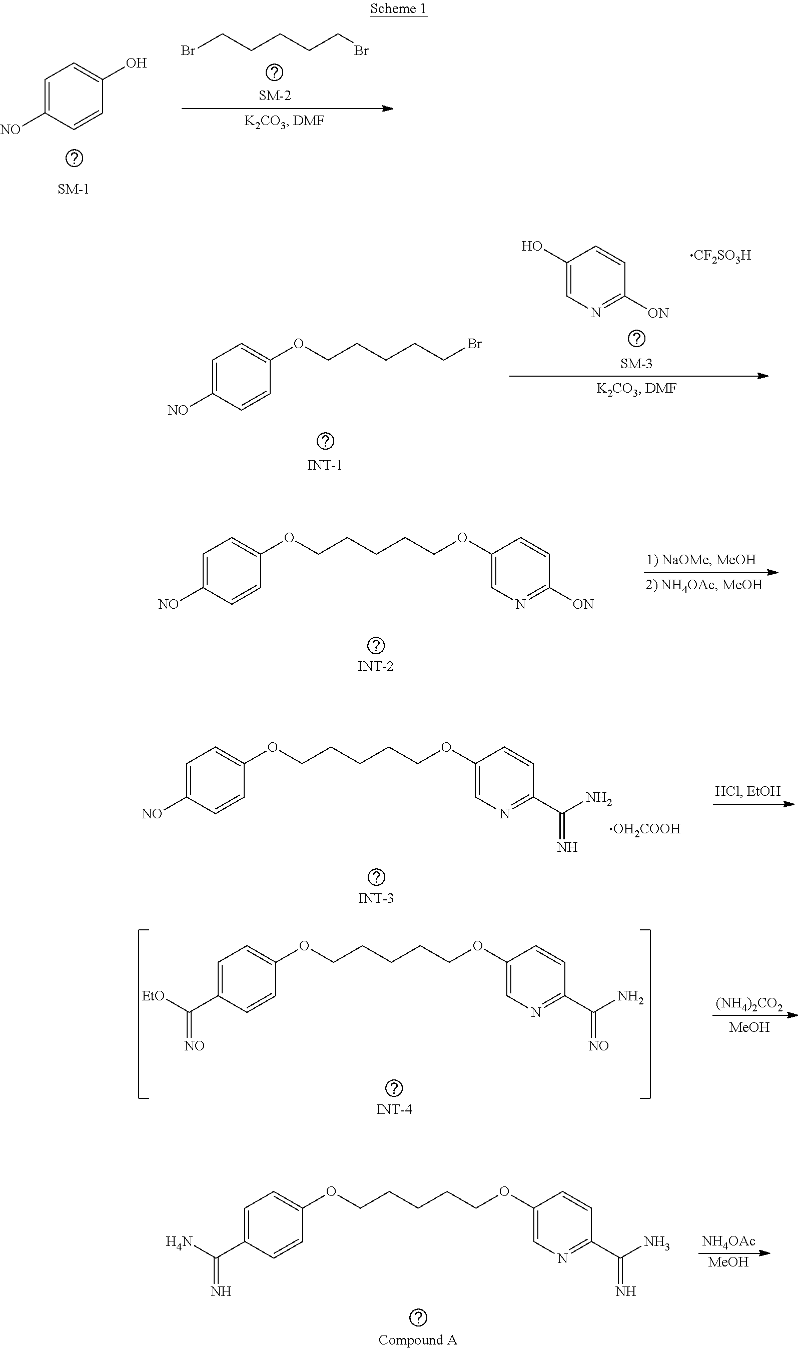

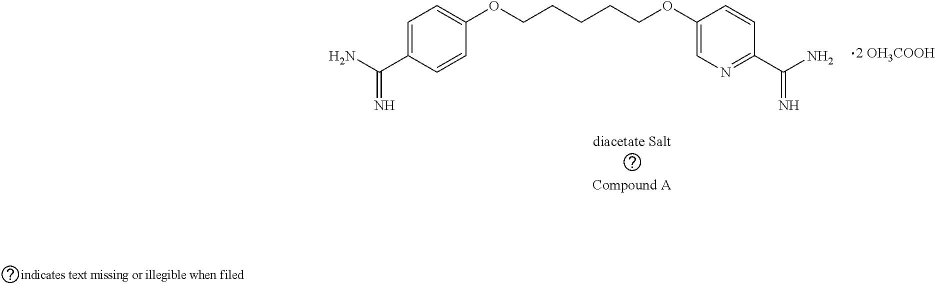 [SMILES: N=C(N)c1ccc(OCCCCCOc2ccc(C(=N)N)nc2)cc1]

In the first step SM-1 is alkylated with SM-2 in the presence of potassium carbonate in N,N-dimethylformamide (DMF) to afford INT-1. SM-3 is then alkylated with INT-1 in the presence of potassium carbonate in DMF to afford INT-2. The nitrile on the 2-position of the pyridine ring is then converted to the amidine by sequential treatment with sodium methoxide and ammonium acetate in methanol to afford INT-3. Treatment of INT-3 with ethanolic HCl affords the imino-ester INT-4 which is not isolated but converted to the corresponding amidine by treatment with ammonium carbonate in methanol to afford Compound A which was converted to the diacetate salt by treatment with ammonium acetate in methanol.

### Step 1: Preparation of 4-((5-bromopentyl)oxy)benzonitrile (INT-1)

To a mixture of SM-2 (22.19 kg, 5.0 eq) and potassium carbonate (3.19 kg, 1.2 eq) in DMF (6.9 L), heated to 40±5° C. was added a solution of SM-1 (2.30 kg, 1.0 eq) in DMF (11.5 L) over approximately 1 hour. The reaction mixture was stirred at 40±5° C. for approximately 4 hours at which time in-process analysis (IPC-1) indicated that the reaction was complete. The reaction mixture was cooled to approximately 30° C. and quenched with water. The biphasic mixture was separated, and the aqueous layer was extracted with n-heptane. The combined organic layers were washed with water then cooled to 25±5° C. The resulting solids were collected by filtration and washed with n-heptane. The filtrate was cooled to −25±5° C. for approximately 3 hours and the resulting solids were collected by filtration and washed with n-heptane. The combined solids were dried at 30±5° C. under vacuum to afford the crude product.

The crude product and n-heptane were combined and heated to 65±5° C. for approximately an hour and then the solution was cooled to 37.5±2.5° C. and filtered. The filtrate, which contains the product, was concentrated by distillation to approximately 5 volumes and then cooled to 30° z 5° C. during which time the product crystallizes. The product was collected by filtration, washed with n-heptane and dried at 30±5° C. under vacuum to afford INT-1 (1.444 kg. 26% yield. 97.8% HPLC purity).

A second batch was conducted with the same process using 0.875 kg SM-1 and 22.19 kg SM-2 which afforded 0.776 kg INT-1 (39% yield, 98.5% HPLC purity).

### Step 2: Preparation of 5-((5-(4-cyanophenoxy)pentyl)oxy)picolinonitrile (INT-2)

To a solution of SM-3 (2.83 kg, 1.17 eq) in DMF (7.20 L) heated to 30±5° C. was added potassium carbonate (3.00 kg, 2.0 eq) in three portions. The resulting mixture was heated to 75±5° C. and then a solution of INT-1 (2.40 kg, 1.0 eq) in DMF (12 L) was added. The reaction mixture was stirred for approximately 1 hour at which time in-process analysis (IPC-1) indicated that the reaction was complete. The reaction mixture was cooled to 30±5° C., quenched with purified water, and stirred at for approximately 2 hours. The solids were collected by filtration, washed with purified water, and dried at 50 to 55° C. under vacuum to afford INT-2 (2.46 kg, 89% yield, 97.3% HPLC purity).

### Step 3: Preparation of 5-((5-(4-cyanophenoxy)pentyl)oxy)picolininmidanide acetate (INT-3)

To a mixture of INT-2 (2.43 kg, 1.0 eq) in methanol (24 L) cooled to 10±5° C. was added a solution of sodium methoxide (0.318 kg, 30% solution in methanol, 0.2 eq) while maintaining the batch temperature of 10±5° C. When the addition was complete the reaction mixture was heated to approximately 50° C. for about 6 hours. The reaction mixture was cooled to 30±5° C. and sampled for in-process analysis (IPC-1). Ammonium acetate (1.82 kg, 3.0 eq) was then added, and the resulting mixture was stirred for about 16 hours at which time in-process analysis showed the reaction to be complete (IPC-2). The reaction mixture was then quenched with purified water and stirred for about 2 hours. The solids were collected by filtration and washed with purified water. The wet solids were slurried in acetone, heated to 50±5° C. for about 2 hours, cooled to 30±5° C., filtered and washed with acetone. This slurry process was repeated two additional times. The wet solids were dried at 55±5° C. under vacuum to afford INT-3 (2.50 kg, 82.5% yield, 97.5% HPLC purity).

### Steps 4 and 5: Preparation of 5-((5-(4-carbamimidoylphenoxy)pentyl)oxy)picolinimidamide (Compound A)

A mixture of INT-3 (2.44 kg) and ethanolic HCl (48.8 L, 5.0 M in ethanol) was cooled to 15-20° C. HCl gas was bubbled into the reaction mixture until HCl content was no less than 32.0% (IPC-1). The reaction mixture was warmed to 30±5° C. and stirred for about 12 hours at which time in-process analysis showed the reaction to be complete (IPC-2). Methyl tert-butyl ether (MTBE) was added to the reaction mixture and the solids (INT-4) were collected by filtration and washed with MTBE. The wet solids were slurried in MTBE, filtered and washed with MTBE to afford INT-4. A mixture of INT-4 and ammonium carbonate (3.17 kg) in methanol (24.5 L) was stirred at 25±5° C. for about 10 hours at which time in-process analysis showed the reaction to be complete (IPC-3). Solvent was removed by distillation until approximately 6 volumes remained. The batch temperature was adjusted to 30±5° C. and acetone was added to the slurry. After about 1 hour the solids were collected by filtration, washed with acetone, and dried at approximately 50-55° C. under vacuum to afford crude Compound A (2.12 kg).

To a solution of the crude Compound A (2.12 kg) in N,N-dimethyl sulfoxide (DMSO, 21.2 L) was added dichloromethane (DCM, 53.1 L) and the resulting mixture was stirred at 30±5° C. for about 5 hours. The solids were collected by filtration, washed with DCM and dried at 50-55° C. under vacuum to afford 1.91 kg of Compound A. Based on results from in-process elemental impurities testing (IPC-4) the batch was further purified in two batches as described below.

To a solution of Compound A (0.945 kg) in methanol (47.25 L) was added activated carbon (Norit® CGP Super, 0.283 kg). The resulting mixture was stirred at 30±5° C. for about 24 hours. The mixture was filtered through a bed of Celite®. The filter bed was washed with two portions of methanol. The combined filtrates were concentrated by distillation until approximately 18 L remained. Acetone was charged and the mixture was stirred at 30±5° C. for approximately 4 hours. The solids were collected by filtration and washed with acetone.

The combined wet cakes from the two batches were slurried in aqueous sodium bicarbonate solution. The solids were collected by filtration and washed with purified water. The wet solids were slurried in acetone, filtered, washed with acetone, and dried at about 30-35° C. under vacuum to afford purified Compound A (1.10 kg. 58% yield, 99.0% HPLC purity).

### Step 6: Preparation of 5-((5-(4-carbamimidoylphenoxy)pentyl)oxy)picolinimidamide diacetate (Form 1 of Compound A Diacetate Salt)

A mixture of Compound A (1.09 kg) and ammonium acetate (0.49 eq) in methanol (10.9 L) was stirred at 30±5° C. for about 12 hours. Methanol (54.5 L) was then added and the batch was warmed to 35±5° C. and stirred for about 2 hours. The reaction mixture was filtered and the filter cake was washed with methanol. The combined filtrates were distilled at approximately 40° C. under vacuum until approximately 20 L remained. Acetone was then added and the resulting slurry was stirred at 30±5° C. for about 45 min. The product was collected by filtration and washed with acetone. The solvent-wet product was slurried in n-heptane at 60-65° C. for about 2 hours. The slurry was cooled to about 30° C. The product was collected by filtration, washed with n-heptane and dried at about 60-70° C. under vacuum to afford Form 1 of the Compound A diacetate salt (0.903 kg, 61% yield, 99.0% HPLC purity).

### Example 2: Characterization of Form 1 of Compound A

Form 1 of the Compound A diacetate salt (“Form 1”) was crystalline by XRPD, as shown in FIG. 1. A summary of the major peaks identified in the XRPD spectrum of Form 1 is provided in Table 1, below.

TABLE 1

| No. | Pos. [°2θ] | Area [cts*°2θ] | Backgr.[cts] | d-spacing [Å] | Height [cts] | Rel. Int. [%] |
|---|---|---|---|---|---|---|
| 1 | 3.1967 | 19.99 | 148.58 | 27.63948 | 198.05 | 8.23 |
| 2 | 5.1963 | 6.72 | 148.58 | 17.00683 | 88.8 | 3.69 |
| 3 | 6.3598 | 8.5 | 148.58 | 13.89786 | 84.15 | 3.5 |
| 4 | 7.1841 | 4.65 | 148.58 | 12.30511 | 36.83 | 1.53 |
| 5 | 8.9291 | 44.89 | 148.58 | 9.90385 | 508.16 | 21.12 |
| 6 | 12.6266 | 72.74 | 164.79 | 7.01073 | 823.46 | 34.23 |
| 7 | 14.5584 | 16.66 | 158.88 | 6.08454 | 66.03 | 2.74 |
| 8 | 15.4335 | 2.71 | 156.2 | 5.74144 | 53.72 | 2.23 |
| 9 | 16.5478 | 11.2 | 152.79 | 5.35725 | 177.56 | 7.38 |
| 10 | 17.2099 | 43.06 | 150.76 | 6.15262 | 284.32 | 11.82 |
| 11 | 17.8642 | 242.86 | 148.76 | 4.96534 | 2405.71 | 100 |
| 12 | 18.8634 | 6.02 | 146.31 | 4.75446 | 79.57 | 3.31 |
| 13 | 19.5435 | 79.79 | 143.62 | 4.54231 | 316.15 | 13.14 |
| 14 | 21.2611 | 5.06 | 138.36 | 4.17907 | 25.07 | 1.04 |
| 15 | 21.827 | 6.21 | 136.63 | 4.07199 | 49.23 | 2.05 |
| 16 | 23.0501 | 11.64 | 132.86 | 3.85862 | 76.89 | 3.2 |
| 17 | 24.2119 | 177.92 | 129.33 | 3.67299 | 1221.98 | 50.79 |
| 18 | 24.292 | 111.11 | 129.08 | 3.67015 | 1335.48 | 55.51 |
| 19 | 24.9406 | 27.04 | 127.1 | 3.5673 | 218.89 | 9.01 |
| 20 | 25.87 | 93.83 | 124.25 | 3.44121 | 1127.75 | 46.88 |
| 21 | 28.6234 | 15.17 | 115.82 | 3.11613 | 30.38 | 1.26 |
| 22 | 30.2918 | 23.94 | 110.71 | 2.9482 | 191.79 | 7.97 |
| 23 | 31.0279 | 17.03 | 108.46 | 2.87991 | 34.12 | 1.42 |
| 24 | 32.0488 | 28.8 | 105.34 | 2.79049 | 115.4 | 4.8 |
| 25 | 33.4576 | 22.07 | 101.02 | 2.67612 | 132.61 | 5.51 |

TG analysis identified a 31.9% mass loss (1.81 equiv. acetic acid) associated with an endothermic event with an onset of 193.1° C. and a peak at 217.7° C., likely corresponding to the melt of Form 1. The sample was seen to degrade above 250° C. (FIG. 2).

The DSC first heat to 170° C. identified a small exothermic event with an onset of 133.1° C. and a peak at 135.2° C. (FIG. 3). No significant thermal events were present in the DSC cool (FIG. 4). The second heat by DSC found a large endothermic event corresponding to the sample melt with an onset of 220.4° C. and a peak at 228.6° C. (FIG. 5).

A second DSC experiment was conducted where the material was heated from 20-275° C. which identified an endothermic event corresponding to the sample melt with an onset of 221.0° C. and a peak at 228.7° C. A sharp exothermic event then closely followed with an onset of 228.9° C. and a peak at 229.9° C., corresponding to re-crystallization of the free form of the API (FIG. 6).

Form 1 was found to be slightly hygroscopic by DVS analysis with a mass uptake of 1.8% at 90% RH (0.5 equiv. of water) (FIG. 7). No indication of any form change was evident in the kinetic plot in FIG. 8. The solid recovered post-DVS analysis remained as the received pattern 1 by XRPD.

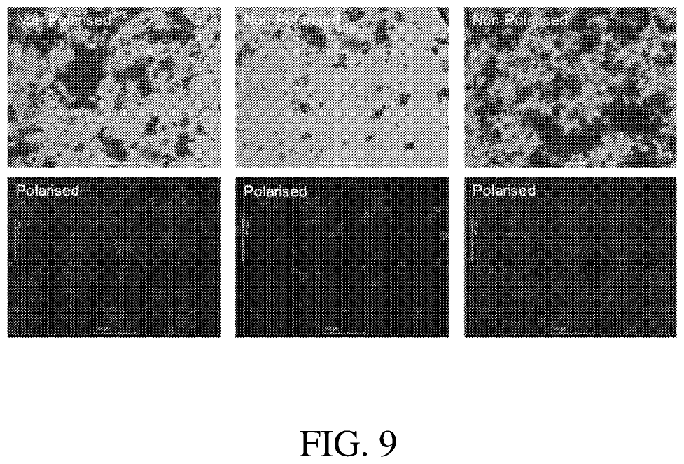

Small, poorly birefringent crystals were seen by PLM but no clear morphology was evident. Agglomeration was also visible (FIG. 9).

The FT-IR spectrum as seen in FIG. 10 was consistent with the expected Compound A structure. The ¹H NMR spectrum is illustrated in FIG. 11 and is consistent with the expected Compound A structure and confirms diacetate salt.

HPLC analysis gave a purity value of 96.7% relative area.

DAD spectrum can be found in FIG. 12 (λmax 264.0 mAU).

LC-MS confirmed molecular weight of the Compound A API to be 342.2 [M+H]+1 with an error of 80 ppb (FIG. 13).

A one week stability assessment of Form 1 found no change in form by XRPD after 2 and 7 days at 40° C./75% RH, 80° C. or ambient temperature and humidity.

The results of the characterization experiments described above are summarized in Table 2, below.

TABLE 2

| Analysis | Result |
|---|---|
| XRPD | Crystalline by XRPD |
| PLM | No clear morphology. Small, birefringent particles and agglomerates visible. |
| TG/DSC | 31.9% mass loss (1.8 equiv. acetic acid) associated with sample melt at 193.1° C. |
| DSC | Small exothermic event in 1st heat at 133.1° C., no events in the DSC cool then |
| | sample melt at 220.4° C. in the 2nd heat. |
| DVS | 1.8% mass uptake at 90% RH (0.5 equiv. of water) indicating the material is |
| | slightly hygroscopic by DVS. No change in form was observed by post-DVS |
| | XRPD. |
| ¹H NMR | Consistent with expected Compound A structure. Confirms diacetate salt. |
| FT-IR | Consistent with expected Compound A structure. Water peak noted at 3248.3 |
| | cm⁻¹. |
| HPLC | % Area purity was calculated to be 96.7%. |
| ESI (+ve) | m/z 342.2 [C18H23N5 + H]1+ (error 80 ppb). |
| LC-MS | |

### Example 3: Solvent Solubility Screen for Compound A Diacetate Salt

Approximately 380 mg of Form 1 was dissolved in 35 mL of methanol and rotary evaporated. The resulting solid was analyzed by XRPD and was found to be a mixture of Form 1 and a new pattern, denoted Form 2. A sub-sample was taken for further characterization (TG/DSC, ¹H NMR and PLM). The crystalline material isolated post rotary-evaporation was re-dissolved in 27 mL of water. The solution remained slightly turbid so was syringe filtered to give a clear solution which was split equally among 18 vials to give approximately 20 mg of material per vial. The samples were then frozen at −50° C. before being lyophilized for ca. 72 hours. The resulting solids were analyzed by XRPD and ¹H NMR and were found to be poorly crystalline. This material was used as input for the solvent solubility screen described below.

The solubility of Compound A diacetate salt was tested in 18 solvent systems. The appropriate solvent was added in 100 μL aliquots to approximately 20 mg of the lyophilized Compound A diacetate salt. Between each addition, the mixture was checked for dissolution and where no dissolution was apparent, the mixture was heated to ca. 40° C. and checked again. This procedure was continued until dissolution was observed or until 100 volumes of solvent had been added.

If the solid did not dissolve after 2 mL of solvent had been added, the slurries were temperature cycled between ambient and 40° C. for 48 hours. The solids were then isolated via centrifugal filtration and analyzed by XRPD. If the samples fully dissolved on addition of solvent (methanol and water only), they were uncapped and left to evaporate at ambient. Any solids recovered post-evaporation were then analyzed by XRPD.

The results of the approximate solvent solubility screen are summarized in Table 3, below. The material was found to have low solubility (<5 mg/mL) in all solvents investigated except from methanol (20 mg/mL) and water (50 mg/mL). XRPD analysis of the recovered solids found 6 novel diffractogram patterns.

TABLE 3

| | | Approximate | |
|---|---|---|---|
| | | Solvent | |
| | | Solubility | Recovered |
| No. | Solvent | (mg/mL) | Form |
| 1 | 1,4-Dioxane | ≤5 | Form 3 and Form 2 |
| 2 | 2-methyl THF | ≤5 | Form 3 |
| 3 | 2-Propanol | ≤5 | Form 3 |
| 4 | Acetone | ≤5 | Form 3 |
| 5 | Acetonitrile | ≤5 | Amorphous |
| 6 | Dichloromethane | ≤5 | Amorphous |
| 7 | Dimethylsulfoxide | ≤5 | Amorphous |
| 8 | Ethanol | ≤5 | Amorphous |
| 9 | Ethyl Acetate | ≤5 | Amorphous |
| 10 | Methylethyl Ketone | ≤5 | Amorphous |
| 11 | Isopropyl Acetate | ≤5 | Amorphous |
| 12 | Methanol | 20 | Amorphous |
| 13 | N,N′-Dimethylacetamide | ≤5 | Form 4 |
| 14 | N,N′-Dimethylformamide | ≤5 | Form 3 |
| 15 | N-Methylpyrrolidone | ≤5 | Form 5 |
| 16 | Tetrahydrofuran | ≤5 | Form 3 and Form 2 |
| 17 | Toluene | ≤5 | Form 6 |
| 18 | Water | 50 | Form 1 and Form 2 |

### Example 4: Primary Polymorph Screen

An amorphous form of Compound A diacetate salt was prepared via lyophilization. Approximately 750 mg of Form 1 of Compound A diacetate salt was dissolved in 78 mL of water. Gentle heating and sonication was used to aid dissolution but the solution remained slightly cloudy so was syringe filtered to give a clear solution. This solution was then split equally among 14 vials to give ca. 50 mg per vial. The samples were then frozen at −50° C. before being lyophilized for ca. 48 hours. A sub-sample was then taken from one sample and analyzed by XRPD. The material was successfully rendered amorphous.

The amorphous form was then added to one of 13 solvents, and subjected to thermal cycling, evaporation, crash cooling, anti-solvent addition, or solvent drop grinding, as described below.

### Thermal Cycling

The appropriate solvent was added in 100 μL aliquots to the lyophilized Compound A diacetate salt material to form a slurry. The samples were then sealed and placed in an incubator shaker to temperature cycle between ambient and 40° C. for ca. 72 hours. Post-thermal cycling, the slurries were centrifuge filtered and the isolated solids analyzed by XRPD. The saturated mother liquor solutions were split equally among four vials for crystallization experiments. The XRPD plate was dried at 40° C. and all samples re-analyzed by XRPD to check for changes in form. The remainder of the isolated solids were dried gently under vacuum at ambient temperature and where new forms were identified, the solids were again analyzed by XRPD prior to ¹H NMR, TG/DSC, PLM and FT-IR analysis.

The results of the thermal cycling experiments are summarized in Table 4. All solids recovered post thermal cycling were crystalline by XRPD analysis. Form 2 was recovered from THF and water but was seen to convert into Form 3 when the THF solids were dried under vacuum and gave a mixture of Form 1 and Form 2 when dried at 40° C. Form 2 solids recovered from water dried to give a new form, Form 9, which was only ever isolated from water, indicating a potential hydrate. Form 3 solids were recovered from thermal cycling in 1,4-dioxane, IPA, acetone and DCM but was seen to change form to Form 11 when solids slurried in DCM were dried at 40° C. for 72 hours. Form 4 was isolated solely from DMA while Form 5 was obtained from NMP, indicating a DMA and NMP solvate. Both forms appeared stable on drying at 40° C. however Form 4 was seen to convert to a mixture of Form 4 and Form 5 on drying under vacuum. Contamination of solvents was seen from ¹H NMR analysis of novel forms upon drying the samples under vacuum and so it is possible Form 4 is a DMA solvate and has been contaminated by NMP in the vacuum oven to give a Form 5 mixture. Form 6, isolated from thermal cycling in toluene, was poorly crystalline by XRPD and remained unchanged on drying at 40° C. Form 7 was recovered from acetonitrile and methanol and did not appear to change form on drying. Form 8 was returned from ethanol and ethyl acetate and although no change in form was seen on drying, a significant reduction in crystallinity was observed on drying the ethanol solids under vacuum.

### Evaporation

Saturated mother liquor solutions, prepared as described in the “Thermal Cycling” section above, were uncapped and left to evaporate at ambient temperature. Observations were recorded post-evaporation and any solids recovered were analyzed by XRPD.

The results of the evaporation experiments are summarized in Table 4. No solids were recovered from the evaporation of the majority of saturated mother liquor solutions, likely due to the low solubility of Compound A diacetate salt in these solvent systems. White solid was recovered from evaporation of the Compound A diacetate salt water solution which was found to be Form 9 by XRPD with the presence of some additional peaks.

### Crash Cooling

Saturated mother liquor solutions, prepared as described in the “Thermal Cycling” section above, were stored at ca. 4° C. for 5 days. Observations were recorded and where possible solids were recovered and analyzed by XRPD. Where no precipitation occurred, or insufficient solids for XRPD analysis were present, the samples were placed into storage at ca. −18° C. for 6 days. Further observations were then recorded and any solids were analyzed by XRPD.

The results of the crash cooling experiments are summarized in Table 4. No precipitation was noted on cooling the saturated mother liquor solutions to 4° C. with the exception of the water sample which returned a cloudy solution. XRPD analysis of the solids recovered post filtration of this solution gave Form 2. Further cooling of the mother liquors at −18° C. gave a small amount of precipitation in the methanol sample, however sufficient material for XRPD analysis could not be recovered.

### Anti-Solvent Addition

Anti-solvent was added to the appropriate saturated mother liquor solutions in 100 μL aliquots until precipitation occurred or 1 mL had been added. Where precipitation was noted, the sample was centrifuge filtered and the recovered solid analyzed by XRPD. Where no precipitation occurred, the samples were placed in a fridge at 4° C. to cool.

Heptane was used as an antisolvent for 1,4-dioxane, 2-propanol, acetone, ethanol, ethyl acetate. N-methylpyrrolidone, tetrahydrofuran, toluene, and DCM. Acetone was used as an antisolvent for acetonitrile, N,N-dimethylacetamide, and water.

The results of the anti-solvent addition experiments are summarized in Table 4. No precipitation was noted on the addition of anti-solvent to the saturated mother liquor solutions with the exception of water which returned amorphous material.

### Solvent Drop Grinding

13×20 mg of Form 1 of Compound A diacetate salt was rendered amorphous via lyophilization for use in the solvent drop grinding experiments. Approximately 260 mg of Form 1 of Compound A diacetate salt was weighed out and dissolved in 26 mL of water. Gentle heating and sonication was used to aid dissolution. The slightly cloudy solution was then syringe filtered to give a clear solution that was split equally among 13 vials. The samples were frozen at −50° C. for ca. 3 hours before being lyophilized for ca. 16 hours. Freeze drying was incomplete after this time so an additional 2 mL of water was added to each sample to re-dissolve the material. The samples were again frozen at −50° C. before being lyophilized for ca. 72 hours. A subsample of sample no. 13 was analyzed by XRPD to ascertain is lyophilization was successful. Four 2.8 mm stainless steel beads were placed in each vial containing the lyophilized Compound A diacetate salt material. 1-2 drops of the appropriate saturated mother liquor solution obtained post thermal cycling was added to each sample vial. The samples were then milled using the following method: Speed: 5000 RPM; Cycle time: 60 seconds; Number of cycles: 5; Time between each cycle: 10 seconds.

The results of the solvent drop grinding experiments are summarized in Table 4. Very poorly crystalline/amorphous material was recovered from IPA, acetone, acetonitrile, ethanol, NMP and water. Form 3 solids were obtained from 1,4-dioxane and THF while Form 4 solids were obtained from DMA. A novel diffractogram, denoted Form 10, was obtained from ethyl acetate, toluene and DCM.

### Preparation of Additional Polymorph Forms

As Form 2 converted to Form 3 and 1 on drying under vacuum at ambient temperature, and Form 4 converted to a mixture of Form 4 and Form 5, attempts were made to re-prepare these forms for further characterization. In addition, Form 8 solids from ethanol lost a significant amount of crystallinity on drying so these solids were also re-processed in an attempt to restore the crystallinity of the material.

For Form 2, 2 mL of THF was added to the Form 3 solids obtained from drying THF solids under vacuum to form a slurry.

For Form 4, 2 mL of DMA was added to the Form 4/Form 5 solids obtained from drying DMA solids under vacuum to form a slurry.

For Form 8, 2 mL of ethanol was added to the poorly crystalline Form 8 material isolated from drying under vacuum to form a slurry.

These slurries were then shaken at 50° C. for ca. 5 hours before being centrifuge filtered and the solids analyzed by XRPD. The crystallinity of the Form 8 material has successfully been restored but no change in form was observed from the THF or DMA slurries. As such, a further 1 mL of DMA was added to the Form 4/Form 5 mixture and a further 2 mL of THF was added to the Form 3 solids. These slurries were then temperature cycled between ambient and 40° C. in 4 hour cycles with agitation for ca. 72 hours. The samples were then centrifuge filtered and again the solids analyzed by XRPD. Again, no change in form was observed with the DMA solids remaining a Form 4/Form 5 mixture and the THF solids remaining as Form 3.

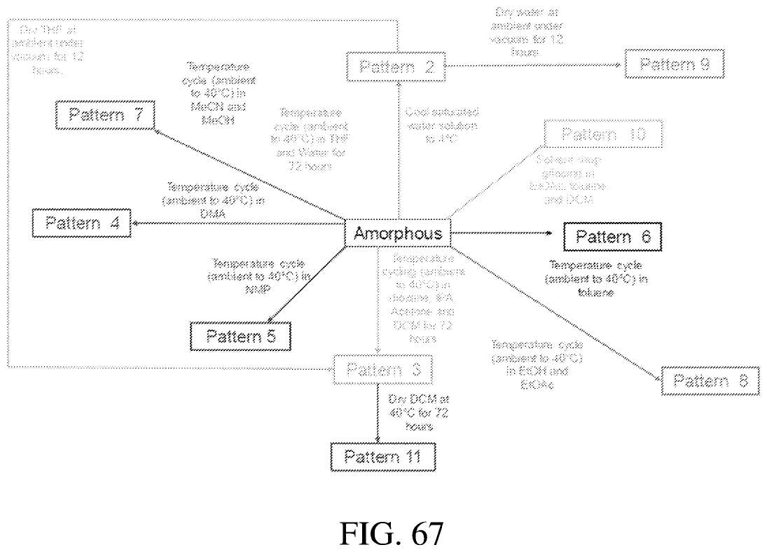

The results of the primary polymorph screen are summarized in Table 4, below. Eleven Compound A diacetate salt polymorphic forms were identified during the primary polymorph screen indicating the diacetate salt has a complex polymorphic nature. Characterization of the novel forms is summarized in Table 5 and a form diagram is presented in FIG. 67.

Form 2 was found to be non-reproducible from THF, unsurprising as it is now known to be a tetrahydrate. Form 3 appeared solvated by TG/DSC and ¹H NMR which both identified 1.0 and 1.2 equivalents of THF respectively within the sample. However, coming from several process relevant solvents (1,4-dioxane, 2-propanol, acetone and THF), it was advised to scale this solvated form up for further investigation. Form 4 was only obtained from DMA and therefore likely a DMA solvate and similarly Form 5 was found to be an NMP solvate with TG/DSC finding 0.8 equivalents of NMP present in the sample. Form 6 was obtained from toluene and gave a poorly crystalline diffractogram and gradual 13.6% (0.7 equiv. toluene) mass loss from the onset of heating by TG/DSC.

Form 7 was obtained from methanol and acetonitrile. ¹H NMR found no methanol in the solids analyzed and so TG/DSC analysis identified a sharp 4.5% mass loss corresponding to 1.2 equivalents of water, making Form 7 a monohydrate. Form 8 was obtained from ethanol and ethyl acetate and originally believed to be an anhydrous form as there was no mass loss on heating the material by TG/DSC. Salt disproportionation/sample melt was seen at 186.1° C., lower than that of Form 1, meaning Form 8 was originally believed to be an anhydrous metastable form of Compound A diacetate salt. It was later concluded during the secondary screen that Form 8 was another solvated form.

Form 9 was obtained from water and was scaled up during the secondary screen for this reason. TG/DSC analysis found 1.7 equivalents of water within the sample and large, highly birefringent, lath-like morphology was present by PLM. Form 10 was only observed from solvent drop grinding experiments while Form 11 was only obtained on drying Form 3 from DCM.

TABLE 4

| | | Thermal Cycling | | | | Crash Cooling | | Anti-solvent | Solvent Drop |
|---|---|---|---|---|---|---|---|---|---|
| No. | Solvent | Wet | Dry (40° C.) | Dry (vac) | Evaporation | 4° C. | 18° C. | Addition | Grinding |
| 1 | 1,4-Dioxane | Form 3 | Form 3* | N/A | No Solid | Frozen | N/A | Clear Solution | Form 3 |
| 2 | 2-Propanol | Form 3 | Form 3 | N/A | No Solid | Clear Solution | Clear Solution | Clear Solution | Amorphous |
| 3 | Acetone | Form 3 | Form 3*/2 | N/A | No Solid | Clear Solution | Clear Solution | Clear Solution | Amorphous |
| 4 | Acetonitrile | Form 7 | Form 7 | N/A | No Solid | Clear Solution | Clear Solution | Clear Solution | Amorphous |
| 5 | Ethanol | Form 8 | Form 8 | Form 8* | Insufficient | Clear Solution | Clear Solution | Clear Solution | Amorphous |
| | | | | | Solid | | | | |
| 6 | Ethyl Acetate | Form 8 | Form 8 | N/A | No Solid | Clear Solution | Clear Solution | Clear Solution | Form 10 |
| 7 | Methanol | Form 7 | Form 7 | Form 7 | N/A | Clear Solution | Insufficient | N/A | N/A |
| | | | | | | | Solid | | |
| 8 | N,N′- | Form 4 | Form 4 | Form 4/5 | Clear Solution | Clear Solution | Clear Solution | Clear Solution | Form 4 |
| | Dimethylacetamide | | | | | | | | |
| 9 | N- | Form 5 | Form 5 | Form 5 | Clear Solution | Clear Solution | Clear Solution | Clear Solution | Amorphous |
| | Methylpyrrolidone | | | | | | | | |
| 10 | THF | Form 2 | Form 1/2 | Form 3 | No Solid | Clear Solution | Clear Solution | Clear Solution | Form 3 |
| 11 | Toluene | Form 6* | Form 6* | N/A | Clear Solution | Clear Solution | Clear Solution | Clear Solution | Form 10 |
| 12 | Water | Form 2 | Form 9 | Form 9 | Form 9 | Form 2 | N/A | Amorphous | Amorphous |
| 13 | DCM | Form 3 | Form 11 | N/A | No Solid | Clear Solution | Clear Solution | Clear Solution | Form 10 |

*Poorly crystalline

TABLE 6

| | | | | ¹H NMR | | | |
|---|---|---|---|---|---|---|---|
| XRPD | | Crystal- | | Counterion | | | |
| Pattern | Solvents | linity | TG/DSC | Equiv. | Solvent Equiv. | PLM | Comments |
| Pattern | N/A | Good | melt 193.1° C. | Diacetate | <0.1 hexane | Unclear, poorly | As Received |
| 1 | | | | | | birefringent | |
| Pattern | THF, Water | Poor | N/A | N/A | N/A | N/A | Non- |
| 2 | | | | | | | Reproducible |
| | | | | | | | from THF |
| Pattern | Dioxane, | Poor | 14.9% mass loss, melt | Diacetate | 1.2 THF, 0.6 DCM, 0.3 DMA, | Unclear, | Appears |
| 3 | IPA, | | 183.7° C. | | 0.3 NMP, 0.3 IPA | birefringent | Solvated |
| | Acetone, | | | | | | |
| | THF | | | | | | |
| Pattern | DMA | Average | N/A | N/A | N/A | N/A | Possible DMA |
| 4 | | | | | | | solvate |
| Pattern | NMP | Good | 16.2% mass loss, melt | Diacetate | 2.6 NMP, <0.1 Dioxane, 0.1 | Unclear, | NMP Solvate |
| 5 | | | 190.2° C. | | DMA, <0.1 THF, 0.2 Hexane | birefringent | |
| Pattern | Toluene | Poor | 13.6% mass loss, melt | Diacetate | 2.2 Toluene, <0.1 Dioxane, | Unclear, | Toluene |
| 6 | | | 190.5° C. | | 0.1 DMA, 0.1 NMP, <0.1 IPA, | birefringent | Solvate |
| | | | | | 0.1 Hexane | | |
| Pattern | Acetonitrile, | Good | 8.5% mass loss then 4.5% | Diacetate | 0.1 Dioxane and DCM, 0.8 | Unclear, highly | Hydrate, |
| 7 | Methanol | | mass loss (1.2 equiv. H₂O) | | DMA, 0.2 NMP and EtOAc, <0.1 | birefringent | dehydrates |
| | | | melt 194.5° C. | | IPA, Toluene and Acetone | | to pattern 1 |
| Pattern | Ethanol, | Good | Anhydrous, melt 186.0° C. | Diacetate | Large amount of EtOH present | Unclear, poorly | Anhydrous, |
| 8 | Ethyl | | | | | birefringent | metastable |
| | Acetate | | | | | | form |
| Pattern | Water | Average | 6.7% mass loss (1.7 equiv. | Diacetate | <0.1 Dioxane and IPA, 0.1 | Lath-like | Hydrate, |
| 9 | | | H₂O), melt 191.6° C. | | DMA, NMP and hexane | morphology, | dehydrates |
| | | | | | | highly | to pattern 1 |
| | | | | | | birefringent | |
| Pattern | Ethyl | Good | N/A | N/A | N/A | N/A | Only observed |
| 10 | Acetate, | | | | | | from solvent |
| | Toluene, | | | | | | drop grading |
| | DCM | | | | | | |
| Pattern | DCM | Average | N/A | N/A | N/A | N/A | Obtained from |
| 11 | | | | | | | drying DCM at |
| | | | | | | | 40° C. |

### Characterization of Novel Polymorphs

### Form 2

The XRPD diffractogram for Form 2 is presented in FIG. 14.

### Form 3

Form 3 was recovered from thermal cycling amorphous Compound A diacetate salt in 1,4-dioxane. IPA, acetone and DCM. It was also recovered post drying of Form 2 material under vacuum from THF and solvent drop grinding using a saturated THF solution. The XRPD diffractogram is presented in FIG. 15 with a peak list given in Table 6. The Form 3 material obtained post drying of Form 2 material from THF was used for further characterization. ¹H NMR identified 1.2 equivalents of THF with a 4.89H peak at 3.60 ppm. The material was consistent with the Compound A diacetate structure with a 6.00H peak at 1.74 ppm corresponding to the CH₃ of acetic acid (FIG. 16).

TABLE 6

| No. | Pos. [°2θ] | Area [cts*°2θ] | Backgr.[cts] | d-spacing [Å] | Height [cts] | Rel. Int. [%] |
|---|---|---|---|---|---|---|
| 1 | 4.2446 | 53.85 | 210.80 | 20.81755 | 88.9 | 11.89 |
| 2 | 4.6928 | 3.64 | 210.89 | 18.83031 | 96.15 | 12.86 |
| 3 | 8.9726 | 0.12 | 161.44 | 9.85591 | 1.63 | 0.22 |
| 4 | 9.9226 | 84.89 | 160.18 | 8.91429 | 747.5 | 100 |
| 5 | 10.9588 | 21.29 | 158.81 | 8.07369 | 281.25 | 37.63 |
| 6 | 13.0718 | 2.16 | 156.01 | 6.77294 | 42.84 | 5.73 |
| 7 | 14.0282 | 16.88 | 154.74 | 6.31327 | 33.44 | 4.47 |
| 8 | 16.3978 | 18.33 | 151.6 | 5.40591 | 181.58 | 24.29 |
| 9 | 17.8393 | 53.22 | 149.69 | 4.96809 | 365.51 | 48.9 |
| 10 | 17.9264 | 24.37 | 149.58 | 4.95643 | 234.28 | 31.34 |
| 11 | 19.2197 | 62.74 | 147.86 | 4.61427 | 251.35 | 33.63 |
| 12 | 19.9644 | 64.47 | 146.88 | 4.44382 | 221.4 | 29.62 |
| 13 | 20.6277 | 70.62 | 146 | 4.30239 | 242.5 | 32.44 |
| 14 | 21.7932 | 50.78 | 144.46 | 4.07485 | 152.58 | 20.41 |
| 15 | 22.3967 | 34.44 | 143.66 | 3.9664 | 206.98 | 27.69 |
| 16 | 23.7066 | 20.51 | 141.92 | 3.75012 | 61.62 | 8.24 |
| 17 | 25.1219 | 104.38 | 140.05 | 3.54196 | 716.91 | 95.91 |
| 18 | 26.9414 | 13.14 | 137.64 | 3.30674 | 78.94 | 10.56 |
| 19 | 28.843 | 21.93 | 135.12 | 3.09291 | 43.94 | 5.88 |
| 20 | 29.5234 | 17.34 | 134.22 | 3.02316 | 52.11 | 6.97 |
| 21 | 29.8832 | 12.77 | 133.74 | 2.98758 | 51.15 | 6.84 |
| 22 | 31.6244 | 7.23 | 135.79 | 2.82694 | 21.72 | 2.91 |
| 23 | 32.483 | 9.09 | 139.76 | 2.75415 | 30.4 | 4.87 |
| 24 | 33.3118 | 8.15 | 143.59 | 2.6875 | 32.64 | 4.37 |

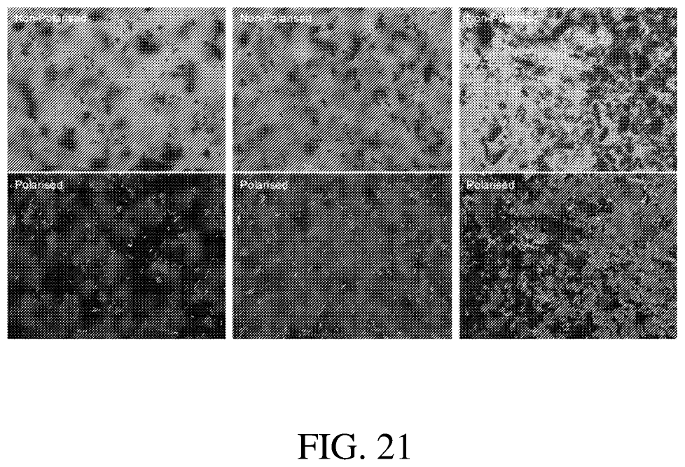

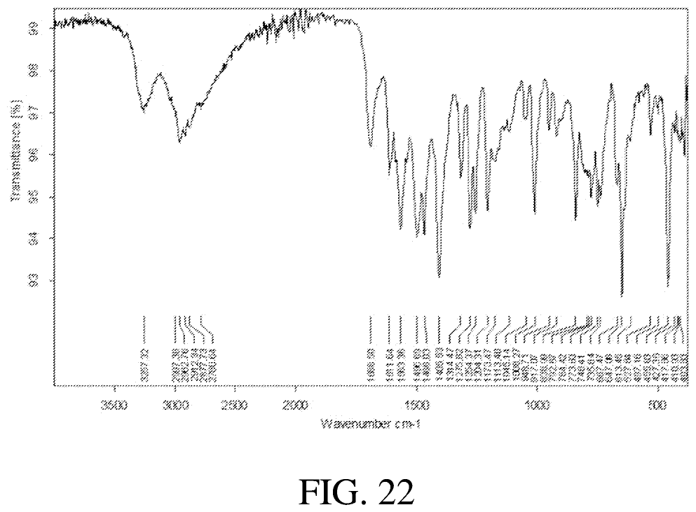

TG/DSC analysis of Form 3 identified a mass loss of 14.9% (0.95 equiv. of THF) associated with a broad endothermic event with an onset of 74.0° C. and a peak at 125.6° C., likely due to the desolvation of the material. A broad endothermic event associated with sample melt then occurs, similar to that of Form 1, with an onset of 183.7° C. and a peak at 197.8° C. with an associated weight loss of 27.9% (1.6 equiv. acetic acid). This indicates possible de-solvation to Form 1 (FIG. 17-20, 25). PLM analysis found no clear particle morphology but the presence of birefringence was noted. Agglomeration was also visible (FIG. 21). The FT-IR spectrum is presented in FIG. 22. The presence of water is evident from the peak at 3255.68 cm-1 (peak list given in Table 7).

TABLE 7

| Wavenumber (cm⁻¹) | Rel. Intensity |
|---|---|
| 3255.6766 | 0.042 |
| 2950.3321 | 0.101 |
| 1601.3957 | 0.072 |
| 1811.798 | 0.052 |
| 1562.5093 | 0.116 |
| 1497.1013 | 0.125 |
| 1465.6276 | 0.066 |
| 1405.4086 | 0.259 |
| 1315.2425 | 0.096 |
| 1276.081 | 0.155 |
| 1254.5842 | 0.089 |
| 1205.2429 | 0.154 |
| 1178.3113 | 0.031 |
| 1115.1096 | 0.034 |
| 1067.2831 | 0.059 |
| 1050.2793 | 0.03 |
| 1008.8635 | 0.155 |
| 914.4118 | 0.063 |
| 838.0146 | 0.183 |
| 790.1215 | 0.023 |
| 773.9859 | 0.078 |
| 748.5042 | 0.139 |
| 735.6929 | 0.042 |
| 668.139 | 0.076 |
| 646.8696 | 0.327 |
| 611.0461 | 0.021 |
| 529.9707 | 0.062 |
| 506.1072 | 0.01 |
| 456.1848 | 0.27 |
| 427.4637 | 0.014 |
| 413.4419 | 0.038 |

### Form 4

The XRPD diffractogram for Form 4 is presented in FIG. 26.

### Form 5

Form 5 was recovered from thermal cycling amorphous Compound A diacetate salt in NMP. The XRPD diffractogram is illustrated in FIG. 27 with a peak list given in Table 8. ¹H NMR found 2.6 equivalents of NMP with a 7.72H CH₃ peak at 2.70 ppm. <0.1 equivalents of 1,4-dioxane and THF, 0.1 equivalents of DMA and 0.2 equivalents of hexane were also present in the NMR spectrum due to cross contamination of solvent on drying the solids in a vacuum oven. The material was consistent with the Compound A diacetate structure with a 6.00H peak at 1.74 ppm corresponding to the CH₃ of acetic acid (FIG. 28).

TABLE 8

| No. | Pos. [°2θ] | Area [cts*°2θ] | Backgr.[cts] | d-spacing [Å] | Height [cts] | Rel. Int. [%] |
|---|---|---|---|---|---|---|
| 1 | 3.0965 | 6.32 | 222.57 | 26.54287 | 20.68 | 1.01 |
| 2 | 7.3039 | 4.13 | 184.51 | 12.10355 | 27.25 | 1.32 |
| 3 | 8.7822 | 49.69 | 184.18 | 10.06922 | 787.61 | 38.07 |
| 4 | 9.0458 | 26.32 | 184.18 | 9.77638 | 417.16 | 20.16 |
| 5 | 9.616 | 8.52 | 184.18 | 9.19787 | 84.42 | 4.08 |
| 6 | 10.0584 | 63.95 | 184.18 | 8.79428 | 844.71 | 40.63 |
| 7 | 10.3195 | 3.99 | 184.18 | 8.57236 | 52.65 | 2.54 |
| 8 | 10.6414 | 3.1 | 184.18 | 8.31376 | 40.93 | 1.98 |
| 9 | 10.7975 | 3.76 | 184.18 | 8.1939 | 49.63 | 2.4 |
| 10 | 11.5462 | 7.03 | 184.18 | 7.66421 | 92.9 | 4.49 |
| 11 | 11.995 | 43.31 | 184.18 | 7.37842 | 686.61 | 33.18 |
| 12 | 13.6128 | 12.39 | 184.18 | 6.41123 | 245.4 | 11.86 |
| 13 | 14.9066 | 50.21 | 184.18 | 5.94317 | 663.11 | 32.05 |
| 14 | 16.0215 | 24.84 | 184.18 | 5.53203 | 656.25 | 31.72 |
| 15 | 16.8793 | 72.92 | 184.18 | 5.25279 | 825.56 | 39.91 |
| 16 | 17.3302 | 122.87 | 184.18 | 5.1171 | 1391.05 | 67.24 |
| 17 | 17.6481 | 26.52 | 184.18 | 5.02565 | 525.35 | 25.99 |
| 18 | 18.1419 | 182.74 | 184.18 | 4.88995 | 2068.77 | 100 |
| 19 | 18.6845 | 130.89 | 184.18 | 4.74916 | 1152.56 | 55.71 |
| 20 | 19.5967 | 60.78 | 184.18 | 4.5301 | 602.09 | 29.1 |
| 21 | 19.8517 | 47.55 | 184.18 | 4.47247 | 471.07 | 22.77 |
| 22 | 21.4041 | 54.65 | 184.18 | 4.15148 | 619.09 | 29.93 |
| 23 | 22.4378 | 41.98 | 184.18 | 3.9625 | 415.81 | 20.1 |
| 24 | 22.8546 | 166.59 | 184.18 | 3.89118 | 1466.89 | 70.91 |
| 25 | 23.906 | 30.5 | 184.18 | 3.72236 | 302.14 | 14.6 |
| 26 | 24.8112 | 147.4 | 184.18 | 3.58858 | 1297.93 | 62.74 |
| 27 | 25.2272 | 169.08 | 184.18 | 3.53034 | 1486.79 | 71.96 |
| 28 | 26.4792 | 31.76 | 184.18 | 9.36619 | 314.57 | 15.21 |
| 29 | 27.8054 | 39.41 | 184.18 | 3.20858 | 195.19 | 9.43 |
| 30 | 26.3711 | 17.86 | 184.18 | 3.14587 | 283.09 | 13.68 |
| 31 | 28.6915 | 13.43 | 184.18 | 3.11147 | 354.81 | 17.15 |
| 32 | 29.0081 | 20.31 | 184.18 | 3.07823 | 268.21 | 12.96 |
| 33 | 30.4056 | 20.41 | 184.18 | 2.93985 | 134.76 | 6.51 |
| 34 | 30.8848 | 17.28 | 184.18 | 2.89534 | 273.93 | 13.24 |
| 35 | 33.1832 | 36.62 | 184.18 | 2.69385 | 241.84 | 11.69 |
| 36 | 34.3317 | 15.85 | 184.18 | 2.60996 | 253.94 | 12.27 |
| 37 | 34.5123 | 30.12 | 184.18 | 2.59887 | 238.68 | 11.54 |

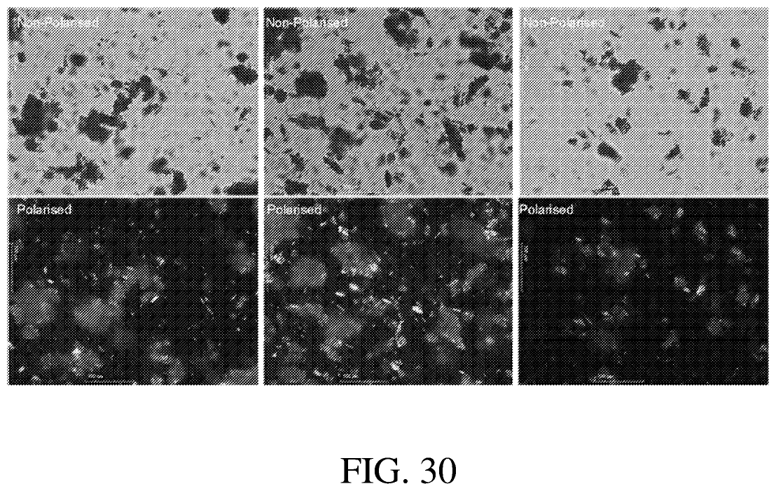

TG/DSC analysis identified a 18.2% mass loss (0.84 equiv. of NMP) associated with a small endothermic event with an onset of 145.4° C. and a peak at 161.0° C. A large endothermic event corresponding to sample melt then occurs with an onset of 190.2° C. and a peak at 204.1° C. with an associated 24.4% mass loss (1.4 equiv. acetic acid). This indicates de-solvation to Form 1 (FIG. 29). PLM analysis found no clear morphology, but birefringent particles and agglomerates were present (FIG. 30). FT-IR was consistent with the Compound A diacetate structure as expected from the ¹H NMR data (FIG. 31).

### Form 6

Form 6 was isolated post-thermal cycling amorphous Compound A diacetate salt material in toluene. The XRPD diffractogram is illustrated in FIG. 32 with a peak list given in Table 9. ¹H NMR found 2.2 equivalents of toluene present in the sample with a 6.5H peak at 2.30 ppm. <0.1 equivalents of dioxane and IPA and 0.1 equivalents of DMA, NMP and hexane were also present in the NMR spectrum (FIG. 33). The material was consistent with the Compound A diacetate structure with a 6.00H peak at 1.74 ppm.

TABLE 9

| No. | Pos. [°2θ] | Area [cts*°2θ] | Backgr.[cts] | d-spacing [Å] | Height [cts] | Rel. Int. [%] |
|---|---|---|---|---|---|---|
| 1 | 5.6904 | 187.97 | 211.4 | 15.53139 | 1354.23 | 100 |
| 2 | 7.4999 | 1.32 | 161.84 | 11.78756 | 6.53 | 0.48 |
| 3 | 9.0887 | 7.97 | 154.58 | 9.73027 | 105.23 | 7.77 |
| 4 | 9.5349 | 19.62 | 154.01 | 9.27593 | 155.5 | 11.48 |
| 5 | 11.4096 | 7.55 | 157.02 | 7.75566 | 74.82 | 5.52 |
| 6 | 12.5161 | 11.28 | 163.12 | 7.07241 | 74.48 | 5.5 |
| 7 | 17.054 | 22.66 | 176.18 | 5.19936 | 74.82 | 5.52 |
| 8 | 17.5331 | 18.88 | 172.34 | 5.05835 | 93.49 | 6.9 |
| 9 | 18.2714 | 18.85 | 166.43 | 4.8556 | 373.42 | 27.57 |
| 10 | 18.4887 | 35.42 | 164.69 | 4.799 | 467.82 | 34.55 |
| 11 | 20.1801 | 5.06 | 151.15 | 4.40043 | 25.07 | 1.86 |
| 12 | 21.8337 | 4.15 | 147.6 | 4.07076 | 32.91 | 2.43 |
| 13 | 22.4764 | 51.67 | 148.2 | 3.95579 | 292.5 | 21.6 |
| 14 | 24.7984 | 26.24 | 150.39 | 3.5904 | 86.65 | 8.4 |
| 15 | 26.0531 | 35.1 | 151.57 | 3.42027 | 115.91 | 8.56 |
| 16 | 26.5658 | 45.63 | 152.65 | 3.35542 | 258.27 | 19.07 |
| 17 | 31.3369 | 2.95 | 129.4 | 2.55458 | 29.26 | 2.16 |
| 18 | 32.5215 | 1.71 | 127.42 | 2.75326 | 4.24 | 0.31 |

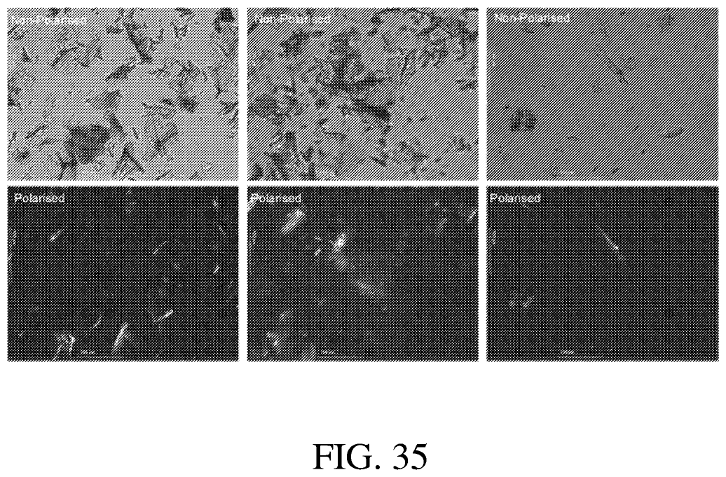

TG/DSC analysis identified a gradual mass loss of 13.6% (0.7 equiv. of toluene) associated with a broad endothermic event with an onset of 58.2° C. and a peak at 113.6° C., likely corresponding to desolvation. The sample melt then occurs with an onset of 190.6° C. and a peak at 203.9° C. with an associated mass loss of 21.2% (1.2 equiv. of acetic acid). This indicates de-solvation to Form 1 due to the similar melt of 193.1° C. (FIG. 34). PLM images are presented in FIG. 35. No clear morphology was evident but birefringent particles and agglomerates were present. The FT-IR spectrum was consistent with the expected diacetate structure (FIG. 36).

### Form 7

Form 7 was obtained from temperature cycling amorphous Compound A diacetate salt material in acetonitrile and methanol. The XRPD diffractogram is presented in FIG. 37 with a peak list given in Table 10. ¹H NMR analysis found no methanol to remain in the sample post drying under vacuum however 0.1 equivalents of DCM and dioxane, 0.8 equivalents of DMA, 0.2 equivalents of NMP and ethyl acetate and <0.1 equivalents of IPA, toluene, and acetone were all present in the spectrum. The material was also found to be consistent with the diacetate structure confirming no salt disproportionation had occurred (FIG. 38).

TABLE 10

| No. | Pos. [°2θ] | Area [cts*°2θ] | Backgr.[cts] | d-spacing [Å] | Height [cts] | Rel. Int. [%] |
|---|---|---|---|---|---|---|
| 1 | 5.1087 | 70.41 | 203.3 | 17.29828 | 1115.92 | 40.05 |
| 2 | 5.6134 | 0.86 | 186.28 | 15.74421 | 13.71 | 0.49 |
| 3 | 10.1362 | 210.95 | 143.35 | 8.72697 | 2786.23 | 100 |
| 4 | 10.4265 | 130.66 | 143.35 | 8.48465 | 1150.51 | 41.29 |
| 5 | 10.6607 | 19.15 | 143.35 | 8.29875 | 303.5 | 10.89 |
| 6 | 11.3637 | 15.4 | 143.35 | 7.78689 | 406.93 | 14.6 |
| 7 | 11.5103 | 19.86 | 143.35 | 7.68801 | 524.67 | 18.83 |
| 8 | 11.6747 | 56.8 | 143.35 | 7.58016 | 643.05 | 23.08 |
| 9 | 12.7372 | 11.86 | 143.35 | 6.95009 | 134.31 | 4.82 |
| 10 | 13.0192 | 7.3 | 143.35 | 6.80022 | 96.44 | 3.46 |
| 11 | 14.3972 | 15.76 | 143.35 | 6.15229 | 312.32 | 11.21 |
| 12 | 14.8513 | 4.25 | 143.35 | 5.96518 | 56.19 | 2.02 |
| 13 | 15.3999 | 20.70 | 143.35 | 5.75391 | 68.6 | 2.45 |
| 14 | 16.4208 | 12.93 | 143.35 | 5.39838 | 204.87 | 7.35 |
| 15 | 16.6467 | 26.92 | 143.35 | 5.32122 | 323.58 | 11.61 |
| 16 | 16.7178 | 16.23 | 143.35 | 5.31192 | 250.06 | 9.33 |
| 17 | 17.7524 | 53.44 | 143.35 | 4.99222 | 428.24 | 15.37 |
| 18 | 17.9449 | 155.83 | 143.35 | 4.93909 | 749.18 | 26.89 |
| 19 | 18.4299 | 42.04 | 143.35 | 4.81019 | 505.25 | 18.73 |
| 20 | 18.5083 | 88.73 | 143.35 | 4.79 | 710.95 | 25.52 |
| 21 | 18.7792 | 34.17 | 143.35 | 4.72151 | 328.6 | 11.79 |
| 22 | 20.3597 | 65.93 | 143.35 | 4.36841 | 528.29 | 18.96 |
| 23 | 20.6204 | 160.49 | 143.35 | 4.32464 | 964.48 | 34.52 |
| 24 | 21.0321 | 282.7 | 143.35 | 4.22056 | 1235.59 | 44.35 |
| 25 | 21.8055 | 26.17 | 143.35 | 4.07259 | 419.37 | 15.05 |
| 26 | 22.0923 | 33.48 | 143.35 | 4.02035 | 402.43 | 14.44 |
| 27 | 22.4226 | 55.04 | 143.35 | 3.96188 | 264.6 | 3.5 |
| 28 | 22.7017 | 5.75 | 143.35 | 3.91379 | 138.17 | 4.96 |
| 29 | 22.8773 | 50.37 | 143.35 | 3.88415 | 201.8 | 7.24 |
| 30 | 23.4679 | 84.24 | 143.35 | 3.78772 | 404.98 | 14.53 |
| 31 | 23.7026 | 35.23 | 143.35 | 3.75074 | 282.31 | 10.13 |
| 32 | 24.1585 | 132.39 | 143.35 | 3.68095 | 1060.85 | 38.07 |
| 33 | 24.7744 | 540.98 | 143.35 | 3.59085 | 2000.67 | 71.81 |
| 34 | 25.2874 | 46.78 | 143.35 | 3.51915 | 224.92 | 8.07 |
| 35 | 26.2699 | 621.02 | 143.35 | 3.38973 | 2714.23 | 97.42 |
| 36 | 27.2182 | 31.24 | 143.35 | 3.27374 | 250.34 | 8.98 |
| 37 | 27.4644 | 78.77 | 143.35 | 3.24494 | 295.54 | 10.61 |
| 38 | 27.9591 | 53.57 | 143.35 | 3.18865 | 429.22 | 15.41 |
| 39 | 28.2391 | 27.75 | 143.35 | 3.15767 | 222.36 | 7.98 |
| 40 | 29.1359 | 57.53 | 143.35 | 3.06248 | 172.86 | 6.2 |
| 41 | 29.5639 | 21.77 | 143.35 | 3.01911 | 130.65 | 4.7 |
| 42 | 30.1954 | 68.77 | 143.35 | 2.95739 | 275.53 | 9.89 |
| 43 | 31.1076 | 53.65 | 143.35 | 2.87272 | 257.95 | 9.26 |
| 44 | 31.4344 | 18.95 | 143.35 | 2.84359 | 182.19 | 6.54 |
| 45 | 31.7841 | 37.1 | 143.35 | 2.8131 | 178.37 | 6.4 |
| 46 | 33.1681 | 16.34 | 143.35 | 2.69881 | 261.9 | 9.4 |
| 47 | 33.9234 | 68.31 | 143.35 | 2.64043 | 136.83 | 4.91 |
| 48 | 34.7491 | 18.36 | 143.35 | 2.57955 | 147.1 | 5.28 |

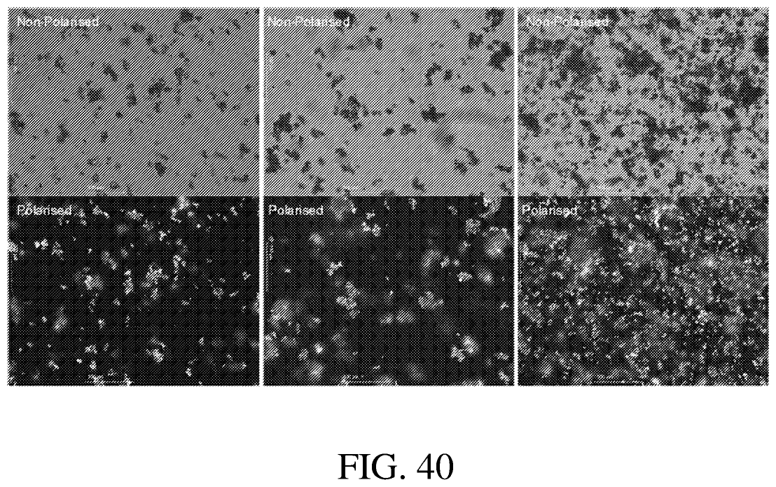

TG analysis identified a gradual 7.5% mass loss prior to a sharp 4.6% mass loss (1.2 equivalents of water) associated with a small endothermic event with an onset of 147.7° C. and a peak at 147.9° C. The sample then melts with an onset of 195.1° C. and a peak at 213.0° C. with a mass loss of 26.2% (1.5 equiv. acetic acid) corresponding to salt disproportionation (FIG. 39). PLM images are illustrated in FIG. 40 with small, highly birefringent particles visible. No clear crystal morphology was identified. FT-IR analysis was consistent with the expected diacetate structure with a water peak evident at 3263.89 cm-1 (FIG. 41). A FT-IR peak list is given in Table 11.

TABLE 11

| Wavenumber (cm⁻¹) | Rel. Intensity |
|---|---|
| 3263.8881 | 0.017 |
| 2949.921 | 0.042 |
| 2874.9139 | 0.003 |
| 1692.5437 | 0.023 |
| 1631.9449 | 0.005 |
| 1610.9948 | 0.02 |
| 1563.8372 | 0.04 |
| 1537.5083 | 0.004 |
| 1495.6468 | 0.044 |
| 1468.5722 | 0.016 |
| 1402.5501 | 0.08 |
| 1335.9096 | 0.003 |
| 1313.3134 | 0.029 |
| 1272.6386 | 0.02 |
| 1253.1489 | 0.046 |
| 1200.3311 | 0.037 |
| 1170.4312 | 0.011 |
| 1112.6934 | 0.012 |
| 1038.2526 | 0.013 |
| 1008.155 | 0.05 |
| 917.4091 | 0.017 |
| 834.2445 | 0.026 |
| 785.1443 | 0.006 |
| 771.4092 | 0.023 |
| 740.8762 | 0.051 |
| 665.4149 | 0.008 |
| 647.1254 | 0.084 |
| 611.2152 | 0.009 |
| 591.7189 | 0.006 |
| 561.6084 | 0.005 |
| 550.4058 | 0.004 |
| 529.0548 | 0.017 |
| 512.5004 | 0.002 |
| 501.991 | 0.004 |
| 457.3076 | 0.084 |
| 429.951 | 0.005 |
| 411.1117 | 0.012 |

### Form 8

Form 8 was obtained from temperature cycling amorphous Compound A diacetate salt in ethanol and ethyl acetate. The XRPD diffractogram is illustrated in FIG. 42 with a peak list given in Table 12. The ¹H NMR spectrum is presented in FIG. 43 which confirms the diacetate structure. A large amount of ethanol is visible as the material was not dried pre-NMR analysis due to the loss in crystallinity observed on drying.

TABLE 12

| No. | Pos. [°2θ] | Area [cts*°2θ] | Backgr.[cts] | d-spacing [Å] | Height [cts] | Rel. Int. [%] |
|---|---|---|---|---|---|---|
| 1 | 5.4968 | 306.1 | 237.16 | 16.0778 | 4042.93 | 100 |
| 2 | 9.1831 | 81.06 | 166.58 | 9.63046 | 1070.69 | 26.48 |
| 3 | 10.1011 | 13.43 | 166.58 | 8.75724 | 212.9 | 5.27 |
| 4 | 10.2736 | 21.82 | 166.58 | 8.61053 | 345.77 | 8.56 |
| 5 | 11.1207 | 57.78 | 166.58 | 7.95652 | 596.86 | 14.76 |
| 6 | 11.3253 | 9.59 | 166.58 | 7.81323 | 125.69 | 3.13 |
| 7 | 13.7351 | 13.19 | 166.58 | 6.44734 | 174.18 | 4.31 |
| 8 | 14.9785 | 14.94 | 166.58 | 5.9148 | 236.79 | 5.86 |
| 9 | 15.274 | 9.39 | 166.58 | 5.80103 | 74.4 | 1.84 |
| 10 | 16.5799 | 9.43 | 166.58 | 5.34695 | 93.39 | 2.31 |
| 11 | 17.8177 | 9.71 | 166.58 | 4.9782 | 192.31 | 4.76 |
| 12 | 18.5824 | 55.51 | 166.58 | 4.77502 | 1099.76 | 27.2 |
| 13 | 18.7605 | 236.72 | 166.58 | 4.73009 | 2344.98 | 58 |
| 14 | 19.1766 | 9.42 | 166.58 | 4.62637 | 186.64 | 4.62 |
| 15 | 20.1846 | 28.05 | 166.58 | 4.39945 | 222.32 | 5.5 |
| 16 | 21.2538 | 36.94 | 166.58 | 4.18049 | 243.97 | 6.03 |
| 17 | 21.5109 | 11.85 | 166.58 | 4.13111 | 313.04 | 7.74 |
| 18 | 21.8514 | 16.23 | 166.58 | 4.0675 | 214.41 | 5.3 |
| 19 | 22.126 | 21.36 | 166.58 | 4.01764 | 211.56 | 5.23 |
| 20 | 22.4169 | 27.15 | 166.58 | 3.95615 | 537.94 | 13.31 |
| 21 | 23.2239 | 4.3 | 166.58 | 3.82697 | 103.41 | 2.56 |
| 22 | 23.8106 | 54.53 | 166.58 | 3.73707 | 452.16 | 10.69 |
| 23 | 24.395 | 218.99 | 166.58 | 3.64685 | 1334.96 | 33.02 |
| 24 | 24.7684 | 25.14 | 166.58 | 3.59468 | 498.05 | 12.32 |
| 25 | 24.9994 | 41.33 | 166.58 | 3.56199 | 327.56 | 8.1 |
| 26 | 25.6136 | 19.01 | 166.58 | 3.47794 | 251.05 | 6.21 |
| 27 | 25.6974 | 28.07 | 166.58 | 3.44048 | 222.47 | 5.5 |
| 28 | 26.3031 | 136.88 | 166.58 | 3.38833 | 1084.71 | 26.83 |
| 29 | 27.2764 | 36.27 | 166.58 | 3.26959 | 478.99 | 11.85 |
| 30 | 27.8436 | 8.8 | 166.58 | 3.20426 | 58.13 | 1.44 |
| 31 | 28.4719 | 21.92 | 166.58 | 3.13496 | 248.11 | 6.14 |
| 32 | 29.229 | 6.29 | 166.58 | 3.05546 | 41.08 | 1.02 |
| 33 | 30.1238 | 2.28 | 166.58 | 2.96571 | 36.09 | 0.8 |
| 34 | 30.6959 | 13.14 | 166.58 | 2.91272 | 260.32 | 6.44 |
| 35 | 31.1111 | 3.62 | 166.58 | 2.87241 | 86.94 | 2.15 |
| 36 | 31.3874 | 10 | 166.58 | 2.8501 | 49.54 | 1.23 |
| 37 | 32.584 | 4.92 | 166.58 | 2.74612 | 32.52 | 0.89 |
| 38 | 33.7931 | 16.31 | 166.58 | 2.65251 | 80.78 | 2 |

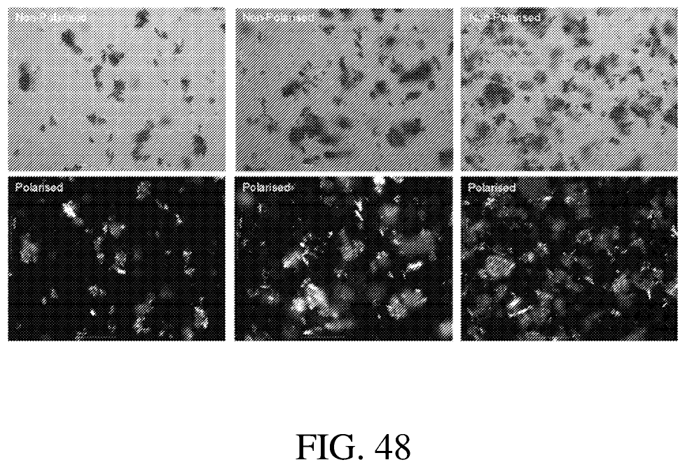

The Compound A material appears anhydrous by TG/DSC with no weight loss identified until a 28.6% (1.6 equiv. of acetic acid) mass loss associated with sample melt with an onset of 186.0° C. and a peak at 203.7° C. (FIG. 44-47, 52). PLM images are illustrated in FIG. 48 where small, poorly birefringent particles are visible. The FT-IR spectrum was consistent with the expected diacetate structure and is presented in FIG. 49 with a peak list given in Table 13.

TABLE 13

| Wavenumber (cm⁻¹) | Rel. Intensity |
|---|---|
| 2943.1049 | 0.172 |
| 1676.5606 | 0.034 |
| 1609.6933 | 0.072 |
| 1568.556 | 0.106 |
| 1487.672 | 0.168 |
| 1465.4245 | 0.036 |
| 1402.8248 | 0.326 |
| 1334.5077 | 0.022 |
| 1313.1334 | 0.082 |
| 1267.4437 | 0.166 |
| 1246.58 | 0.021 |
| 1196.9616 | 0.151 |
| 1159.4377 | 0.08 |
| 1118.4303 | 0.051 |
| 1089.2673 | 0.014 |
| 1049.6534 | 0.02 |
| 1033.8031 | 0.11 |
| 1012.2767 | 0.05 |
| 986.909 | 0.022 |
| 923.813 | 0.085 |
| 881.0811 | 0.013 |
| 836.6356 | 0.178 |
| 773.4458 | 0.042 |
| 745.4353 | 0.108 |
| 666.3482 | 0.039 |
| 647.0442 | 0.242 |
| 612.7709 | 0.014 |
| 522.0713 | 0.034 |
| 495.9315 | 0.022 |
| 465.2627 | 0.035 |
| 450.7477 | 0.013 |
| 426.6152 | 0.083 |

### Form 9

Form 9 was recovered post drying of Form 2 from water and on evaporation of a saturated water solution, indicating possible hydration. The XRPD diffractogram is presented in FIG. 53 with a peak list given in Table 14. ¹H NMR was consistent with the diacetate salt structure with <0.1 equivalents of dioxane and IPA and 0.1 equivalent of DMA, NMP and hexane present (FIG. 54). TG/DSC analysis identified an initial 6.7% mass loss (1.7 equiv. of water) around 50° C., likely corresponding to dehydration, prior to sample melt/salt disproprtionation with a 30.9% (1.8 equiv. acetic acid) mass loss associated with an endothermic event with onset 191.6° C. and peak at 206.7° C. (FIG. 55-58, 63). This indicates Form 9 dehydrates to Form 1 readily.

TABLE 14

| No. | Pos. [°2θ] | Area [cts*°2θ] | Backgr.[cts] | d-spacing [Å] | Height [cts] | Rel. Int. [%] |
|---|---|---|---|---|---|---|
| 1 | 3.9416 | 8.53 | 141.24 | 22.41744 | 112.62 | 8.07 |
| 2 | 4.877 | 10.87 | 141.24 | 18.11955 | 107.72 | 7.72 |
| 3 | 6.362 | 17.82 | 141.24 | 13.89326 | 235.35 | 16.87 |
| 4 | 6.7615 | 19.32 | 141.24 | 13.07308 | 47.84 | 3.43 |
| 5 | 6.9987 | 9.73 | 141.24 | 12.63068 | 257.15 | 18.44 |
| 6 | 7.0821 | 15 | 141.24 | 12.48197 | 237.7 | 17.04 |
| 7 | 10.4232 | 28.95 | 130.35 | 8.48726 | 458.82 | 32.89 |
| 8 | 11.4497 | 3.06 | 130.35 | 7.7286 | 30.35 | 2.18 |
| 9 | 12.5322 | 25.53 | 130.35 | 7.06337 | 337.19 | 24.17 |
| 10 | 12.7509 | 7.36 | 130.35 | 6.93726 | 116.64 | 8.36 |
| 11 | 12.9663 | 2.91 | 130.35 | 6.82783 | 46.1 | 3.31 |
| 12 | 14.0419 | 22.73 | 130.35 | 6.30713 | 300.22 | 21.52 |
| 13 | 14.1807 | 34.33 | 130.35 | 5.24571 | 340.08 | 24.38 |
| 14 | 14.6423 | 12.75 | 130.35 | 5.96879 | 252.62 | 18.11 |
| 15 | 16.2405 | 37.09 | 130.35 | 5.45791 | 73.48 | 5.27 |
| 16 | 17.1575 | 7.75 | 130.35 | 5.16822 | 122.81 | 8.8 |
| 17 | 17.502 | 6.69 | 130.35 | 5.06726 | 88.34 | 6.33 |
| 18 | 17.9028 | 4.97 | 130.35 | 4.95472 | 131.33 | 9.42 |
| 19 | 18.6746 | 13.65 | 130.35 | 4.75164 | 216.38 | 15.51 |
| 20 | 19.4274 | 140.81 | 130.35 | 4.5692 | 1394.86 | 100 |
| 21 | 20.8029 | 51.77 | 130.35 | 4.27007 | 512.87 | 36.77 |
| 22 | 21.3467 | 73.82 | 130.35 | 4.16251 | 835.73 | 59.92 |
| 23 | 23.2637 | 11.58 | 130.35 | 3.82367 | 229.36 | 16.44 |
| 24 | 23.6641 | 32.33 | 130.35 | 3.75987 | 427.04 | 30.62 |
| 25 | 24.1571 | 62.21 | 130.35 | 3.68425 | 704.29 | 50.49 |
| 26 | 25.0183 | 69.69 | 130.35 | 3.55934 | 460.21 | 32.99 |
| 27 | 25.2329 | 36.21 | 130.35 | 3.52955 | 358.73 | 25.72 |
| 28 | 26.1417 | 43.41 | 130.35 | 3.40868 | 343.98 | 24.66 |
| 29 | 26.5715 | 23.67 | 130.35 | 3.3547 | 156.35 | 11.21 |
| 30 | 27.7037 | 12.5 | 130.35 | 3.22013 | 123.82 | 8.88 |
| 31 | 28.126 | 20.67 | 130.35 | 3.17273 | 163.8 | 11.74 |
| 32 | 28.5325 | 36.38 | 130.35 | 3.12845 | 205.95 | 14.76 |
| 33 | 29.1344 | 13.17 | 130.35 | 3.06517 | 173.95 | 12.47 |
| 34 | 29.7099 | 16.65 | 130.35 | 3.00709 | 164.94 | 11.82 |
| 35 | 30.1944 | 22.95 | 130.35 | 2.95994 | 151.56 | 10.87 |
| 36 | 30.9877 | 25.05 | 130.35 | 2.88595 | 165.48 | 11.86 |
| 37 | 31.4562 | 20.89 | 130.35 | 2.84402 | 165.55 | 11.87 |
| 38 | 31.7378 | 13.61 | 130.35 | 2.81943 | 107.82 | 7.73 |
| 39 | 32.0077 | 8.44 | 130.35 | 2.79627 | 133.7 | 9.59 |
| 40 | 32.5089 | 19.48 | 130.35 | 2.75429 | 154.38 | 11.07 |
| 41 | 32.8651 | 30.42 | 130.35 | 2.72525 | 150.68 | 10.8 |
| 42 | 33.812 | 18.71 | 130.35 | 2.65107 | 185.37 | 13.29 |
| 43 | 34.7376 | 21.1 | 130.35 | 2.58252 | 208.98 | 14.98 |

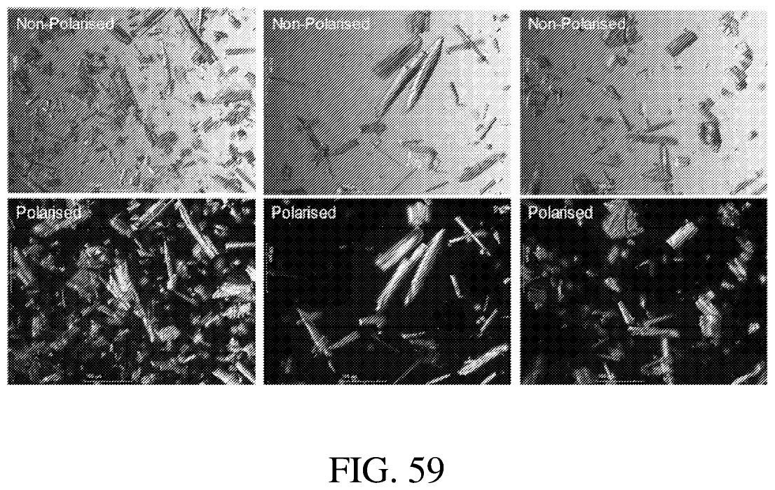

PLM analysis identified large, highly birefringent, lath-like morphology with agglomeration visible (FIG. 59). The FT-IR spectrum is presented in FIG. 60 which was consistent with the expected diacetate structure with a water peak visible at 3232.61 cm-1. An FT-IR peak list is provided in Table 15.

TABLE 15

| Wavenumber (cm⁻¹) | Rel. Intensity |
|---|---|
| 3232.6127 | 0.028 |
| 2945.6972 | 0.112 |
| 2872.5212 | 0.008 |
| 1689.1529 | 0.038 |
| 1608.9923 | 0.038 |
| 1564.4854 | 0.06 |
| 1489.1865 | 0.023 |
| 1464.537 | 0.066 |
| 1398.4721 | 0.16 |
| 1311.1014 | 0.058 |
| 1253.6221 | 0.098 |
| 1202.1281 | 0.021 |
| 1179.8662 | 0.051 |
| 1113.2871 | 0.013 |
| 1067.5152 | 0.012 |
| 1042.7806 | 0.022 |
| 1008.2217 | 0.086 |
| 917.9675 | 0.027 |
| 837.5159 | 0.103 |
| 790.0759 | 0.008 |
| 772.2314 | 0.023 |
| 737.0262 | 0.058 |
| 667.4188 | 0.018 |
| 647.1573 | 0.147 |
| 612.1907 | 0.012 |
| 547.8813 | 0.016 |
| 528.0807 | 0.011 |
| 453.097 | 0.112 |
| 423.1608 | 0.009 |

### Form 11

The XRPD diffractogram for Form 11 is presented in FIG. 64.

### Example 5: Secondary Polymorph Screen

Crystalline forms 3, 8, and 9 were scaled up for further characterization.

Approximately 1.2 g of Form 1 of Compound A diacetate salt was weighed out and dissolved in 126 mL of water. The solution was gently heated and sonicated to aid dissolution before being syringe filtered and split equally among 3 duran bottles to give approximately 400 mg of material per vial. The samples were then frozen at −50° C. before being lyophilized over 4 days. A subsample was analyzed by XRPD post-lyophilization to ascertain if amorphization had been successful.

### Preparation and Characterization of Form 3

25.6 mL of 2-propanol was added to the lyophilized Compound A diacetate salt to form a slurry. The slurry was then temperature cycled between ambient and 40° C. in 4 hour cycles with agitation for ca. 24 hours. The slurry was then filtered and the isolated solids collected into a scintillation vial. A subsample was analyzed by XRPD and the vial was left uncapped to allow the solids to div at ambient for ca. 24 hours. The material was then re-analyzed by XRPD post-drying and fully characterized using the following techniques: TG/DSC, DSC, DVS with post-DVS XRPD analysis, PLM, FT-IR, ¹H NMR, Purity by HPLC, TG/DSC with post TG/DSC XRPD analysis, and 7 Day Stability Assessment (ca. 10 mg of the material was stored at 40° C./75% RH. 80° C. and ambient light and humidity for 1 week. The solids were then analyzed by XRPD and purity obtained by HPLC). Thermodynamic solubility in water and a pH 7.4 phosphate buffer (The appropriate media was added in 100 μL aliquots to ca. 20 mg of the material to form a slurry. The samples were then sealed and agitated at ambient for ca. 24 hours before being centrifuge filtered. The isolated solids were analyzed by XRPD and the concentration of the mother liquors determined by HPLC).

Form 3 was scaled up successfully as confirmed by XRPD and retained form and crystallinity on drying at ambient for ca. 24 hours. TG/DSC confirmed the removal of surface solvent and identified a two-step mass loss of 11.7% (0.9 equiv. IPA) associated with a small endothermic event with an onset of 129.1° C. and a peak at 126.6° C., likely corresponding to desolvation/dehydration. A large mass loss of 29.4% (1.7 equiv. acetic acid) occurs with a large endothermic event with onset of 192.2° C. and a peak at 209.8° C. associated with salt disproportionation/Form 1 melt (FIG. 17). A large endothermic event was present in the first heat by DSC with an onset of 120.9° C. and a peak at 143.6° C. corresponding to desolvation (FIG. 18). No significant thermal events were then present in the DSC cool or 2nd heat (FIG. 19-20).

No clear crystal morphology was visible by PLM analysis however small, highly birefringent particles were present (FIG. 21). ¹H NMR found 1.4 equivalents of IPA in the sample with a 8.6H peak at 1.04 ppm (FIG. 16). The FT-IR spectrum is consistent with the expected diacetate structure and a water peak is present at 3257.32 cm⁻¹ in FIG. 22.

DVS analysis of Form 3 found the material to convert to the hydrated Form 9 at 50% RH with a loss of approximately 4 wt. % (loss of 0.3 equiv. IPA or 1.0 equiv. water). Form 9 is hygroscopic by DVS with a mass uptake of 8% at 90% RH (2.0 equiv. of water) with dehydration below 10% RH (FIG. 23). The conversion of Form 3 to Form 9 is evident in the DVS kinetic plot as the weight of the sample stabilizes over time at the 50-60% RH stage (FIG. 24). The recovered solids post-DVS analysis were analyzed by XRPD, which confirmed conversion to Form 9.

The results of the one-week stability assessment of Form 3 are summarized in Table 16. The material was seen to convert to Form 1 after 1 week at 40° C./75% RH and under ambient temperature and humidity however no change was seen after 1 week at 80° C. The material was heated by TG/DSC to 150° C. causing an 11.0% weight loss (0.8 equiv. IPA or 3.2 equiv. water) with an associated endothermic event with an onset of 132.5° C. and a peak at 137.9° C. (FIG. 25), confirming desolvation. The recovered solids were then analyzed by XRPD which confirmed conversion to Form 1.

Form 3 is a solvated form of Compound A diacetate salt which readily converts to the thermodynamically stable Form 1 on desolvation as seen from the one-week stability assessment and desolvation/XRPD experiment. It also converts to the hydrated Form 9 under high humidity as seen in the DVS analysis.

In summary, Form 3 was solvated with TG/DSC identifying an 11.7% mass loss (0.9 equiv. IPA or 3.0 equiv. water) with a melt at 192.2° C. and ¹H NMR identifying 1.4 equivalents of IPA. DSC identified an endothermic event at 120.9° C. corresponding to desolvation and PLM analysis of the material found no clear morphology but small, highly birefringent particles were visible. On desolvation above 150° C., Form 3 was found to convert to Form 1. Similarly, on storage of the Form 3 material at 40° C./75% RH and under ambient temperature and humidity, the material was found to convert to Form 1. DVS analysis (FIG. 23-24) saw the material convert to Form 9 (dihydrate) at 50% RH. Form 3 therefore appears to be a low risk solvate as it readily converts to Form 1.

### Preparation and Characterization of Form 8

Form 8 was scaled up using the following procedure with amorphous input material prepared according to the process described above. 27.2 mL of ethanol was added to the lyophilized Compound A diacetate salt to form a slurry. The slurry was then temperature cycled between ambient and 40° C. in 4 hour cycles with agitation for ca. 24 hours. The slurry was then filtered via Buchner filtration and the isolated solids collected into a scintillation vial. A subsample was analyzed by XRPD and the XRPD plate was then placed in an oven at 40° C. for 1 hour and the sample re-analyzed by XRPD to check for loss in crystallinity on drying at elevated temperature. The vial was left uncapped to allow the solids to dry at ambient for ca. 24 hours before being re-analyzed by XRPD. Solids were characterized using the same techniques used for Form 3, above.

Form 8 was scaled up successfully and retained form on drying at ambient for ca. 24 hours. Significant loss in crystallinity was observed on drying solids on the XRPD plate at 40° C. for 1 hour and therefore the bulk material was not dried at elevated temperature. TG analysis identified a gradual 6.7% weight loss (0.7 equiv. of ethanol) from the onset of heating. A large endothermic event then occurs associated with a further 30.6% weight loss with an onset of 196.0° C. and a peak at 218.5° C., corresponding the salt disproportionation/melt (FIG. 44). Two broad endothermic events were present in the 1st heat by DSC due to the gradual drying of the material. The first event has an onset of 32.5° C. and a peak at 55.6° C. and the second event has an onset of 78.8° C. and a peak at 103.2° C. (FIG. 45). No significant thermal events were then present in the DSC cool or 2nd heat (FIG. 46-47).

No clear crystal morphology was visible by PLM analysis but small, highly birefringent particles were present (FIG. 48). ¹H NMR confirms the diacetate structure and found 1.8 equivalents of ethanol in the sample with a 5.51H peak at 1.06 ppm (FIG. 43). The FT-IR spectrum is consistent with the expected structure. A shoulder can be seen in the broad peak ca. 3000 cm⁻¹, potentially due to a water peak (FIG. 49).

DVS analysis of the Form 8 material found the material to desolvate and convert to Form 1 at 50% RH via loss of 8.2 wt. % (0.9 equiv. ethanol) as seen in the isotherm plot in Figure FIG. 50. The Form 1 material is then slightly hygroscopic with a mass increase of 1.8% at 90% RH (consistent with the initial characterization). The change in form is evident in the kinetic plot in FIG. 51 which shows the sample weight to take a significant amount of time to stabilise initially before proceeding rapidly through the DVS cycle after conversion to Form 1. Post-DVS XRPD confirmed the conversion of Form 8 to Form 1.

The results of the one-week stability assessment are summarized in Table 16. Like the Form 3 material, the Form 8 solids were seen to convert to Form 1 on storage at 40° C./75% RH and under ambient temperature and humidity. Although no change in form was observed on storage at 80° C., a significant loss in crystallinity was observed. The Form 8 material was heated to 150° C. prior to XRPD analysis. TG/DSC identified a two-step mass loss of 2.3% (0.2 equiv. ethanol or 0.6 equiv. water) followed by 8.6% (1.0 equiv. ethanol or 2.2 equiv. water) with two associated endothermic events at 34.2° C. and 67.0° C. (FIG. 52). XRPD analysis of the recovered solids found the material to decrease significantly in crystallinity but was not seen to convert to Form 1 on desolvation as with Form 3.

Based on the results obtained, Form 8 appears to be a metastable solvated form of Compound A diacetate salt which readily converts to Form 1 as seen during the one-week stability assessment and DVS analysis under elevated humidity and ambient conditions. On drying, the material becomes poorly crystalline but does not convert to Form 1.

In summary, Form 8 was also found to be a solvated form of the diacetate salt with TG/DSC identifying a 6.7% mass loss (0.7 equiv. ethanol or 1.7 equiv. of water) with a melt at 196.0° C. ¹H NMR also identified 1.8 equivalents of ethanol present within the sample. Two broad endotherms were seen by DSC at 32.5° C. and 78.8° C. corresponding to the desolvation of the sample. No clear morphology was present by PLM but small, highly birefringent, particles were visible. Drying the material to 150° C. does not convert this solvate to Form 1. However, the material did convert to Form 1 on storage at 40° C./75% RH and at 50% RH during the DVS cycle (FIG. 50-51). Storing the material at ambient temperature and humidity also saw partial conversion to Form 1.

### Preparation and Characterization of Form 9

Form 9 was scaled up using the following procedure with amorphous input material prepared according to the process described above. 3.2 mL of water was added to the lyophilized Compound A diacetate salt material to form a slurry. The slurry was then temperature cycled between ambient and 40° C. in 4 hour cycles with agitation for ca. 24 hours. The slurry was then filtered via centrifugation and the isolated solids collected into a scintillation vial and analyzed by XRPD. The saturated mother liquor was left at ambient to evaporate. A subsample was analyzed post evaporation by XRPD and the solids were dried in an oven at 40° C. for ca. 24 hours before being re-analyzed by XRPD. Solids were characterized using the same techniques as were used for Forms 3 and 8.

Thermal cycling of amorphous Compound A diacetate salt in water successfully produced Form 2 (consistent with the primary polymorph screen) which converted to Form 9 on drying at 40° C. Evaporation of the saturated mother liquor solution gave Form 2 which again dried successfully to Form 9 at 40° C. TG analysis identified a combined initial mass loss of 9.4% (0.3 equiv. water followed by 2.3 equiv. of water) associated with an endothermic event with an onset of 55.6° C. and a peak at 74.2° C., likely due to dehydration. A small exothermic event occurs prior to salt disproportionation with an onset of 146.7° C. and a peak at 156.1° C. A 31.7% weight loss (2.1 equiv. acetic acid) then occurs with a large endothermic event with an onset of 194.9° C. and a peak at 214.2° C. corresponding to salt disproportionation (FIG. 55). The DSC first heat identified a large endothermic event with an onset of 88.8° C. and a peak at 88.9° C. with a shoulder peak at 98.0° C. (FIG. 56). No significant thermal events were then present in the DSC cool or 2nd heat (FIG. 57-58).

The ¹H NMR spectrum is presented in FIG. 54. The spectrum was consistent with the diacetate structure with identified significant water content. PLM analysis identified large, highly birefringent rod-like morphology (FIG. 59). The FTIR spectrum was consistent with the expected diacetate structure, with the water peak hidden under the broad peak due to the O—H stretch ca. 3000 cm-1 (FIG. 60).

The DVS isotherm plot is presented in FIG. 61. The material undergoes a form change to Form 2 on the first sorption cycle at 80% RH with a 5.2 wt. % increase (1.4 equiv. water) and then convert back to Form 9 at 50% RH. Form 9 is then stable until dehydration below 10% RH where a 7.3 wt. % loss occurs (2.0 equiv. water). The kinetic plot shows the sample weight taking longer to stabilize on the 80-90% RH stage while the material converts from Form 9 to Form 2 (FIG. 62). XRPD analysis of the recovered solids found the sample to be a mixture of phases with peaks matching Form 9 and new peaks that are not consistent with other peaks seen. VHXRPD confirmed the Form 9 solids to convert to Form 2 at 90% RH however on drying the material at 0% RH the material becomes poorly crystalline and does not convert back to Form 9 when the humidity is increased to 80% RH.

The results of the one-week stability assessment are presented in Table 16. The Form 9 remained unchanged by XRPD post storage at 40° C./75% RH and under ambient temperature and humidity but was seen to convert to Form 1 when storage at 80° C. The material was dried by TG/DSC which identified a mass loss of 8.3% (2.1 equiv. of water) associated with a large, broad endothermic event with an onset of 60.7° C. and a peak at 81.6° C. (FIG. 63). XRPD analysis of the recovered solids found the material to be poorly crystalline with some small peaks corresponding to Form 1 and some new peaks.

Based on the results obtained, Form 9 appears to be a dihydrate that converts to the tetrahydrate Form 2 under high humidity (>80% RH). The material was seen to convert to Form 1 when stored at 80° C. for one week but does not appear to dehydrate to Form 1 on drying.

In summary, Form 9 was isolated from water and had good crystallinity by XRPD analysis. Similarly, PLM analysis found large, highly birefringent rod-like morphology. The material was found to be hydrated with TG/DSC showing a two-step mass loss of 1.3% (0.3 equiv. water) followed by 8.1% (2.3 equiv. water) and a melt at 194.9° C. DSC identified a sharp endothermic event corresponding to dehydration at 88.8° C. The Form 9 material was seen to convert to Form 2 at 80% RH in the DVS cycle and convert back to Form 9 at 50% RH (FIG. 61-62). This was confirmed by VH-XRPD. VH-XRPD analysis found on dehydration of Form 9 below 10% RH, the material becomes poorly crystalline and does not give Form 1. On increasing the humidity from 0% RH, Form 9 was not reformed even at 80% RH for one hour.

A summary of the additional characterization of forms 1, 3, 8, and 9 is provided in Table 16, below.

TABLE 16

| | Compound A Polymorphic Form | | | |
|---|---|---|---|---|
| Characterization | Form 1 | Form 3 | Form 8 | Form 9 |
| Crystallinity | Good | Average | Average | Good |
| TG/DSC | Anhydrous. | 11.7% mass loss (0.9 equiv. | 6.7% mass loss (0.7 equiv. | 2 step mass loss: 1.3% (0.3 |
| | Disproportionation/ | IPA of 3.0 equiv. water) Melt at | ethanol or 1.7 equiv. water) | equiv. water) followed by 6.1% |
| | sample melt | 192.2° C. | Melt at 196.0° C. | (2.3 equiv. water) Melt at |
| | at 193.1° C. | | | 194.9° C. |
| 1st Heat by DSC | Sample melt at 220.4° C. | Endothermic event at 120.9° C. | Two broad events at 32.5° C. | Sharp endothermic evant at |
| | | | and 78.8° C. | 88.8° C. |
| PLM | No clear morphology, Small, | No clear morphology, small, | No clear morphology, small, | Large, highly birefringent rod- |
| | poorly birefringent | highly birefringent particles | highly birefringent particles | like morphology |
| | particles present | visible | visible | |
| ¹H NMR | Consistent with supplied | 1.4 equiv. IPA present | 1.8 equiv. ethanol present | Significant water content |
| | structure | | | |
| FT-IR | Water peak at 3248.3 cm⁻¹ | Water peak at 3257.32 cm⁻¹ | No water peak identified | Water peak hidden under |
| | | | | broad peak |
| DVS | Slightly hygroscopic | Loss of 4 wt. % and changes to | De-solvation to pattern 1 at | Converts to pattern 2 at 80% |
| | with 1.8% mass | | | |
| | increase (0.5 equiv. | pattern 9 at 50% RM | 50% RH then slightly | RH then back to pattern 9 at |
| | water) at 90% RH. No change | | hygroscopic with a 1.6% mass | 50% RH. |
| | in form post-DVS | | increase at 90% RH. | |
| 1 Week Stability | No change in form by XRPD | Converts to pattern 1 at 40° C./ | Converts to pattern 1 at 40° C./ | Converts to pattern 1 at 80° C. |
| | under any of the relative | 75% RH and ambient | 75% RH and partial conversion | |
| | conditions | | at ambient | |
| De-solvation | N/A | Dries to pattern 1 above 150° C. | Loss of crystallinity on drying | Mixture obtained on drying at |
| | | | above 150° C. | 100° C. (some peaks matching |
| | | | | pattern 1 some new peaks) |

### Thermodynamic Solubility Assessment

The results of the thermodynamic solubility experiments are summarized in Table 17. All solids slurried in water were found to be amorphous by XRPD post-maturation. Form 1 was found to be the most soluble form at 49.5 mg/mL with Form 3 being the least soluble at 38.5 mg/mL. All solids slurried in a pH 7.4 phosphate buffer were found to be a new, poorly crystalline Form denoted Form 12 by XRPD analysis (FIG. 65). The material appears highly insoluble in the phosphate buffer with all Forms having a solubility of 0.3 mg/mL except from Form 2 which has a solubility of 0.2 mg/mL.

TABLE 17

| | | | Volume Added | | Solubility |
|---|---|---|---|---|---|
| No. | Input Material | Media | (μL) | XRPD Pattern | (mg/mL) |
| 1 | Form 1 | Water | 300 | Amorphous | 49.5 |
| 2 | Form 3 | Water | 400 | Amorphous | 38.5 |
| 3 | Form 8 | Water | 400 | Amorphous | 38.7 |
| 4 | Form 9 | Water | 300 | Amorphous | 44.0 |
| 5 | Form 1 | Phosphate Buffer | 500 | 12 | 0.3 |
| 6 | Form 3 | Phosphate Buffer | 500 | 12 | 0.2 |
| 7 | Form 8 | Phosphate Buffer | 500 | 12 | 0.3 |
| 8 | Form 9 | Phosphate Buffer | 500 | 12 | 0.3 |

Approximately 5 mg of Compound A diacetate salt Forms 1, 3, 8 and 9 were combined into 8×4 mL vials. The appropriate solvent was added to form a slurry and the samples were shaken at ambient or 60° C. for ca. 48 hours. The samples were then removed from shaking and centrifuge filtered. The isolated solids were analyzed by XRPD and were found to remain mixtures of forms. The solids were recovered from the XRPD plate and placed back on to shake as before for an additional 3 days before being re-analyzed by XRPD.

The results of the initial competitive slurry experiments are summarized in Table 18. All samples remained as mixtures of all input Compound A diacetate salt polymorphs after ca. 48 hours of agitation with the exception of the ethanol samples which had converted to a Form 1 and Form 8 mixture. After an additional 72 hours of agitation the ethanol at ambient sample had converted to Form 8 while all other samples remained unchanged.

TABLE 18

| | | Volume | | XRPD Pattern | |
|---|---|---|---|---|---|
| No. | Solvent | Added (μL) | Condition | (2 Days) | XRPD Pattern (5 Days) |
| 1 | DCM | 2000 | Ambient | All patterns | All patterns |
| 2 | Ethanol | 2000 | Ambient | 8 + 1 | 6 |
| 3 | Acetone | 2000 | Ambient | All patterns | All patterns |
| 4 | Ethyl Acetate | 2000 | Ambient | All patterns | All patterns |
| 5 | DCM | 1000 | 60° C. | All patterns | All patterns |
| 6 | Ethanol | 1000 | 60° C. | 8 + 1 | Insufficient Solids |
| 7 | Acetone | 1000 | 60° C. | All patterns | All patterns |
| 8 | Etyl Acetate | 1000 | 60° C. | All patterns | 1 + 8 |

### Table 18

Approximately 5 mg of Compound A diacetate salt Forms 1, 3, 8 and 9 were weighed out into 6×4 mL vials and ca. 10 mg of Forms 1 and 9 was weighed out into 3×2 mL vials. The appropriate solvent was then added to form a slurry and the samples were sealed with paraflilm and shaken at ambient, 40° C. or 60° C. for 9 days. The solids were analyzed by XRPD after 5 and 9 days of agitation.

The results of the additional competitive slurry experiments are summarized in Table 19. Majority of experiments remained as mixtures with the exception of 95% IPA, 5% water which returned Form 1 and 85% IPA, 150% water which returned Form 1 after 9 days of agitation.

TABLE 19

| | | | | Volume | XRPD Pattern | XRPD Pattern |
|---|---|---|---|---|---|---|
| No. | Input Material | Solvent | Condition | Added (μL) | ( [symbol] days) | ( [symbol] days) |
| 1 | 1, 3, [symbol] and 9 | Ethyl Acetate | Ambient | 2000 | All patterns | All patterns |
| 2 | 1, 3, [symbol] and 9 | Acetone | Ambient | 2000 | All patterns | All patterns |
| 3 | 1, 3, [symbol] and 9 | DCM | Ambient | 2000 | All patterns | All patterns |
| 4 | 1, 3, [symbol] and 9 | Ethyl Acetate | [symbol] | 1500 | All patterns | All patterns |
| 5 | 1, 3, [symbol] and 9 | Acetone | [symbol] | 1500 | All patterns | 3 and 1 |
| 6 | 1, 3, [symbol] and 9 | DCM | [symbol] | 1500 | All patterns | Primarily |
| | | | | | | amorphous |
| 7 | 1 and 9 | [symbol] | Ambient | 1000 | 3 and 1 | 3 and 1 |
| 8 | 1 and 9 | [symbol] | Ambient | 1000 | 1 | 1 |
| 9 | 1 and 9 | [symbol] | Ambient | 500 | Amorphous | 1 |

[symbol]

indicates data missing or illegible when filed

### Reactive Precipitation Experiments

Approximately 500 mg of the Compound A diacetate salt was weighed out and dissolved in 20 mL of water. The solution was added to a separating funnel and 20 mL of a saturated sodium bicarbonate solution was added. Immediate precipitation was noted. The slurry was washed with 3×40 mL of ethyl acetate before collecting the organic phase (containing the precipitate). This slurry was filtered using a Buchner filter and the isolated solids dried in a vacuum oven at 40° C. for ca. 2 hours before being analyzed by ¹H NMR.

Approximately 20 mg of Compound A free base was weighed out into 13×1.5 ml vials and an appropriate volume of solvent was added to form a slurry. 2.05 equivalents of acetic acid was then added to the slurries and the samples were sealed with parafilm and placed in an incubator shaker to temperature cycle between ambient and 40° C. in 4 hour cycles with agitation for ca. 48 hours. The samples were then removed from shaking, observations were recorded and the slurries centrifuge filtered. The isolated solids were analyzed by XRPD before the XRPD plate was dried in a 40° C. oven for ca. 72 hours and the samples re-analyzed to check for any changes in form. Where new forms were obtained, the solids were analyzed by TG/DSC, ¹H NMR, PLM and FT-IR where material amounts allowed.

The results of the reactive precipitation experiments are summarized in Table 20. One novel diffractogram was identified, denoted Form 13, from NMP and on drying Form 3 from THF. The majority of solvent systems gave Forms predictable from the primary polymorph screen. The XRPD diffractograms of Form 13 is illustrated in FIG. 66.

TABLE 20

| | | | | | XRPD | | | |
|---|---|---|---|---|---|---|---|---|
| | | Volume | Observation | | Pattern | Wet | Dry | |
| | | Added | Pre- | Post- | (Primary | XRPD | XRPD | |
| No. | Solvent | (μL) | Maturation | Maturation | Screen) | Pattern | Pattern | |
| 1 | 1,4-Dioxane | 500 | Slurry | Slurry | 3 | 3 | 3 | |
| 2 | 2-Propanol | 500 | Slurry | Slurry | 3 | 3 | 3 | 1 |
| 3 | Acetone | 500 | Slurry | Slurry | 3 | 3 + P | 11 | |
| 4 | Acetonitrile | 500 | Slurry | Slurry | 7 | 7 | 7 | |
| 5 | Ethanol | 500 | Slurry | Slurry | [symbol] | [symbol] | [symbol] | |
| 6 | Ethyl Acetate | 500 | Slurry | Slurry | [symbol] | [symbol] | [symbol] | |
| 7 | Methanol | 250 | Slurry | Slurry | 7 | 7 | 7 | |
| 8 | N,N-Dimethylacetamide | 500 | Slurry | Slurry | 4 | 4 | 4 | 7 |
| 9 | N-Methylpyrrolidone | 500 | Slurry | Slurry | 5 | 13 | 5 | 13 |

| | | | | | | | |
|---|---|---|---|---|---|---|---|
| 10 | Tetrahydrofuran | 500 | Slurry | Slurry | 2 | 3 | 13 |
| 11 | Toluene | 500 | Slurry | Slurry | 6 | 6 | 6 |
| 12 | Water | 350 | Slurry | Clear Solution | 2 | N/A | N/A |
| 13 | DCM | 500 | Slurry | Slurry | 3 | [symbol] | PC [symbol] |

[symbol]

indicates data missing or illegible when filed

### Example 11: Preparation of Formulations Containing Form 1 of Compound A

Batches of 30 mg and 120 mg strength capsules containing Form 1 of Compound A diacetate salt were prepared. The compositions of the 30 mg and 120 mg capsules are described in Table 21, below. The 30-mg strength were filled into size 2 HPMC capsules with a white body and flesh-colored cap. The 120-mg strength were filled into size 0 Swedish orange capsules.

TABLE 21

| | 30-mg Capsules | 120-mg Capsules |
|---|---|---|
| Ingredient | Quantity per Batch (grams) | |
| Form 1 of Compound A diacetate | 240.98 | 558.90 |
| salt | | |
| Silicified microcrystalline | 421.80 | 345.00 |
| cellulose | | |
| Lactose monohydrate | 210.99 | 172.50 |
| Colloidal silicon dioxide | 14.28 | 17.25 |
| Magnesium stearate | 4.46 | 5.18 |
| Number of capsules | 5950 | 3450 |

The capsules were prepared according to the process described in Scheme 2, below.

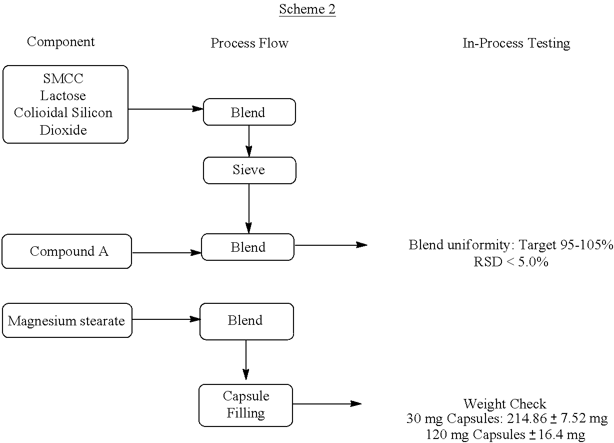

First, the required amounts of pre-sifted silicified microcrystalline cellulose (SMCC), lactose, and colloidal silicon dioxide were weighed into a PE bag and mixed manually for approximately 3 min. Then, the mixture was sieved through a 60-mesh screen. Then, Form 1 of Compound A diacetate salt was added to the mixture, and the mixture was blended manually for approximately 3 minutes. The mixture was then sampled for blend uniformity (95-100% of the label claim, with a % RSD of less than 5.0% was targeted). Next, magnesium stearate was added and the mixture was manually blended for approximately 2 minutes. The capsule shells were then manually filled with the blend. A weight check was then conducted (n=60 capsules). For the 30 mg capsules, a weight of 214±7.62 mg was targeted. For the 120 mg capsules, a weight of 422±16.4 mg was targeted. The capsules were then packaged in HDPE bottles with induction seals and child-resistant caps.

In addition, each reference, including all of the U.S. patents, U.S. patent application publications, U.S. patent applications, foreign patents, foreign patent applications and non-patent publications referred to in this specification are incorporated herein by reference, in their entirety, to the extent not inconsistent with the present description. Where a conflict exists between the instant application and a reference provided herein, the instant application shall dominate.

Although the foregoing invention has been described in some detail by way of illustration and example for purposes of clarity of understanding, one of skill in the art will appreciate that certain changes and modifications can be practiced within the scope of the appended claims.

It is to be understood that the invention is not limited to the described aspects illustrated herein above and the right is reserved to the illustrated aspects and all modifications coming within the scope of the claims.

1. A crystalline form of 5-((5-(4-carbamimidoylphenoxy)pentyl)oxy) picolinimidamide diacetate, or a solvate thereof.

2. The crystalline form of claim 1, wherein 5-((5-(4-carbamimidoylphenoxy)pentyl)oxy)picolinimidamide diacetate is non-solvated.

3. The crystalline form of claim 1, wherein the crystalline form is substantially anhydrous.

4. The crystalline form of claim 1, wherein the crystalline form is characterized as having an X-ray powder diffraction (XRPD) pattern comprising a peak at an angle 2-theta of about 17.86 degrees.

5. The crystalline form of claim 4, wherein the XRPD pattern further comprises a peak at an angle 2-theta of about 24.29 degrees.

6. The crystalline form of claim 4, wherein the XRPD pattern further comprises a peak at an angle 2-theta of about 24.21 degrees.

7. The crystalline form of claim 4, wherein the XRPD pattern further comprises a peak at an angle 2-theta of about 25.87 degrees.

8.-14. (canceled)

15. The crystalline form of claim 1, wherein 5-((5-(4-carbamimidoylphenoxy)pentyl)oxy)picolinimidamide diacetate is a solvate of isopropyl alcohol (IPA).

16. The crystalline form of claim 15, crystalline form is characterized as having an XRPD pattern comprising a peak at an angle 2-theta of about 25.35 degrees.

17. The crystalline form of claim 16, wherein the XRPD pattern further comprises a peak at an angle 2-theta of about 9.95 degrees.

18. The crystalline form of claim 16, wherein the XRPD pattern further comprises a peak at an angle 2-theta of about 17.88 degrees.

19.-44. (canceled)

45. The crystalline form of claim 1, wherein 5-((5-(4-carbamimidoylphenoxy)pentyl)oxy)picolinimidamide diacetate is a hydrate.

46. The crystalline form of claim 45, wherein the crystalline form is characterized as having an XRPD pattern comprising a peak at an angle 2-theta of about 10.14 degrees.

47. The crystalline form of claim 46, wherein the XRPD pattern further comprises a peak at an angle 2-theta of about 26.27 degrees.

48. The crystalline form of claim 46, wherein the XRPD pattern further comprises a peak at an angle 2-theta of about 24.77 degrees.

49.-54. (canceled)

55. The crystalline form of claim 1, wherein 5-((5-(4-carbamimidoylphenoxy)pentyl)oxy)picolinimidamide diacetate is a solvate of ethanol.

56. The crystalline form of claim 55, wherein the crystalline form is characterized as having an XRPD pattern comprising a peak at an angle 2-theta of about 5.53 degrees.

57. The crystalline form of claim 56, wherein the XRPD pattern further comprises a peak at an angle 2-theta of about 19.01 degrees.

58. The crystalline form of claim 56, wherein the XRPD pattern further comprises a peak at an angle 2-theta of about 24.63 degrees.

59.-65. (canceled)

66. The crystalline form of claim 45, wherein the crystalline form is characterized as having an XRPD pattern comprising a peak at an angle 2-theta of about 23.63 degrees.

67. The crystalline form of claim 66, wherein the XRPD pattern further comprises a peak at an angle 2-theta of about 26.06 degrees.

68. The crystalline form of claim 66, wherein the XRPD pattern further comprises a peak at an angle 2-theta of about 23.28 degrees.

69.-74. (canceled)

75. The crystalline form of claim 1, wherein the form is produced by a process comprising:
a. contacting 5-((5-(4-carbamimidoylphenoxy)pentyl)oxy)picolinimidamide diacetate with a solvent selected from the group consisting of 1,4-dioxane, 2-propanol, acetone, acetonitrile, ethanol, ethyl acetate, methanol, N,N-dimethylacetamide, N-methylpyrrolidone, tetrahydrofuran, toluene, water, DCM, or a mixture of any of the foregoing;
b. crystallizing 5-((5-(4-carbamimidoylphenoxy)pentyl)oxy)picolinimidamide diacetate, or a solvate thereof.

76. A pharmaceutical composition comprising the crystalline form of claim 1, and a pharmaceutically acceptable carrier or excipient.

77. The pharmaceutical composition of claim 76, wherein the pharmaceutical composition is a capsule comprising:
a. about 0.1% to about 60% w/w the crystalline form of claim 1;
b. about 10% to about 70% w/w silicified microcrystalline cellulose;
c. about 5% to about 30% w/w lactose monohydrate;
d. about 0.1% to about 5% w/w colloidal silicon dioxide; and
e. about 0.1% to about 5% w/w magnesium stearate.

78.-92. (canceled)

93. A method of treating cancer in an individual in need thereof comprising administering to the individual a therapeutically effective amount of the crystalline form of claim 1.

94. The method of claim 93, wherein the cancer is liver cancer.

95. The method of claim 94, wherein the liver cancer is hepatocellular carcinoma (HCC).

96. The method of claim 93, wherein the cancer is cancer of the lung, pancreas, colon, kidney, cholangiocarcinoma or breast.

97. The method of claim 93, where the cancer is cancer which initiates in the lung, colon, kidney, bile duct, pancreas or breast and then metastasizes to and grows in the liver.
